(19) United States
(12) Patent Application Publication
(10) Pub. No.: US 2024/0150757 A1
(43) Pub. Date: May 9, 2024

(54) ANTISENSE OLIGONUCLEOTIDES TARGETING FOXG1

(71) Applicant: EliGab Tx LLC, Manhasset, NY (US)

(72) Inventors: Scott REICH, Manhasset, NY (US); Hans-Peter VORNLOCHER, Kulmbach (DE); Anke GEICK, Kulmbach (DE); Brian BETTENCOURT, Manhasset, NY (US)

(21) Appl. No.: 18/336,603

(22) Filed: Jun. 16, 2023

Related U.S. Application Data
(63) Continuation of application No. PCT/US21/64082, filed on Dec. 17, 2021.
(60) Provisional application No. 63/127,907, filed on Dec. 18, 2020.

(51) Int. Cl. C12N 15/113; A61K 31/7125

(52) U.S. Cl. CPC C12N 15/113; A61K 31/7125; C12N 2310/11; C12N 2310/315; C12N 2310/321; C12N 2310/3231

(57) ABSTRACT

Provided herein are compositions and methods for treating and/or ameliorating FOXG1 syndrome or the symptoms associated therewith. The compositions and methods disclosed herein utilize antisense oligonucleotides that target FOXG1 in order to modulate FOXG1 by, for example, increasing the amount of FOXG1 (e.g. mRNA encoding a FOXG1 protein or FOXG1 protein) in a cell, thereby restoring FOXG1 function.

### CROSS-REFERENCE

This application claims the benefit of U.S. Provisional Patent Application No. 63/127,907, filed Dec. 18, 2020, which is incorporated herein by reference in its entirety.

### SEQUENCE LISTING

This application contains a Sequence Listing, which is incorporated by reference in its entirety. The accompanying Sequence Listing text file, named “2024-01-12 Revised_SL_ST26 062691-501C01US.xml” was created on Jan. 12, 2024 and is 2,123,592 bytes.

### BACKGROUND

FOXG1 syndrome is a rare neurodevelopmental disorder associated with heterozygous variants in the forkhead box G1 (FOXG1) gene and is characterized by impaired neurological development and/or altered brain physiology. Observed phenotypes of FOXG1 syndrome primarily include a particular pattern of structural alterations in the brain resulting from de novo mutations in the FOXG1 gene. Such structural alterations include a thin or underdeveloped corpus callosum that connects between the right and left hemispheres of the brain, reduced sulci and gyri formation on the surface of the brain, and/or a reduced amount of white matter. FOXG1 syndrome affects most aspects of development in children and the main clinical features observed in association with FOXG1 variants comprise impairment of postnatal growth, primary (congenital) or secondary (postnatal) microcephaly, severe intellectual disability with absent speech development, epilepsy, stereotypies and dyskinesia, abnormal sleep patterns, unexplained episodes of crying, gastroesophageal reflux, and recurrent aspiration.

### SUMMARY

Provided herein are compositions and methods for treating and/or ameliorating FOXG1 syndrome or the symptoms associated therewith. The compositions and methods disclosed herein utilize antisense oligonucleotides that target FOXG1 in order to modulate FOXG1 by, for example, increasing the amount of functional FOXG1 protein in a cell, thereby restoring or increasing FOXG1 function. The ability to restore or increase functional FOXG1 in cells provides for a foundation for the treatment of FOXG1 syndrome or alleviating symptoms associated therewith.

Accordingly, provided herein are antisense oligonucleotides, comprising a sequence complementary to a target nucleic acid sequence of a FOXG1 nucleic acid. In some embodiments, antisense oligonucleotide comprises a modification. In some embodiments, the modification comprises a modified inter-nucleoside linker, a modified nucleoside, or a combination thereof. In some embodiments, the antisense oligonucleotide comprises a modified inter-nucleoside linkage. In some embodiments, the modified inter-nucleoside linkage is a phosphorothioate inter-nucleoside linkage. In some embodiments, the antisense oligonucleotide comprises a phosphodiester inter-nucleoside linkage. In some embodiments, the antisense oligonucleotide comprises a modified nucleoside. In some embodiments, the modified nucleoside comprises a modified sugar. In some embodiments, the modified sugar is a bicyclic sugar. In some embodiments, the modified sugar comprises a 2′-O-methoxyethyl group. In some embodiments, the FOXG1 nucleic acid comprises a 5′ untranslated region (5′ UTR) and a 3′ untranslated region (3′ UTR), wherein, the target sequence is located at the 5′ UTR or the 3′ UTR of the FOXG1 nucleic acid.

In some embodiments, the target sequence is located at the 5′ UTR region of the FOXG1 nucleic acid. In some embodiments, the antisense oligonucleotide comprises a nucleobase sequence selected from the group consisting of SEQ ID NOs.: 1-84. In some embodiments, the target sequence is located at the 3′ UTR region of the FOXG1 nucleic acid. In some embodiments, the antisense oligonucleotide comprises a nucleobase sequence selected from the group consisting of SEQ ID NOs.: 85-384. In certain embodiments, the antisense oligonucleotide targeting the 3′ UTR comprises a nucleobase sequence complementary to a sequence within NM_005249.5_2000-2200_as region or NM_005249.5_2900-3000_as of the FOXG1 nucleic acid. In certain embodiments, the antisense oligonucleotide targeting the 3′ UTR comprises a nucleobase sequence selected from the group consisting SEQ ID NOs: 101, 103, 284, 2886, 287, 288, or 289. In some embodiments, the antisense oligonucleotides are included in an ASO composition comprising more than one ASO. In certain the embodiments, the ASO composition comprises 2, 3, 4, 5 or more ASOs. Such ASO compositions are suitable for use in the methods described herein.

In some embodiments, the antisense oligonucleotide is a single-stranded modified oligonucleotide. In some embodiments, the FOXG1 nucleic acid molecule is a ribonucleic acid (RNA). In some embodiments, the RNA molecule is a messenger RNA (mRNA) molecule. In some embodiments, the antisense oligonucleotide inhibits regulatory elements (e.g. miRNA suppression, suppression by nucleic acid-binding proteins, etc.) that reduce translation of the FOXG1 RNA. In some embodiments, the antisense oligonucleotide inhibits regulatory elements that reduce stability of the FOXG1 RNA. In some embodiments, the antisense oligonucleotide inhibits regulatory elements located within the 5′ UTR of the FOXG1 RNA. In some embodiments, the antisense oligonucleotide inhibits regulatory elements located within the 3′ UTR of the FOXG1 RNA. In some embodiments, the antisense oligonucleotide inhibits translation of an upstream open reading frame (uORF). In some embodiments, the antisense oligonucleotide sterically inhibits (1) miRNA binding and suppression of FOXG1 translation and/or (2) an RNA binding protein from binding to a regulatory sequence of the FOXG1 RNA and destabilizing the FOXG1 RNA. In some embodiments, the antisense oligonucleotide inhibits nuclease digestion of a 5′ region or 3′ region of the FOXG1 RNA. A pharmaceutical composition comprising the antisense oligonucleotide of an antisense oligonucleotide and a pharmaceutically acceptable carrier or diluent.

Also provided are methods of modulating expression of FOXG1 in a cell, comprising contacting the cell with a composition comprising an antisense oligonucleotide complementary to a target nucleic acid sequence of a FOXG1 nucleic acid.

In some embodiments, the cell is a located in a brain of an individual. In some embodiments, the individual is a human. In some embodiments, the individual comprises a mutated FOXG1 gene. In some embodiments, the individual has a FOXG1 disease or disorder. In some embodiments, the FOXG1 disease or disorder is FOXG1 syndrome. In some embodiments, the FOXG1 nucleic acid is a ribonucleic acid (RNA). In some embodiments, the RNA is a messenger RNA (mRNA).

In some embodiments, the antisense oligonucleotide inhibits regulatory elements that reduce translation or stability of the FOXG1 RNA, thereby increasing an amount of FOXG1 protein in a cell.

In some embodiments, the antisense oligonucleotide is a single-stranded modified oligonucleotide. In some embodiments, the antisense oligonucleotide comprises at least one modified inter-nucleoside linkage. In some embodiments, the modified inter-nucleoside linkage is a phosphorothioate inter-nucleoside linkage. In some embodiments, the antisense oligonucleotide comprises at least one phosphodiester inter-nucleoside linkage. In some embodiments, the antisense oligonucleotide comprises a modified nucleoside. In some embodiments, the modified nucleoside comprises a modified sugar. In some embodiments, the modified sugar is a bicyclic sugar. In some embodiments, the modified sugar comprises a 2′-O-methoxyethyl (MOE) group. In some embodiments, the antisense oligonucleotide comprises at least one phosphodiester inter-nucleoside linkage. In some embodiments, the target sequence is located at a 5′ UTR region or 3′ UTR region of the FOXG1 nucleic acid.

In some embodiments, the target sequence is located at the 5′ UTR region of the FOXG1 nucleic acid. In some embodiments, the antisense oligonucleotide comprises a nucleobase sequence selected from the group consisting of SEQ ID NOs.: 1-84. In some embodiments, the target sequence is located at the 3′ UTR region of the FOXG1 nucleic acid. In some embodiments, the antisense oligonucleotide comprises a nucleobase sequence selected from the group consisting of SEQ ID NOs.: 85-384. In some embodiments, modulating expression comprises increasing expression of a FOXG1 protein in the cell. In some embodiments, modulating expression comprises increasing stability or half-life of the FOXG1 nucleic acid in the cell. In some embodiments, modulating expression comprises increasing translation of a FOXG1 protein in the cell. In some embodiments, the antisense oligonucleotide is administered to the individual by intrathecal injection, intracerebroventricular injection, inhalation, parenteral injection or infusion, or orally.

Further provided are methods of treating or ameliorating a FOXG1 disease or disorder in an individual having, or at risk of having, the FOXG1 disease or disorder, comprising administering to the individual an antisense oligonucleotide, wherein the antisense oligonucleotide comprises a sequence complementary to a target sequence of the FOXG1 nucleic acid, thereby treating or ameliorating a FOXG1 disease in the individual. In some embodiments, the individual is a human. In some embodiments, the human is an unborn human. In some embodiments, the individual comprises a mutated FOXG1 gene. In some embodiments, the FOXG1 disease or disorder is FOXG1 syndrome. In some embodiments, the FOXG1 nucleic acid is a ribonucleic acid (RNA). In some embodiments, the RNA molecule is a messenger RNA (mRNA). In some embodiments, the target sequence is located at a 5′ UTR region or 3′ UTR region of the FOXG1 nucleic acid. In some embodiments, the target sequence is located at the 5′ UTR region of the FOXG1 nucleic acid. In some embodiments, the antisense oligonucleotide comprises a nucleobase sequence selected from the group consisting of SEQ ID NOs.: 1-84. In some embodiments, the target sequence is located at the 3′ UTR region of the FOXG1 nucleic acid. In some embodiments, the antisense oligonucleotide comprises a nucleobase sequence selected from the group consisting of SEQ ID NOs.: 85-384. In some embodiments, the antisense oligonucleotide modulates expression of the FOXG1 nucleic acid in the individual. In some embodiments, modulating expression comprises increasing stability or half-life of the FOXG1 nucleic acid in the individual. In some embodiments, modulating expression comprises increasing translation of a FOXG1 protein in the individual. In some embodiments, modulating expression comprises increasing translation of a FOXG1 protein in the individual. In some embodiments, modulating expression comprises increasing an amount of FOXG1 a cell of the individual. In some embodiments, the cell is located in the brain of the individual.

Also provided are antisense oligonucleotides comprising an antisense oligonucleotide sequence that hybridizes to a target nucleic acid sequence located within positions 2000-2100 or 2900-3000 of a FOXG1 nucleic acid (e.g., FOXG1 mRNA). In some embodiments, the antisense oligonucleotide comprises a modification. In some embodiments, the modification comprises a modified inter-nucleoside linker, a modified nucleoside, or a combination thereof. In some embodiments, the antisense oligonucleotide comprises a modified inter-nucleoside linkage. In some embodiments, the antisense oligonucleotide sequence comprises SEQ ID NO: 100, SEQ ID NO:103, SEQ ID NO: 284, SEQ ID NO: 286, SEQ ID NO: 287, SEQ ID NO: 288, or SEQ ID NO: 289. In some embodiments, the antisense oligonucleotide hybridizes to one or more nucleotides within or adjacent to a position on the FOXG1 nucleic acid targeted by SEQ ID NO: 100, SEQ ID NO:103, SEQ ID NO: 284, SEQ ID NO: 286, SEQ ID NO: 287, SEQ ID NO: 288, or SEQ ID NO: 289. In some embodiments, the antisense oligonucleotide hybridizes to one or more nucleotides within a position on the FOXG1 nucleic acid targeted by SEQ ID NO: 100, SEQ ID NO:103, SEQ ID NO: 284, SEQ ID NO: 286, SEQ ID NO: 287, SEQ ID NO: 288, or SEQ ID NO: 289. In some embodiments, the antisense oligonucleotide sequence comprises 80% sequence identity or greater to SEQ ID NO: 100, SEQ ID NO: 103, SEQ ID NO: 284, SEQ ID NO: 286, SEQ ID NO: 287, SEQ ID NO: 288, or SEQ ID NO: 289 In some embodiments, the antisense oligonucleotide sequence comprises 90% sequence identity or greater to SEQ ID NO: 100, SEQ ID NO: 103, SEQ ID NO: 284, SEQ ID NO: 286, SEQ ID NO: 287, SEQ ID NO: 288, or SEQ ID NO: 289. In some embodiments, the antisense oligonucleotide sequence comprises 10 or more contiguous nucleotides selected from a sequence within SEQ ID NO: 100, SEQ ID NO: 103, SEQ ID NO: 284, SEQ ID NO: 286, SEQ ID NO: 287, SEQ ID NO: 288, or SEQ ID NO: 289

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows a diagram of a FOXG1 transcript.

FIG. 2 shows FOXG1 mRNA expression of cells treated with ASOs targeting FOXG1 relative to mock transfection control

FIG. 3 shows FOXG1 mRNA expression modulation of 2′-O-methoxyethyl (MOE) chemistry antisense oligos in cells.

FIGS. 4A and 4B show FOXG1 mRNA expression modulation of selected 2′-O-methoxyethyl (MOE) chemistry antisense oligos in cells.

### DETAILED DESCRIPTION

Deletions or mutations in a single allele of the forkhead box G1 (FOXG1) gene cause FOXG1 syndrome. FOXG1 syndrome is a rare disease characterized by developmental delay, severe intellectual disability, epilepsy, absent language, and dyskinesis. Hallmarks of altered brain physiologies associated with FOXG1 syndrome include cortical atrophy and agenesis of the corpus callosum. The FOXG1 gene/protein is a member of the forkhead transcription factor family and is expressed specifically in neural progenitor cells of the forebrain. The FOXG1 gene is composed of one coding exon and notably, the location or type of FOXG1 mutation can be associated with or indicative of clinical severity.

The FOXG1 protein plays an important role in brain development, particularly in a region of the embryonic brain known as the telencephalon. The telencephalon ultimately develops into several critical structures, including the largest part of the brain (i.e. cerebrum), which controls most voluntary activity, language, sensory perception, learning, and memory. A shortage of functional FOXG1 protein, as observed in individuals having mutations or deletions in a single FOXG1 allele (i.e. heterozygous individuals), disrupts normal brain patterning and development.

Accordingly, disclosed herein are compositions and methods useful for increasing an amount of functional FOXG1 (e.g. FOXG1 protein or FOXG1 messenger ribonucleic acid (mRNA)) in a cell having a shortage of functional FOXG1. Such compositions and methods are useful in their application for treating individual having a FOXG1-related disease or disorder wherein the lack or shortage of functional FOXG1 protein can be remedied. In order to achieve an increase of FOXG1 expression in cells or in an individual, antisense oligonucleotides targeting FOXG1 are used.

### Antisense Oligonucleotides

Antisense oligonucleotides (ASOs) are small (˜18-30 nucleotides), synthetic, single-stranded nucleic acid polymers that can be employed to modulate gene expression by various mechanisms. Antisense oligonucleotides (ASOs) can be subdivided into two major categories: RNase H competent and steric block. For RNase H competent antisense oligonucleotides, the endogenous RNase H enzyme recognizes RNA-DNA heteroduplex substrates that are formed when antisense oligonucleotides bind to their cognate mRNA transcripts to catalyze the degradation of RNA. Steric block oligonucleotides are antisense oligonucleotides (ASOs) that are designed to bind to target transcripts with high affinity but do not induce target transcript degradation.

Steric block antisense oligonucleotides (ASOs) can be designed to inhibit translation inhibition, interfere with upstream open reading frames that negatively regulate translation in order to activate protein expression, inhibit RNA degradation, inhibit miRNA suppression, and influence polyadenylation signals to increase transcript stability. Accordingly, provided herein are steric block antisense oligonucleotides (ASOs) useful for modulating the expression and/or amount of functional FOXG1 (i.e. functional FOXG1) in a cell (e.g. mRNA encoding a functional FOXG1 protein or a FOXG1 protein). Specifically, the antisense oligonucleotides (ASOs) are useful for increasing the expression and/or amount of FOXG1 (i.e. functional FOXG1) in a cell (e.g. mRNA encoding a functional FOXG1 protein or a functional FOXG1 protein). The antisense oligonucleotides (ASOs) disclosed herein achieve this effect by targeting a FOXG1 nucleic acid encoding a functional FOXG1 protein and inhibiting translation inhibition, interfering with upstream open reading frames (uORFs), inhibiting RNA degradation, inhibiting miRNA suppression of expression, and/or increasing RNA stability to ultimately increase the number of RNA transcripts encoding FOXG1 and/or protein expression of a FOXG1 (i.e. functional FOXG1) protein.

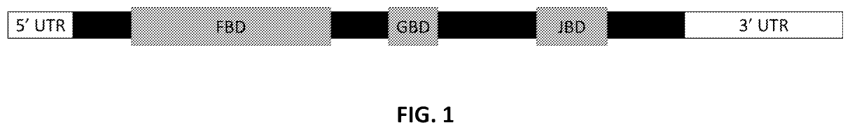

In order to achieve effective targeting of a FOXG1 RNA (e.g. messenger RNA), the antisense oligonucleotides disclosed herein (ASOs) comprise a sequence complementary to a sequence of the FOXG1 RNA, wherein the complementary sequence binds and/or hybridizes to a sequence of the FOXG1 RNA. Accordingly, disclosed herein are antisense oligonucleotides (ASOs) comprising a sequence complementary to a target nucleic acid sequence of a FOXG1 nucleic acid (e.g. a FOXG1 mRNA). Generally, mRNA transcripts comprise a 5′ untranslated region (5′ UTR) and a 3′ untranslated region (3′ UTR). The antisense oligonucleotides (ASOs) disclosed herein target the 5′ UTR or the 3′ UTR of a FOXG1 mRNA transcript. In order to achieve targeting of the 5′ UTR or 3′ UTR, the antisense oligonucleotide (ASOs) comprise a sequence complementary to a target sequence is located at the 5′ UTR or the 3′ UTR of the FOXG1 mRNA. In some embodiments, the target sequence is located at or within the 5′ UTR. In certain embodiments, the antisense oligonucleotide targeting the 5′ UTR comprises a nucleobase sequence selected from the group consisting of SEQ ID NO.: 1-84. In some embodiments, the target sequence is located at or within the 3′ UTR. In certain embodiments, the antisense oligonucleotide targeting the 3′ UTR comprises a nucleobase sequence selected from the group consisting of SEQ ID NO.: 85-384. In certain embodiments, the antisense oligonucleotide targeting the 3′ UTR comprises a nucleobase sequence complementary to a sequence within NM_005249.5_2000-2200_as region or NM_005249.5_2900-3000_as of the FOXG1 nucleic acid. In certain embodiments, the antisense oligonucleotide targeting the 3′ UTR comprises a nucleobase sequence selected from the group consisting SEQ ID NOs: 101, 103, 284, 2886, 287, 288, or 289. In some embodiments, the antisense oligonucleotides are included in an ASO composition comprising more than one ASO. In certain the embodiments, the ASO composition comprises 2, 3, 4, 5 or more ASOs. Such ASO compositions are suitable for use in the methods described herein. FIG. 1 shows a diagram of the FOXG1 mRNA transcript comprising 5′ and 3′ UTRs. TABLE 1 discloses sequences and antisense oligonucleotides (ASOs) having sequences complementary to the 5′ UTR of a FOXG1 mRNA. TABLE 2 discloses sequences and antisense oligonucleotides (ASOs) having sequences complementary to the 3′ UTR of a FOXG1 mRNA. In some embodiments, the antisense oligonucleotides (ASOs) disclosed herein, targeting the 5′ UTR or 3′ UTR, increase an amount of FOXG1 protein and/or mRNA transcripts in a cell and/or individual. In certain embodiments, targeting a FOXG1 nucleic acid encoding a functional FOXG1 protein inhibits translation inhibition, interferes with upstream open reading frames (uORFs), inhibits RNA degradation, and/or increases RNA stability to ultimately increase protein expression of a functional FOXG1 protein.

In order to improve the pharmacodynamic, pharmacokinetic, and biodistribution properties of antisense oligonucleotides (ASOs), the antisense oligonucleotides can be designed and engineered to comprise one or more chemical modifications (e.g. a modified inter-nucleoside linker, a modified nucleoside, or a combination thereof). Accordingly, in some embodiments, the antisense oligonucleotide is a modified oligonucleotide. In some embodiments, the antisense oligonucleotide comprises one or more modifications. In certain embodiments, the modification comprises a modified inter-nucleoside linker, a modified nucleoside, or a combination thereof

### Modified Inter-Nucleoside Linkers

Modification of the inter-nucleoside linker (i.e. backbone) can be utilized to increase pharmacodynamic, pharmacokinetic, and biodistribution properties. For example, inter-nucleoside linker modifications prevent or reduce degradation by cellular nucleases, thus increasing the pharmacokinetics and bioavailability of the antisense oligonucleotide. Generally, a modified inter-nucleoside linker includes any linker other than other than phosphodiester (PO) liners, that covalently couples two nucleosides together. In some embodiments, the modified inter-nucleoside linker increases the nuclease resistance of the antisense oligonucleotide compared to a phosphodiester linker. For naturally occurring antisense oligonucleotides, the inter-nucleoside linker includes phosphate groups creating a phosphodiester bond between adjacent nucleosides. Modified inter-nucleoside linkers are particularly useful in stabilizing antisense oligonucleotides for in vivo use and may serve to protect against nuclease cleavage.

In some embodiments, the antisense oligonucleotide comprises one or more inter-nucleoside linkers modified from the natural phosphodiester to a linker that is for example more resistant to nuclease attack. In some embodiments all of the inter-nucleoside linkers of the antisense oligonucleotide, or contiguous nucleotide sequence thereof, are modified. In some embodiments all of the inter-nucleoside linkers of the antisense oligonucleotide, or contiguous nucleotide sequence thereof, are nuclease resistant inter-nucleoside linkers. In some embodiments the inter-nucleoside linkage comprises sulphur (S), such as a phosphorothioate inter-nucleoside linkage.

Phosphorothioate inter-nucleoside linkers are particularly useful due to nuclease resistance and improved pharmacokinetics. In some embodiments, one or more of the inter-nucleoside linkers of the antisense oligonucleotide, or contiguous nucleotide sequence thereof, comprise a phosphorothioate inter-nucleoside linker. In some embodiments, all of the inter-nucleoside linkers of the antisense oligonucleotide, or contiguous nucleotide sequence thereof, comprise a phosphorothioate inter-nucleoside linker.

### Modified Nucleosides

Modifications to the ribose sugar or nucleobase can also be utilized to increase pharmacodynamic, pharmacokinetic, and biodistribution properties. Similar to modifications of the inter-nucleoside linker, nucleoside modifications prevent or reduce degradation by cellular nucleases, thus increasing the pharmacokinetics and bioavailability of the antisense oligonucleotide. Generally, a modified nucleoside includes the introduction of one or more modifications of the sugar moiety or the nucleobase moiety.

The antisense oligonucleotides, as described, can comprise one or more nucleosides comprising a modified sugar moiety, wherein the modified sugar moiety is a modification of the sugar moiety when compared to the ribose sugar moiety found in deoxyribose nucleic acid (DNA) and RNA. Numerous nucleosides with modification of the ribose sugar moiety can be utilized, primarily with the aim of improving certain properties of oligonucleotides, such as affinity and/or nuclease resistance. Such modifications include those where the ribose ring structure is modified. These modifications include replacement with a hexose ring (HNA), a bicyclic ring having a biradicle bridge between the C2 and C4 carbons on the ribose ring (e.g. locked nucleic acids (LNA)), or an unlinked ribose ring which typically lacks a bond between the C2 and C3 carbons (e.g. UNA). Other sugar modified nucleosides include, for example, bicyclohexose nucleic acids or tricyclic nucleic acids. Modified nucleosides also include nucleosides where the sugar moiety is replaced with a non-sugar moiety, for example in the case of peptide nucleic acids (PNA), or morpholino nucleic acids.

Sugar modifications also include modifications made by altering the substituent groups on the ribose ring to groups other than hydrogen, or the 2′-OH group naturally found in DNA and RNA nucleosides. Substituents may, for example be introduced at the 2′, 3′, 4′ or 5′ positions. Nucleosides with modified sugar moieties also include 2′ modified nucleosides, such as 2′ substituted nucleosides. Indeed, much focus has been spent on developing 2′ substituted nucleosides, and numerous 2′ substituted nucleosides have been found to have beneficial properties when incorporated into oligonucleotides, such as enhanced nucleoside resistance and enhanced affinity. A 2′ sugar modified nucleoside is a nucleoside that has a substituent other than H or —OH at the 2′ position (2′ substituted nucleoside) or comprises a 2′ linked biradicle, and includes 2′ substituted nucleosides and LNA (2′-4′ biradicle bridged) nucleosides. Examples of 2′ substituted modified nucleosides are 2′-O-alkyl-RNA, 2′-O-methyl-RNA, 2′-alkoxy-RNA, 2′-O-methoxyethyl-oligos (MOE), 2′-amino-DNA, 2′-Fluoro-RNA, and 2′-F-ANA nucleoside. In some embodiments, the antisense oligonucleotide comprises one or more modified sugars. In some embodiments, the antisense oligonucleotide comprises only modified sugars. In certain embodiments, the antisense oligo comprises greater than 10%, 25%, 50%, 75%, or 90% modified sugars. In some embodiments, the modified sugar is a bicyclic sugar. In some embodiments, the modified sugar comprises a 2′-O-methoxyethyl (MOE) group.

In some embodiments, the antisense oligonucleotide comprises both inter-nucleoside linker modifications and nucleoside modifications.

### Pharmaceutical Compositions

Further provided herein are pharmaceutical compositions comprising any of the disclosed antisense oligonucleotides and a pharmaceutically acceptable diluent, carrier, salt and/or adjuvant. A pharmaceutically acceptable diluent includes phosphate-buffered saline (PBS) and pharmaceutically acceptable salts include, but are not limited to, sodium and potassium salts. In some embodiments the pharmaceutically acceptable diluent is sterile phosphate buffered saline. In some embodiments the oligonucleotide is used in the pharmaceutically acceptable diluent at a concentration of 50-300 μM solution. In some embodiments, the oligonucleotide, as described, is administered at a dose of 10-1000 μg.

The antisense oligonucleotides or oligonucleotide conjugates of the disclosure may be mixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

### Methods of Use

The antisense oligonucleotides (ASOs) provided herein are useful for targeting a FOXG1 nucleic acid encoding a functional FOXG1 protein, wherein an antisense oligonucleotide inhibits translation inhibition, interferes with upstream open reading frames (uORFs), inhibits RNA degradation, and/or increases RNA stability to ultimately increase protein expression of a functional FOXG1 protein. According, the antisense oligonucleotides targeting are further useful in methods for increasing the expression and/or amount of functional FOXG1 in a cell (e.g. an amount of functional FOXG1 mRNA or protein). Accordingly, provided herein are methods of modulating expression of a FOXG1 in a cell, comprising contacting the cell with a composition comprising an antisense oligonucleotide complementary to a target nucleic acid sequence of a FOXG1 nucleic acid.

Further provided, are methods of treating or ameliorating a FOXG1 disease or disorder in an individual having, or at risk of having, the FOXG1 disease or disorder, comprising administering to the individual an antisense oligonucleotide, wherein the antisense oligonucleotide comprises a sequence complementary to a target sequence of the FOXG1 nucleic acid, thereby treating or ameliorating a FOXG1 disease in the individual.

Generally, cells of interest include neuronal cells and/or cells associated with the brain or brain development. In some embodiments, the cell is located in a brain of an individual. In some embodiments, the cell is a neural cell. In some embodiments, the individual is a human. In certain embodiments, the human is an unborn human.

The antisense oligonucleotides (ASOs) and methods are particularly useful for increasing the expression and/or amount of functional FOXG1 (e.g. an amount of functional FOXG1 mRNA or protein) in a cell and/or individual comprising a mutated or deleted FOXG1 allele. In some embodiments, the cell and/or individual comprises a mutated FOXG1 gene. In some embodiments the individual has been diagnosed with or at risk of a FOCG1 disease or disorder. In some embodiments the FOXG1 disease o disorder is FOXG1 syndrome.

In some embodiments, modulating expression comprises increasing expression of a FOXG1 protein in the cell. In some embodiments, modulating expression comprises increasing stability or half-life of the FOXG1 nucleic acid in the cell. In some embodiments, modulating expression comprises increasing translation of a FOXG1 protein in the cell.

In order to achieve effective targeting of a FOXG1 RNA (e.g. messenger RNA), the antisense oligonucleotides disclosed herein (ASOs) comprise a sequence complementary to a sequence of the FOXG1 RNA, wherein the complementary sequence binds and/or hybridizes to a sequence of the FOXG1 RNA. For example, mRNA transcripts comprise a 5′ untranslated region (5′ UTR) and a 3′ untranslated region (3′ UTR). The antisense oligonucleotides (ASOs) disclosed herein target the 5′ UTR or the 3′ UTR of a FOXG1 mRNA transcript. In order to achieve targeting of the 5′ UTR or 3′ UTR, the antisense oligonucleotide (ASOs) comprise a sequence complementary to a target sequence is located at the 5′ UTR or the 3′ UTR of the FOXG1 mRNA. In some embodiments, the target sequence is located at or within the 5′ UTR. In certain embodiments, the antisense oligonucleotide targeting the 5′ UTR comprises a nucleobase sequence selected from the group consisting of SEQ ID NO.: 1-84. In some embodiments, the target sequence is located at or within the 3′ UTR. In certain embodiments, the antisense oligonucleotide targeting the 3′ UTR comprises a nucleobase sequence selected from the group consisting of SEQ ID NO.: 85-384. In certain embodiments, the antisense oligonucleotide targeting the 3′ UTR comprises a nucleobase sequence complementary to a sequence within NM_005249.5_2000-2200_as region or NM_005249.5_2900-3000_as of the FOXG1 nucleic acid. In certain embodiments, the antisense oligonucleotide targeting the 3′ UTR comprises a nucleobase sequence complementary to a sequence within NM_005249.5_2000-2100_as region of the FOXG1 nucleic acid. In certain embodiments, the antisense oligonucleotide targeting the 3′ UTR comprises a nucleobase sequence selected from the group consisting SEQ ID NOs: 100, 103, 284, 2886, 287, 288, or 289. In some embodiments, the antisense oligonucleotides are included in an ASO composition comprising more than one ASO. In certain the embodiments, the ASO composition comprises 2, 3, 4, 5 or more ASOs.

Formulations of therapeutic and diagnostic agents can be prepared by mixing with physiologically acceptable carriers, excipients, or stabilizers in the form of, e.g., lyophilized powders, slurries, aqueous solutions, lotions, or suspensions (see, e.g., Hardman et al., Goodman and Gilman's The Pharmacological Basis of Therapeutics, McGraw-Hill, New York, N.Y., 2001; Gennaro, Remington: The Science and Practice of Pharmacy, Lippincott, Williams, and Wilkins, New York, N.Y., 2000; Avis, et al. (eds.), Pharmaceutical Dosage Forms: Parenteral Medications, Marcel Dekker, N Y, 1993; Lieberman, et al. (eds.), Pharmaceutical Dosage Forms: Tablets, Marcel Dekker, N Y, 1990; Lieberman, et al. (eds.) Pharmaceutical Dosage Forms: Disperse Systems, Inc., New York, N.Y., 2000).

Compositions comprising antisense oligonucleotides (ASOs), as disclosed herein, can be provided by doses at intervals of, e.g., one day, one week, or 1-7 times per week. A specific dose protocol is one involving the maximal dose or dose frequency that avoids significant undesirable side effects.

The disclosed antisense oligonucleotides or pharmaceutical compositions thereof can be administered topically (such as, to the skin, inhalation, ophthalmic or otic) or enterally (such as, orally or through the gastrointestinal tract) or parenterally (such as, intravenous, subcutaneous, intra-muscular, intracerebral, intracerebroventricular or intrathecal). In some embodiments the antisense oligonucleotide or pharmaceutical compositions thereof are administered by a parenteral route including intravenous, intraarterial, subcutaneous, intraperitoneal or intramuscular injection or infusion, intrathecal or intracranial, e.g. intracerebral or intraventricular, administration. In some embodiments the active oligonucleotide or oligonucleotide conjugate is administered intravenously.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The term “FOXG1,” as used herein, generally refers to the gene and gene products that encode a member of the fork-head transcription factor family. The encoded protein, which functions as a transcriptional repressor, is highly expressed in neural tissues during brain development. Mutations at this locus have been associated with Rett syndrome and a diverse spectrum of neurodevelopmental disorders defined as part of FOXG1 syndrome. Depending on the context of its use, “FOXG1” can refer to the FOXG1 gene, a FOXG1 deoxyribonucleic acid molecule (DNA), a FOXG1 ribonucleic acid molecule (RNA), or a FOXG1 protein. The mRNA sequence of FOXG1 is described in “NM_005249.5→NP_005240.3 forkhead box protein G1” or “accession number NM_005249.5” or the mRNA encoded by “NCBI GENE ID: 2290”. A functional FOXG1 protein describes the wild-type or unmutated FOXG1 gene, mRNA, and/or protein. Generally, “FOXG1” refers to a functional ‘FOXG1” gene or gene product, having normal function/activity within a cell. Deletions or mutations or variants of FOXG1 are indicative of non-functional FOXG1 variants having reduced, inhibited, or ablated FOXG1 function. As disclosed herein, the compositions and methods disclosed herein are primarily concerned with modulating or increasing or restoring an amount of FOXG1 (i.e. functional FOXG1) in a cell and/or individual.

The term “oligonucleotide,” as used herein, generally refers to a molecule comprising two or more covalently linked nucleosides. Such covalently bound nucleosides may also be referred to as nucleic acid molecules or oligomers. Oligonucleotides are commonly made in the laboratory by solid-phase chemical synthesis followed by purification. When referring to a sequence of the oligonucleotide, reference is made to the sequence or order of nucleobase moieties, or modifications thereof, of the covalently linked nucleotides or nucleosides. The oligonucleotide of the disclosure is man-made, and is chemically synthesized, and is typically purified or isolated. The oligonucleotide disclosed may comprise one or more modified nucleosides or nucleotides.

The term “antisense oligonucleotide,” as used herein, refers to oligonucleotides capable of modulating expression of a target gene by hybridizing to a target nucleic acid, in particular to a contiguous sequence on a target nucleic acid. Preferably, the antisense oligonucleotides of the present disclosure are single stranded. In some embodiments, the antisense oligonucleotide is single stranded.

The term “modified oligonucleotide” refers to an oligonucleotide comprising one or more sugar-modified nucleosides, modified nucleobases, and/or modified inter-nucleoside linkers.

The term “modified nucleoside” or “nucleoside modification,” as used herein, refers to nucleosides modified as compared to the equivalent DNA or RNA nucleoside by the introduction of one or more modifications of the sugar moiety or the (nucleo)base moiety. In some embodiments, the modified nucleoside comprise a modified sugar moiety. The term modified nucleoside may also be used herein interchangeably with the term “nucleoside analogue” or modified “units” or modified “monomers”.

The term “modified inter-nucleoside linkage” is refers to linkers other than phosphodiester (PO) linkers, that covalently couples two nucleosides together. Nucleotides with modified inter-nucleoside linkage are also termed “modified nucleotides”. In some embodiments, the modified inter-nucleoside linkage increases the nuclease resistance of the oligonucleotide compared to a phosphodiester linkage. For naturally occurring oligonucleotides, the inter-nucleoside linkage includes phosphate groups creating a phosphodiester bond between adjacent nucleosides. Modified inter-nucleoside linkers are particularly useful in stabilizing oligonucleotides for in vivo use and may serve to protect against nuclease cleavage at regions of DNA or RNA nucleosides.

The term “nucleobase” includes the purine (e.g. adenine and guanine) and pyrimidine (e.g. uracil, thymine and cytosine) moiety present in nucleosides and nucleotides which form hydrogen bonds in nucleic acid hybridization. The term nucleobase also encompasses modified nucleobases which may differ from naturally occurring nucleobases but are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants.

A nucleobase moiety can be modified by changing the purine or pyrimidine into a modified purine or pyrimidine, such as substituted purine or substituted pyrimidine, such as a nucleobased selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, and 5-methyl cytosine. In some embodiments, the cytosine nucleobases in a 5′cg3′ motif is 5-methyl cytosine.

The term “hybridizing” or “hybridizes” or “targets” or “binds” describes two nucleic acid strands (e.g. an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tm) defined as the temperature at which half of the oligonucleotides are duplexed with the target nucleic acid.

The oligonucleotide comprises a contiguous nucleotide region which is complementary to or hybridizes to a sub-sequence or region of the target nucleic acid molecule. The term “target sequence” as used herein refers to a sequence of nucleotides present in the target nucleic acid which comprises the nucleobase sequence which is complementary to the contiguous nucleotide region or sequence of the oligonucleotide of the disclosure. In some embodiments, the target sequence consists of a region on the target nucleic acid which is complementary to the contiguous nucleotide region or sequence of the oligonucleotide of the present disclosure. In some embodiments the target sequence is longer than the complementary sequence of a single oligonucleotide, and may, for example represent a preferred region of the target nucleic acid which may be targeted by several oligonucleotides of the present disclosure.

The oligonucleotide of the present disclosure comprises a contiguous nucleotide region which is complementary to a FOXG1 target nucleic acid, such as a target sequence of FOXG1.

The oligonucleotide comprises a contiguous nucleotide region of at least 10 nucleotides which is complementary to or hybridizes to a target sequence present in the target nucleic acid molecule. The contiguous nucleotide region (and therefore the target sequence) comprises of at least 10 contiguous nucleotides, such as 11, 12, 13, 14, 15, 16, 17, 18, 19, 20, 21, 22, 23, 24, 25, 30 contiguous nucleotides, such as from 15-30, such as from 18-23 contiguous nucleotides.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term “a therapeutically effective amount” of a compound of the present application refers to an amount of the compound of the present application that will elicit the biological or medical response of a subject, for example, reduction or inhibition of tumor cell proliferation, or ameliorate symptoms, alleviate conditions, slow or delay disease progression, or prevent a disease, etc. In one non-limiting embodiment, the term “a therapeutically effective amount” refers to the amount of a compound of the present application that, when administered to a subject, is effective to at least partially alleviate, inhibit, prevent and/or ameliorate a condition, or a disorder or a disease, or at least partially inhibit activity of a targeted enzyme or receptor.

As used in the specification and claims, the singular forms “a”, “an” and “the” include plural references unless the context clearly dictates otherwise. For example, the term “a sample” includes a plurality of samples, including mixtures thereof.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term “a therapeutically effective amount” of a compound of the present application refers to an amount of the compound of the present application that will elicit the biological or medical response of a subject, for example, reduction or inhibition of tumor cell proliferation, or ameliorate symptoms, alleviate conditions, slow or delay disease progression, or prevent a disease, etc. In one non-limiting embodiment, the term “a therapeutically effective amount” refers to the amount of a compound of the present application that, when administered to a subject, is effective to at least partially alleviate, inhibit, prevent and/or ameliorate a condition, or a disorder or a disease, or at least partially inhibit activity of a targeted enzyme or receptor.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

### EXEMPLARY EMBODIMENTS

Among the exemplary embodiments are:

Embodiment 1: An antisense oligonucleotide, comprising a sequence complementary to a target nucleic acid sequence of a FOXG1 nucleic acid.

Embodiment 2: The antisense oligonucleotide of embodiment 1, wherein antisense oligonucleotide comprises a modification.

Embodiment 3: The antisense oligonucleotide of embodiment 2, wherein the modification comprises a modified inter-nucleoside linker, a modified nucleoside, or a combination thereof.

Embodiment 4: The antisense oligonucleotide of embodiment 3, wherein the antisense oligonucleotide comprises a modified inter-nucleoside linkage.

Embodiment 5: The antisense oligonucleotide of embodiment 4, wherein the modified inter-nucleoside linkage is a phosphorothioate inter-nucleoside linkage.

Embodiment 6: The antisense oligonucleotide of any one of embodiments 3 to 5, wherein the antisense oligonucleotide comprises a phosphodiester inter-nucleoside linkage.

Embodiment 7: The antisense oligonucleotide of any one of embodiments 3 to 6, wherein the antisense oligonucleotide comprises a modified nucleoside.

Embodiment 8: The antisense oligonucleotide of embodiment 7, wherein the modified nucleoside comprises a modified sugar.

Embodiment 9: The antisense oligonucleotide of embodiment 8, wherein the modified sugar is a bicyclic sugar.

Embodiment 10: The antisense oligonucleotide of embodiment 8, wherein the modified sugar comprises a 2′-O-methoxyethyl (MOE) group.

Embodiment 11: The antisense oligonucleotide of any one of embodiments 1 to 10, wherein the FOXG1 nucleic acid comprises a 5′ untranslated region (5′ UTR) and a 3′ untranslated region (3′ UTR), and wherein the target sequence is located at the 5′ UTR or the 3′ UTR of the FOXG1 nucleic acid.

Embodiment 12: The antisense oligonucleotide of embodiment 11, wherein the target sequence is located at the 3′ UTR region of the FOXG1 nucleic acid.

Embodiment 13: The antisense oligonucleotide of embodiment 12, wherein the target sequence is located within a NM_005249.5_2000-2200_as region of the FOXG1 nucleic acid.

Embodiment 14: The antisense oligonucleotide of embodiment 13, wherein the antisense oligonucleotide comprises SEQ ID NO: 100 or SEQ ID NO:103.

Embodiment 15: The antisense oligonucleotide of embodiment 12, wherein the target sequence is located within a NM_005249.5_2900-3000_as region of the FOXG1 nucleic acid.

Embodiment 16: The antisense oligonucleotide of embodiment 13, wherein the antisense oligonucleotide comprises SEQ ID NO: 284, SEQ ID NO: 286, SEQ ID NO: 287, SEQ ID NO: 288, or SEQ ID NO: 289.

Embodiment 17: The antisense oligonucleotide of any one of embodiments 1 to 16, wherein the antisense oligonucleotide is a single-stranded modified oligonucleotide

Embodiment 18: The antisense oligonucleotide of any one of embodiments 1 to 17, wherein the FOXG1 nucleic acid molecule is a ribonucleic acid (RNA).

Embodiment 19: The antisense oligonucleotide of embodiment 18, wherein the RNA molecule is a messenger RNA (mRNA) molecule.

Embodiment 20: The antisense oligonucleotide of any one of embodiments 18 to 19, wherein the antisense oligonucleotide inhibits regulatory elements (e.g. miRNA suppression, suppression by nucleic acid-binding proteins, etc.) that reduce translation of the FOXG1 RNA.

Embodiment 21: The antisense oligonucleotide of any one of embodiments 18 to 19, wherein the antisense oligonucleotide inhibits regulatory elements that reduce stability of the FOXG1 RNA.

Embodiment 22: The antisense oligonucleotide of embodiment 21, wherein the antisense oligonucleotide inhibits regulatory elements (e.g. miRNA suppression, suppression by nucleic acid-binding proteins, etc.) located within the 3′ UTR of the FOXG1 RNA.

Embodiment 23: The antisense oligonucleotide of embodiment 21, wherein the antisense oligonucleotide sterically inhibits (1) miRNA binding and suppression of FOXG1 translation and/or (2) an RNA binding protein from binding to a regulatory sequence of the FOXG1 RNA and destabilizing the FOXG1 RNA.

Embodiment 24: The antisense oligonucleotide of embodiment 21, wherein the antisense oligonucleotide inhibits nuclease digestion of the FOXG1 RNA.

Embodiment 25: A pharmaceutical composition comprising the antisense oligonucleotide of any one of embodiments 1 to 24 and a pharmaceutically acceptable carrier or diluent.

Embodiment 26: A method of modulating expression of a FOXG1 in a cell, comprising contacting the cell with a composition comprising an antisense oligonucleotide complementary to a target nucleic acid sequence of a FOXG1 nucleic acid.

Embodiment 27: The method of embodiment 26, wherein the cell is a located in a brain of an individual.

Embodiment 28: The method of embodiment 27, wherein the individual is a human.

Embodiment 29: The method of embodiment 27, wherein the individual comprises a mutated FOXG1 gene.

Embodiment 30: The method of embodiment 27, wherein the individual has a FOXG1 disease or disorder.

Embodiment 31: The method of embodiment 30, wherein the FOXG1 disease or disorder is FOXG1 syndrome.

Embodiment 32: The method of any one of embodiments 26 to 31, wherein the FOXG1 nucleic acid is a ribonucleic acid (RNA).

Embodiment 33: The method of embodiment 32, wherein the RNA is a messenger RNA (mRNA).

Embodiment 34: The antisense oligonucleotide of any one of embodiments 32 to 33, wherein the antisense oligonucleotide inhibits regulatory elements (e.g. miRNA suppression, suppression by nucleic acid-binding proteins, nuclease digestion, etc.) that reduce translation or stability of the FOXG1 RNA, thereby increasing an amount of FOXG1 protein in a cell.

Embodiment 35: The method of any one of embodiments 26 to 34, wherein the antisense oligonucleotide is a single-stranded modified oligonucleotide.

Embodiment 36: The method of any one of embodiments 26 to 35, wherein the antisense oligonucleotide comprises at least one modified inter-nucleoside linkage.

Embodiment 37: The method of embodiment 36, wherein the modified inter-nucleoside linkage is a phosphorothioate inter-nucleoside linkage.

Embodiment 38: The method of any one of embodiments 26 to 37, wherein the antisense oligonucleotide comprises at least one phosphodiester inter-nucleoside linkage.

Embodiment 39: The method of any one of embodiments 26 to 38, wherein the antisense oligonucleotide comprises a modified nucleoside.

Embodiment 40: The method of embodiment 39, wherein the modified nucleoside comprises a modified sugar.

Embodiment 41: The method of embodiment 39, wherein the modified sugar is a bicyclic sugar.

Embodiment 42: The method of embodiment 39, wherein the modified sugar comprises a 2′-O-methoxyethyl group.

Embodiment 43: The method of any one of embodiments 26 to 42, wherein the antisense oligonucleotide comprises at least one phosphodiester inter-nucleoside linkage.

Embodiment 44: The method of any one of embodiments 27 to 43, wherein the target nucleic acid sequence is located at the 3′ UTR region of the FOXG1 nucleic acid.

Embodiment 45: The method of any one of embodiments 26 to 44, wherein the target sequence is located within a NM_005249.5_2000-2200_as region of the FOXG1 nucleic acid.

Embodiment 46: The method of embodiment 45, wherein the antisense oligonucleotide comprises SEQ ID NO: 100 or SEQ ID NO:103.

Embodiment 47: The method of any one of embodiments 26 to 44, wherein the target sequence is located within a NM_005249.5_2900-3000_as region of the FOXG1 nucleic acid.

Embodiment 48: The method of embodiment 47, wherein the antisense oligonucleotide comprises SEQ ID NO: 284, SEQ ID NO: 286, SEQ ID NO: 287, SEQ ID NO: 288, or SEQ ID NO: 289.

Embodiment 49: The method of any one of embodiments 26 to 48, wherein modulating expression comprises increasing expression of a FOXG1 protein in the cell.

Embodiment 50: The method of any one of embodiments 26 to 49, wherein modulating expression comprises increasing stability or half-life of the FOXG1 nucleic acid in the cell.

Embodiment 51: The method of any one of embodiments 26 to 50, wherein modulating expression comprises increasing translation of a FOXG1 protein in the cell.

Embodiment 52: The method of any one of embodiments 26 to 51, wherein the antisense oligonucleotide is administered to the individual by intrathecal injection, intracerebroventricular injection, inhalation, parenteral injection or infusion, or orally.

Embodiment 53: A method of treating or ameliorating a FOXG1 disease or disorder in an individual having, or at risk of having, the FOXG1 disease or disorder, comprising administering to the individual an antisense oligonucleotide, wherein the antisense oligonucleotide comprises a sequence complementary to a target sequence of the FOXG1 nucleic acid, thereby treating or ameliorating a FOXG1 disease in the individual.

Embodiment 54: The method of embodiment 53, wherein the individual is a human.

Embodiment 55: The method of embodiment 54, wherein the human is an unborn human.

Embodiment 56: The method of any one of embodiments 53 to 55, wherein the individual comprises a mutated FOXG1 gene.

Embodiment 57: The method of any one of embodiments 53 to 56, wherein the FOXG1 disease or disorder is FOXG1 syndrome.

Embodiment 58: The method of any one of embodiments 53 to 57, wherein the FOXG1 nucleic acid is a ribonucleic acid (RNA).

Embodiment 59: The method of embodiment 58, wherein the RNA molecule is a messenger RNA (mRNA).

Embodiment 60: The method of any one of embodiments 53 to 59, wherein the target sequence is located at a 3′ UTR region of the FOXG1 nucleic acid.

Embodiment 61: The method of any one of embodiments 53 to 60, wherein the target sequence is located within a NM_005249.5_2000-2200_as region of the FOXG1 nucleic acid.

Embodiment 62: The method of embodiment 61, wherein the antisense oligonucleotide comprises SEQ ID NO: 100, SEQ ID NO:103, or a combination thereof.

Embodiment 63: The method of any one of embodiments 53 to 60, wherein the target sequence is located within a NM_005249.5_2900-3000_as region of the FOXG1 nucleic acid.

Embodiment 64: The method of embodiment 63, wherein the antisense oligonucleotide comprises SEQ ID NO: 284, SEQ ID NO: 286, SEQ ID NO: 287, SEQ ID NO: 288, SEQ ID NO: 289, or any combination thereof.

Embodiment 65: The method of any one of embodiments 63 to 64, wherein the antisense oligonucleotide modulates expression of the FOXG1 nucleic acid in the individual.

Embodiment 66: The method of embodiment 65, wherein modulating expression comprises increasing stability or half-life of the FOXG1 nucleic acid in the individual.

Embodiment 67: The method of any one of embodiments 65 to 66, wherein modulating expression comprises increasing translation of a FOXG1 protein in the individual.

Embodiment 68: The method of any one of embodiments 65 to 66, wherein modulating expression comprises increasing translation of a FOXG1 protein in the individual.

Embodiment 69: The method of any one of embodiments 65 to 68, wherein modulating expression comprises increasing an amount of FOXG1 a cell of the individual.

Embodiment 70: The method of embodiment 69, wherein the cell is located in the brain of the individual.

Embodiment 71: The method of embodiment 70, wherein the cell is an astrocyte or a fibroblast.

Embodiment 72: The method of embodiment 27, wherein the cell is an astrocyte or a fibroblast

Embodiment 73: An antisense oligonucleotide comprising an antisense oligonucleotide sequence that hybridizes to a target nucleic acid sequence located within positions 2000-2100 or 2900-3000 of a FOXG1 nucleic acid (e.g., FOXG1 mRNA).

Embodiment 74: The antisense oligonucleotide of embodiment 73, wherein antisense oligonucleotide comprises a modification.

Embodiment 75: The antisense oligonucleotide of embodiment 74, wherein the modification comprises a modified inter-nucleoside linker, a modified nucleoside, or a combination thereof.

Embodiment 76 The antisense oligonucleotide of embodiment 75, wherein the antisense oligonucleotide comprises a modified inter-nucleoside linkage.

Embodiment 77: The antisense oligonucleotide of any one of embodiments 73 to 76, wherein the antisense oligonucleotide sequence comprises SEQ ID NO: 100, SEQ ID NO:103, SEQ ID NO: 284, SEQ ID NO: 286, SEQ ID NO: 287, SEQ ID NO: 288, or SEQ ID NO: 289.

Embodiment 78: The antisense oligonucleotide of any one of embodiments 73 to 76, wherein the antisense oligonucleotide hybridizes to one or more nucleotides within or adjacent to a position on the FOXG1 nucleic acid targeted by SEQ ID NO: 100, SEQ ID NO:103, SEQ ID NO: 284, SEQ ID NO: 286, SEQ ID NO: 287, SEQ ID NO: 288, or SEQ ID NO: 289.

Embodiment 79: The antisense oligonucleotide of any one of embodiments 73 to 76, wherein the antisense oligonucleotide hybridizes to one or more nucleotides within a position on the FOXG1 nucleic acid targeted by SEQ ID NO: 100, SEQ ID NO:103, SEQ ID NO: 284, SEQ ID NO: 286, SEQ ID NO: 287, SEQ ID NO: 288, or SEQ ID NO: 289.

Embodiment 80: The antisense oligonucleotide of any one of embodiments 73 to 79, wherein the antisense oligonucleotide sequence comprises 80% sequence identity or greater to SEQ ID NO: 100, SEQ ID NO: 103, SEQ ID NO: 284, SEQ ID NO: 286, SEQ ID NO: 287, SEQ ID NO: 288, or SEQ ID NO: 289

Embodiment 81: The antisense oligonucleotide of any one of embodiments 73 to 79, wherein the antisense oligonucleotide sequence comprises 90% sequence identity or greater to SEQ ID NO: 100, SEQ ID NO: 103, SEQ ID NO: 284, SEQ ID NO: 286, SEQ ID NO: 287, SEQ ID NO: 288, or SEQ ID NO: 289.

Embodiment 82: The antisense oligonucleotide of any one of embodiments 73 to 79, wherein the antisense oligonucleotide sequence comprises 10 or more contiguous nucleotides selected from a sequence within SEQ ID NO: 100, SEQ ID NO: 103, SEQ ID NO: 284, SEQ ID NO: 286, SEQ ID NO: 287, SEQ ID NO: 288, or SEQ ID NO: 289

### Examples

The following examples are included for illustrative purposes only and are not intended to limit the scope of the present disclosure.

### Example 1: Design and Selection of ASOs

Non-cleaving antisense oligonucleotides (“oligos”) against the human FOXG1 mRNA were chosen as follows. The full-length human FOXG1 mRNA (accession number NM_005249.5) was downloaded from the NCBI RefSeq database and served as template for all designs. All possible twenty-mer (“20 mer”) nucleotide subsequences that were reverse-complementary to the FOXG1 5′-UTR and 3′-UTR (NM_005249.5 coordinates 1-493 and 1964-3491, respectively) were assembled. Thermal and sequence characteristics were then used to initially subset the oligos as follows:
5′-UTR: GC content 15-70%; Tₘ 25-70° C.; Tₕₐᵢᵣₚᵢₙ<40° C.; T_{homodimer}<30° C.; no G homopolymers ≥4 bases long; no A, T, or C homopolymers ≥6 bases long
3′-UTR: GC content 20-60%; Tₘ 30-65° C.; Tₕₐᵢᵣₚᵢₙ<35° C.; T_{homodimer}<25° C.; no G homopolymers ≥4 bases long; no A, T, or C homopolymers ≥6 bases long

Different characteristics were used in the initial selection step (above) for 5′-UTR and 3′-UTR oligos due to the larger number of candidates for the 3′-UTR. In the above, Tₘ=Melting temperature of hybridization; Tₕₐᵢᵣₚᵢₙ=temperature of hairpin formation; T_{homodimer}=temperature of homodimer formation, as predicted by the Biopython software package (iittplibio.python.org).

These selected 20 mers were then further selected for specificity via sequence alignment to the complete human RefSeq unspliced transcriptome (downloaded Mar. 26, 2020). Alignment was conducted using the FASTA software suite (https://fasta.bioch.virginia.edu/fasta/fasta_list.html). Alignments were parsed using custom software, and the “off-target” score for each oligo was calculated as the lowest number of mismatches to any transcript other than FOXG1.

Next, the secondary structure of NM_005249.5 was predicted using the RNAstructure algorithm (https://rna.urmc.rochester.edu/RNAstructure.html). The oligo walk feature was used to predict the ΔG of target mRNA: oligo duplex formation with local structure invasion for each oligo. These predicted ΔG values were used in conjunction with off-target scores (above) to make the final selection of oligos as follows:
5′-UTR (84 oligos): ≥1 mismatch to all human off-target transcripts; no ΔG cutoff
3′-UTR (300 oligo): ≥2 mismatches to all human off-target transcripts; ΔG<−5.8° C.

The resulting set of 384 oligos, off-target scores, and ΔG values is listed in TABLE 1 and TABLE 2. In TABLE 1 and TABLE 2, exemplary chemical modifications are shown wherein “m” denotes 2′-O-Me bases, “d” denotes deoxyribo (DNA) bases, and “s” denotes phosphorothioate backbone.

TABLE 1
Antisense oligonucleotides targeting the 5′ UTR

| SEQ | | | Off- | | | |
|---|---|---|---|---|---|---|
| ID | NUCLEOBASE | | Target | ΔG | Exemplary Modified | SEQ ID |
| NO | SEQUENCE | Oligo Name | Score | Target | Sequence | NO |
| 1 | AGCGATCGA | NM_005249.5_ | 3 | −4.8 | mAsdGsmCsdGsmAsd | 385 |
| | GGCGGCTAT | 9-28_as | | | TsmCsdGsmAsdGsmG | |
| | AG | | | | sdCsmGsdGsmCsdTsm | |
| | | | | | AsdTsmAsdG | |
| 2 | CAGCGATCG | NM_005249.5_ | 3 | −16 | mCsdAsmGsdCsmGsd | 386 |
| | AGGCGGCTA | 10-29_as | | | AsmUsdCsmGsdAsmG | |
| | TA | | | | sdGsmCsdGsmGsdCs | |
| | | | | | mUsdAsmUsdA | |
| 3 | ACAGCGATC | NM_005249.5_ | 3 | −16.7 | mAsdCsmAsdGsmCsd | 387 |
| | GAGGCGGCT | 11-30_as | | | GsmAsdTsmCsdGsmA | |
| | AT | | | | sdGsmGsdCsmGsdGs | |
| | | | | | mCsdTsmAsdT | |
| 4 | GACAGCGAT | NM_005249.5_ | 3 | −14.1 | mGsdAsmCsdAsmGsd | 388 |
| | CGAGGCGGC | 12-31_as | | | CsmGsdAsmUsdCsmG | |
| | TA | | | | sdAsmGsdGsmCsdGs | |
| | | | | | mGsdCsmUsdA | |
| 5 | AGACAGCG | NM_005249.5_1 | 2 | −10.9 | mAsdGsmAsdCsmAsd | 389 |
| | ATCGAGGCG | 3-32_as | | | GsmCsdGsmAsdTsmC | |
| | GCT | | | | sdGsmAsdGsmGsdCs | |
| | | | | | mGsdGsmCsdT | |
| 6 | GCAGCAGTC | NM_005249.5_ | 1 | 16.4 | mGsdCsmAsdGsmCsd | 390 |
| | ACAGCAGCA | 106-125_as | | | AsmGsdTsmCsdAsmC | |
| | GC | | | | sdAsmGsdCsmAsdGs | |
| | | | | | mCsdAsmGsdC | |
| 7 | CGCAGCAGC | NM_005249.5_ | 2 | 0.4 | mCsdGsmCsdAsmGsd | 391 |
| | AGTCACAGC | 110-129_as | | | CsmAsdGsmCsdAsmG | |
| | AG | | | | sdTsmCsdAsmCsdAsm | |
| | | | | | GsdCsmAsdG | |
| 8 | TCGCAGCAG | NM_005249.5_ | 2 | −3.4 | mUsdCsmGsdCsmAsd | 392 |
| | CAGTCACAG | 111- | | | GsmCsdAsmGsdCsmA | |
| | CA | 130_as | | | sdGsmUsdCsmAsdCs | |
| | | | | | mAsdGsmCsdA | |
| 9 | CTCGCAGCA | NM_005249.5_ | 2 | −5.1 | mCsdTsmCsdGsmCsd | 393 |
| | GCAGTCACA | 112- | | | AsmGsdCsmAsdGsmC | |
| | GC | 131_as | | | sdAsmGsdTsmCsdAsm | |
| | | | | | CsdAsmGsdC | |
| 10 | TCTCGCAGC | NM_005249.5_ | 2 | −6.6 | mUsdCsmUsdCsmGsd | 394 |
| | AGCAGTCAC | 113- | | | CsmAsdGsmCsdAsmG | |
| | AG | 132_as | | | sdCsmAsdGsmUsdCs | |
| | | | | | mAsdCsmAsdG | |
| 11 | CTCTCGCAG | NM_005249.5_ | 2 | −10.9 | mCsdTsmCsdTsmCsd | 395 |
| | CAGCAGTCA | 114- | | | GsmCsdAsmGsdCsmA | |
| | CA | 133_as | | | sdGsmCsdAsmGsdTsm | |
| | | | | | CsdAsmCsdA | |
| 12 | CCTCTCGCA | NM_005249.5_ | 2 | −13.7 | mCsdCsmUsdCsmUsd | 396 |
| | GCAGCAGTC | 115- | | | CsmGsdCsmAsdGsmC | |
| | AC | 134_as | | | sdAsmGsdCsmAsdGs | |
| | | | | | mUsdCsmAsdC | |
| 13 | TCCTCTCGC | NM_005249.5_ | 2 | −16.7 | mUsdCsmCsdTsmCsd | 397 |
| | AGCAGCAGT | 116- | | | TsmCsdGsmCsdAsmG | |
| | CA | 135_as | | | sdCsmAsdGsmCsdAs | |
| | | | | | mGsdTsmCsdA | |
| 14 | CTCCTCTCG | NM_005249.5_ | 2 | −18.8 | mCsdTsmCsdCsmUsd | 398 |
| | CAGCAGCAG | 117- | | | CsmUsdCsmGsdCsmA | |
| | TC | 136_as | | | sdGsmCsdAsmGsdCs | |
| | | | | | mAsdGsmUsdC | |
| 15 | CCTCCTCTC | NM_005249.5_ | 2 | −22.6 | mCsdCsmUsdCsmCsd | 399 |
| | GCAGCAGCA | 118- | | | TsmCsdTsmCsdGsmCs | |
| | GT | 137_as | | | dAsmGsdCsmAsdGsm | |
| | | | | | CsdAsmGsdT | |
| 16 | TCCTCCTCT | NM_005249.5_ | 2 | −21.8 | mUsdCsmCsdTsmCsd | 400 |
| | CGCAGCAGC | 119- | | | CsmUsdCsmUsdCsmG | |
| | AG | 138_as | | | sdCsmAsdGsmCsdAs | |
| | | | | | mGsdCsmAsdG | |
| 17 | CTCCTCCTC | NM_005249.5_ | 2 | −22.7 | mCsdTsmCsdCsmUsd | 401 |
| | TCGCAGCAG | 120- | | | CsmCsdTsmCsdTsmCs | |
| | CA | 139_as | | | dGsmCsdAsmGsdCsm | |
| | | | | | AsdGsmCsdA | |
| 18 | TCCTCCTCC | NM_005249.5_ | 2 | −23.6 | mUsdCsmCsdTsmCsd | 402 |
| | TCTCGCAGC | 122- | | | CsmUsdCsmCsdTsmC | |
| | AG | 141_as | | | sdTsmCsdGsmCsdAsm | |
| | | | | | GsdCsmAsdG | |
| 19 | CTCCTCCTC | NM_005249.5_ | 1 | −20.1 | mCsdTsmCsdCsmUsd | 403 |
| | CTCTCGCAG | 123- | | | CsmCsdTsmCsdCsmU | |
| | CA | 142_as | | | sdCsmUsdCsmGsdCsm | |
| | | | | | AsdGsmCsdA | |
| 20 | TCCTCCTCC | NM_005249.5_ | 1 | −20.8 | mUsdCsmCsdTsmCsd | 404 |
| | TCCTCTCGC | 125- | | | CsmUsdCsmCsdTsmC | |
| | AG | 144_as | | | sdCsmUsdCsmUsdCsm | |
| | | | | | GsdCsmAsdG | |
| 21 | CTCCTCCTC | NM_005249.5_ | 1 | −17.3 | mCsdTsmCsdCsmUsd | 405 |
| | CTCCTCTCG | 126- | | | CsmCsdTsmCsdCsmU | |
| | CA | 145_as | | | sdCsmCsdTsmCsdTsm | |
| | | | | | CsdGsmCsdA | |
| 22 | GCTGCTTCC | NM_005249.5_ | 1 | −11.5 | mGsdCsmUsdGsmCsd | 406 |
| | TCCTCCTCC | 137- | | | TsmUsdCsmCsdTsmCs | |
| | TC | 156_as | | | dCsmUsdCsmCsdTsm | |
| | | | | | CsdCsmUsdC | |
| 23 | CGCTGCTTC | NM_005249.5_ | 1 | −7.9 | mCsdGsmCsdTsmGsd | 407 |
| | CTCCTCCTC | 138- | | | CsmUsdTsmCsdCsmU | |
| | CT | 157_as | | | sdCsmCsdTsmCsdCsm | |
| | | | | | UsdCsmCsdT | |
| 24 | TGTACTTCT | NM_005249.5_ | 2 | −14.3 | mUsdGsmUsdAsmCsd | 408 |
| | TGGTCTCCC | 179- | | | TsmUsdCsmUsdTsmG | |
| | CC | 198_as | | | sdGsmUsdCsmUsdCs | |
| | | | | | mCsdCsmCsdC | |
| 25 | CTGTACTTC | NM_005249.5_ | 2 | −17.5 | mCsdTsmGsdTsmAsd | 409 |
| | TTGGTCTCC | 180- | | | CsmUsdTsmCsdTsmU | |
| | CC | 199_as | | | sdGsmGsdTsmCsdTsm | |
| | | | | | CsdCsmCsdC | |
| 26 | ACTGTACTT | NM_005249.5_ | 2 | −15.7 | mAsdCsmUsdGsmUsd | 410 |
| | CTTGGTCTC | 181- | | | AsmCsdTsmUsdCsmU | |
| | CC | 200_as | | | sdTsmGsdGsmUsdCsm | |
| | | | | | UsdCsmCsdC | |
| 27 | AACTGTACT | NM_005249.5_ | 2 | −10.7 | mAsdAsmCsdTsmGsd | 411 |
| | TCTTGGTCT | 182- | | | TsmAsdCsmUsdTsmC | |
| | CC | 201_as | | | sdTsmUsdGsmGsdTsm | |
| | | | | | CsdTsmCsdC | |
| 28 | CAACTGTAC | NM_005249.5_ | 2 | −11.6 | mCsdAsmAsdCsmUsd | 412 |
| | TTCTTGGTC | 183- | | | GsmUsdAsmCsdTsmU | |
| | TC | 202_as | | | sdCsmUsdTsmGsdGsm | |
| | | | | | UsdCsmUsdC | |
| 29 | CCAACTGTA | NM_005249.5_ | 2 | −11.9 | mCsdCsmAsdAsmCsd | 413 |
| | CTTCTTGGT | 184- | | | TsmGsdTsmAsdCsmU | |
| | CT | 203_as | | | sdTsmCsdTsmUsdGsm | |
| | | | | | GsdTsmCsdT | |
| 30 | CCCAACTGT | NM_005249.5_ | 2 | −11 | mCsdCsmCsdAsmAsd | 414 |
| | ACTTCTTGG | 185- | | | CsmUsdGsmUsdAsmC | |
| | TC | 204_as | | | sdTsmUsdCsmUsdTsm | |
| | | | | | GsdGsmUsdC | |
| 31 | TCCCAACTG | NM_005249.5_ | 3 | −11 | mUsdCsmCsdCsmAsd | 415 |
| | TACTTCTTG | 186- | | | AsmCsdTsmGsdTsmA | |
| | GT | 205_as | | | sdCsmUsdTsmCsdTsm | |
| | | | | | UsdGsmGsdT | |
| 32 | CTCCCAACT | NM_005249.5_ | 2 | −13.8 | mCsdTsmCsdCsmCsd | 416 |
| | GTACTTCTT | 187- | | | AsmAsdCsmUsdGsmU | |
| | GG | 206_as | | | sdAsmCsdTsmUsdCsm | |
| | | | | | UsdTsmGsdG | |
| 33 | GCTCCCAAC | NM_005249.5_ | 2 | −15.3 | mGsdCsmUsdCsmCsd | 417 |
| | TGTACTTCT | 188- | | | CsmAsdAsmCsdTsmG | |
| | TG | 207_as | | | sdTsmAsdCsmUsdTsm | |
| | | | | | CsdTsmUsdG | |
| 34 | CGCTCCCAA | NM_005249.5_ | 2 | −14.8 | mCsdGsmCsdTsmCsd | 418 |
| | CTGTACTTC | 189- | | | CsmCsdAsmAsdCsmU | |
| | TT | 208_as | | | sdGsmUsdAsmCsdTsm | |
| | | | | | UsdCsmUsdT | |
| 35 | TCGCTCCCA | NM_005249.5_ | 2 | −12 | mUsdCsmGsdCsmUsd | 419 |
| | ACTGTACTT | 190- | | | CsmCsdCsmAsdAsmC | |
| | CT | 209_as | | | sdTsmGsdTsmAsdCsm | |
| | | | | | UsdTsmCsdT | |
| 36 | CTCGCTCCC | NM_005249.5_ | 2 | −11.5 | mCsdTsmCsdGsmCsd | 420 |
| | AACTGTACT | 191- | | | TsmCsdCsmCsdAsmA | |
| | TC | 210_as | | | sdCsmUsdGsmUsdAs | |
| | | | | | mCsdTsmUsdC | |
| 37 | CCTCGCTCC | NM_005249.5_ | 2 | −11.5 | mCsdCsmUsdCsmGsd | 421 |
| | CAACTGTAC | 192- | | | CsmUsdCsmCsdCsmA | |
| | TT | 211_as | | | sdAsmCsdTsmGsdTsm | |
| | | | | | AsdCsmUsdT | |
| 38 | CCCTCGCTC | NM_005249.5_ | 2 | −13.4 | mCsdCsmCsdTsmCsd | 422 |
| | CCAACTGTA | 193- | | | GsmCsdTsmCsdCsmC | |
| | CT | 212_as | | | sdAsmAsdCsmUsdGs | |
| | | | | | mUsdAsmCsdT | |
| 39 | TCCCTCGCT | NM_005249.5_ | 2 | −13.2 | mUsdCsmCsdCsmUsd | 423 |
| | CCCAACTGT | 194- | | | CsmGsdCsmUsdCsmC | |
| | AC | 213_as | | | sdCsmAsdAsmCsdTsm | |
| | | | | | GsdTsmAsdC | |
| 40 | CTCCCTCGC | NM_005249.5_ | 1 | −15.5 | mCsdTsmCsdCsmCsd | 424 |
| | TCCCAACTG | 195- | | | TsmCsdGsmCsdTsmCs | |
| | TA | 214_as | | | dCsmCsdAsmAsdCsm | |
| | | | | | UsdGsmUsdA | |
| 41 | GCTCCCTCG | NM_005249.5_ | 2 | −20.2 | mGsdCsmUsdCsmCsd | 425 |
| | CTCCCAACT | 196- | | | CsmUsdCsmGsdCsmU | |
| | GT | 215_as | | | sdCsmCsdCsmAsdAsm | |
| | | | | | CsdTsmGsdT | |
| 42 | AGCTCCCTC | NM_005249.5_ | 3 | −18.5 | mAsdGsmCsdTsmCsd | 426 |
| | GCTCCCAAC | 197- | | | CsmCsdTsmCsdGsmC | |
| | TG | 216_as | | | sdTsmCsdCsmCsdAsm | |
| | | | | | AsdCsmUsdG | |
| 43 | AAGCTCCCT | NM_005249.5_ | 2 | −16.1 | mAsdAsmGsdCsmUsd | 427 |
| | CGCTCCCAA | 198- | | | CsmCsdCsmUsdCsmG | |
| | CT | 217_as | | | sdCsmUsdCsmCsdCsm | |
| | | | | | AsdAsmCsdT | |
| 44 | GAAGCTCCC | NM_005249.5_ | 2 | −9.4 | mGsdAsmAsdGsmCsd | 428 |
| | TCGCTCCCA | 199- | | | TsmCsdCsmCsdTsmCs | |
| | AC | 218_as | | | dGsmCsdTsmCsdCsm | |
| | | | | | CsdAsmAsdC | |
| 45 | TGAAGCTCC | NM_005249.5_ | 2 | −11.1 | mUsdGsmAsdAsmGsd | 429 |
| | CTCGCTCCC | 200- | | | CsmUsdCsmCsdCsmU | |
| | AA | 219_as | | | sdCsmGsdCsmUsdCsm | |
| | | | | | CsdCsmAsdA | |
| 46 | GTGAAGCTC | NM_005249.5_ | 2 | −9.7 | mGsdTsmGsdAsmAsd | 430 |
| | CCTCGCTCC | 201- | | | GsmCsdTsmCsdCsmC | |
| | CA | 220_as | | | sdTsmCsdGsmCsdTsm | |
| | | | | | CsdCsmCsdA | |
| 47 | AAGAAACA | NM_005249.5_ | 3 | −5.7 | mAsdAsmGsdAsmAsd | 431 |
| | ACCACCGCC | 224- | | | AsmCsdAsmAsdCsmC | |
| | CCG | 243_as | | | sdAsmCsdCsmGsdCsm | |
| | | | | | CsdCsmCsdG | |
| 48 | AAAGAAAC | NM_005249.5_ | 2 | −5.7 | mAsdAsmAsdGsmAsd | 432 |
| | AACCACCGC | 225- | | | AsmAsdCsmAsdAsmC | |
| | CCC | 244_as | | | sdCsmAsdCsmCsdGsm | |
| | | | | | CsdCsmCsdC | |
| 49 | AAAAGAAA | NM_005249.5_ | 2 | −3 | mAsdAsmAsdAsmGsd | 433 |
| | CAACCACCG | 226- | | | AsmAsdAsmCsdAsmA | |
| | CCC | 245_as | | | sdCsmCsdAsmCsdCsm | |
| | | | | | GsdCsmCsdC | |
| 50 | AAAAAGAA | NM_005249.5_ | 2 | 0.1 | mAsdAsmAsdAsmAsd | 434 |
| | ACAACCACC | 227- | | | GsmAsdAsmAsdCsmA | |
| | GCC | 246_as | | | sdAsmCsdCsmAsdCsm | |
| | | | | | CsdGsmCsdC | |
| 51 | CCCCTCAGG | NM_005249.5_ | 2 | −4 | mCsdCsmCsdCsmUsd | 435 |
| | AATTAGAAA | 280- | | | CsmAsdGsmGsdAsmA | |
| | AA | 299_as | | | sdTsmUsdAsmGsdAs | |
| | | | | | mAsdAsmAsdA | |
| 52 | ACCCCTCAG | NM_005249.5_ | 2 | −3.9 | mAsdCsmCsdCsmCsd | 436 |
| | GAATTAGAA | 281- | | | TsmCsdAsmGsdGsmA | |
| | AA | 300_as | | | sdAsmUsdTsmAsdGs | |
| | | | | | mAsdAsmAsdA | |
| 53 | CACCCCTCA | NM_005249.5_ | 2 | −1.2 | mCsdAsmCsdCsmCsd | 437 |
| | GGAATTAGA | 282- | | | CsmUsdCsmAsdGsmG | |
| | AA | 301_as | | | sdAsmAsdTsmUsdAs | |
| | | | | | mGsdAsmAsdA | |
| 54 | CCACCCCTC | NM_005249.5_ | 2 | −0.8 | mCsdCsmAsdCsmCsd | 438 |
| | AGGAATTAG | 283- | | | CsmCsdTsmCsdAsmG | |
| | AA | 302_as | | | sdGsmAsdAsmUsdTs | |
| | | | | | mAsdGsmAsdA | |
| 55 | ACCACCCCT | NM_005249.5_ | 2 | −3.6 | mAsdCsmCsdAsmCsd | 439 |
| | CAGGAATTA | 284- | | | CsmCsdCsmUsdCsmA | |
| | GA | 303_as | | | sdGsmGsdAsmAsdTs | |
| | | | | | mUsdAsmGsdA | |
| 56 | AACCACCCC | NM_005249.5_ | 2 | −2.3 | mAsdAsmCsdCsmAsd | 440 |
| | TCAGGAATT | 285- | | | CsmCsdCsmCsdTsmCs | |
| | AG | 304_as | | | dAsmGsdGsmAsdAsm | |
| | | | | | UsdTsmAsdG | |
| 57 | CAACCACCC | NM_005249.5_ | 2 | 0.2 | mCsdAsmAsdCsmCsd | 441 |
| | CTCAGGAAT | 286- | | | AsmCsdCsmCsdCsmU | |
| | TA | 305_as | | | sdCsmAsdGsmGsdAs | |
| | | | | | mAsdTsmUsdA | |
| 58 | GCAACCACC | NM_005249.5_ | 3 | 0.8 | mGsdCsmAsdAsmCsd | 442 |
| | CCTCAGGAA | 287- | | | CsmAsdCsmCsdCsmC | |
| | TT | 306_as | | | sdTsmCsdAsmGsdGsm | |
| | | | | | AsdAsmUsdT | |
| 59 | AGCAACCAC | NM_005249.5_ | 2 | 1.8 | mAsdGsmCsdAsmAsd | 443 |
| | CCCTCAGGA | 288- | | | CsmCsdAsmCsdCsmC | |
| | AT | 307_as | | | sdCsmUsdCsmAsdGs | |
| | | | | | mGsdAsmAsdT | |
| 60 | CAGCAACCA | NM_005249.5_ | 2 | −7.1 | mCsdAsmGsdCsmAsd | 444 |
| | CCCCTCAGG | 289- | | | AsmCsdCsmAsdCsmC | |
| | AA | 308_as | | | sdCsmCsdTsmCsdAsm | |
| | | | | | GsdGsmAsdA | |
| 61 | GCAGCAACC | NM_005249.5_ | 1 | −9.6 | mGsdCsmAsdGsmCsd | 445 |
| | ACCCCTCAG | 290- | | | AsmAsdCsmCsdAsmC | |
| | GA | 309_as | | | sdCsmCsdCsmUsdCsm | |
| | | | | | AsdGsmGsdA | |
| 62 | AAGCAGCA | NM_005249.5_ | 1 | −7.6 | mAsdAsmGsdCsmAsd | 446 |
| | ACCACCCCT | 292- | | | GsmCsdAsmAsdCsmC | |
| | CAG | 311_as | | | sdAsmCsdCsmCsdCsm | |
| | | | | | UsdCsmAsdG | |
| 63 | AAAGCAGC | NM_005249.5_ | 2 | 2.4 | mAsdAsmAsdGsmCsd | 447 |
| | AACCACCCC | 293- | | | AsmGsdCsmAsdAsmC | |
| | TCA | 312_as | | | sdCsmAsdCsmCsdCsm | |
| | | | | | CsdTsmCsdA | |
| 64 | AAAAGCAG | NM_005249.5_ | 2 | 2.6 | mAsdAsmAsdAsmGsd | 448 |
| | CAACCACCC | 294- | | | CsmAsdGsmCsdAsmA | |
| | CTC | 313_as | | | sdCsmCsdAsmCsdCsm | |
| | | | | | CsdCsmUsdC | |
| 65 | CAAAAGCA | NM_005249.5_ | 2 | −1 | mCsdAsmAsdAsmAsd | 449 |
| | GCAACCACC | 295- | | | GsmCsdAsmGsdCsmA | |
| | CCT | 314_as | | | sdAsmCsdCsmAsdCsm | |
| | | | | | CsdCsmCsdT | |
| 66 | GCAAAAGC | NM_005249.5_ | 2 | −1.4 | mGsdCsmAsdAsmAsd | 450 |
| | AGCAACCAC | 296- | | | AsmGsdCsmAsdGsmC | |
| | CCC | 315_as | | | sdAsmAsdCsmCsdAs | |
| | | | | | mCsdCsmCsdC | |
| 67 | AGCAAAAG | NM_005249.5_ | 2 | 1 | mAsdGsmCsdAsmAsd | 451 |
| | CAGCAACCA | 297- | | | AsmAsdGsmCsdAsmG | |
| | CCC | 316_as | | | sdCsmAsdAsmCsdCsm | |
| | | | | | AsdCsmCsdC | |
| 68 | TAGCAAAAG | NM_005249.5_ | 1 | 0 | mUsdAsmGsdCsmAsd | 452 |
| | CAGCAACCA | 298- | | | AsmAsdAsmGsdCsmA | |
| | CC | 317_as | | | sdGsmCsdAsmAsdCs | |
| | | | | | mCsdAsmCsdC | |
| 69 | GTAGCAAAA | NM_005249.5_ | 2 | −2.6 | mGsdTsmAsdGsmCsd | 453 |
| | GCAGCAACC | 299- | | | AsmAsdAsmAsdGsmC | |
| | AC | 318_as | | | sdAsmGsdCsmAsdAs | |
| | | | | | mCsdCsmAsdC | |
| 70 | TGTAGCAAA | NM_005249.5_ | 1 | −5.3 | mUsdGsmUsdAsmGsd | 454 |
| | AGCAGCAAC | 300- | | | CsmAsdAsmAsdAsmG | |
| | CA | 319_as | | | sdCsmAsdGsmCsdAs | |
| | | | | | mAsdCsmCsdA | |
| 71 | ATGTAGCAA | NM_005249.5_ | 2 | −6.1 | mAsdTsmGsdTsmAsd | 455 |
| | AAGCAGCA | 301- | | | GsmCsdAsmAsdAsmA | |
| | ACC | 320_as | | | sdGsmCsdAsmGsdCs | |
| | | | | | mAsdAsmCsdC | |
| 72 | CATGTAGCA | NM_005249.5_ | 2 | −3.5 | mCsdAsmUsdGsmUsd | 456 |
| | AAAGCAGC | 302- | | | AsmGsdCsmAsdAsmA | |
| | AAC | 321_as | | | sdAsmGsdCsmAsdGs | |
| | | | | | mCsdAsmAsdC | |
| 73 | TCATGTAGC | NM_005249.5_ | 2 | −5.3 | mUsdCsmAsdTsmGsd | 457 |
| | AAAAGCAG | 303- | | | TsmAsdGsmCsdAsmA | |
| | CAA | 322_as | | | sdAsmAsdGsmCsdAs | |
| | | | | | mGsdCsmAsdA | |
| 74 | GTCATGTAG | NM_005249.5_ | 2 | −5.7 | mGsdTsmCsdAsmUsd | 458 |
| | CAAAAGCA | 304- | | | GsmUsdAsmGsdCsmA | |
| | GCA | 323_as | | | sdAsmAsdAsmGsdCs | |
| | | | | | mAsdGsmCsdA | |
| 75 | AGTCATGTA | NM_005249.5_ | 2 | −8.1 | mAsdGsmUsdCsmAsd | 459 |
| | GCAAAAGC | 305- | | | TsmGsdTsmAsdGsmC | |
| | AGC | 324_as | | | sdAsmAsdAsmAsdGs | |
| | | | | | mCsdAsmGsdC | |
| 76 | AAGTCATGT | NM_005249.5_ | 2 | −5.5 | mAsdAsmGsdTsmCsd | 460 |
| | AGCAAAAG | 306- | | | AsmUsdGsmUsdAsmG | |
| | CAG | 325_as | | | sdCsmAsdAsmAsdAs | |
| | | | | | mGsdCsmAsdG | |
| 77 | CAAGTCATG | NM_005249.5_ | 1 | −5.7 | mCsdAsmAsdGsmUsd | 461 |
| | TAGCAAAAG | 307- | | | CsmAsdTsmGsdTsmA | |
| | CA | 326_as | | | sdGsmCsdAsmAsdAs | |
| | | | | | mAsdGsmCsdA | |
| 78 | GCAAGTCAT | NM_005249.5_ | 2 | −8.1 | mGsdCsmAsdAsmGsd | 462 |
| | GTAGCAAAA | 308- | | | TsmCsdAsmUsdGsmU | |
| | GC | 327_as | | | sdAsmGsdCsmAsdAs | |
| | | | | | mAsdAsmGsdC | |
| 79 | GGCAAGTCA | NM_005249.5_ | 2 | −10.4 | mGsdGsmCsdAsmAsd | 463 |
| | TGTAGCAAA | 309- | | | GsmUsdCsmAsdTsmG | |
| | AG | 328_as | | | sdTsmAsdGsmCsdAsm | |
| | | | | | AsdAsmAsdG | |
| 80 | TGGCAAGTC | NM_005249.5_ | 2 | −9.2 | mUsdGsmGsdCsmAsd | 464 |
| | ATGTAGCAA | 310- | | | AsmGsdTsmCsdAsmU | |
| | AA | 329_as | | | sdGsmUsdAsmGsdCs | |
| | | | | | mAsdAsmAsdA | |
| 81 | CTGGCAAGT | NM_005249.5_ | 2 | −11.1 | mCsdTsmGsdGsmCsd | 465 |
| | CATGTAGCA | 311- | | | AsmAsdGsmUsdCsmA | |
| | AA | 330_as | | | sdTsmGsdTsmAsdGsm | |
| | | | | | CsdAsmAsdA | |
| 82 | GCTGGCAAG | NM_005249.5_ | 2 | −12.5 | mGsdCsmUsdGsmGsd | 466 |
| | TCATGTAGC | 312- | | | CsmAsdAsmGsdTsmC | |
| | AA | 331_as | | | sdAsmUsdGsmUsdAs | |
| | | | | | mGsdCsmAsdA | |
| 83 | CGCTGGCAA | NM_005249.5_ | 2 | −10.6 | mCsdGsmCsdTsmGsd | 467 |
| | GTCATGTAG | 313- | | | GsmCsdAsmAsdGsmU | |
| | CA | 332_as | | | sdCsmAsdTsmGsdTsm | |
| | | | | | AsdGsmCsdA | |
| 84 | GCGCTGGCA | NM_005249.5_ | 3 | -14.6 | mGsdCsmGsdCsmUsd | 468 |
| | AGTCATGTA | 314- | | | GsmGsdCsmAsdAsmG | |
| | GC | 333_as | | | sdTsmCsdAsmUsdGsm | |
| | | | | | UsdAsmGsdC | |

TABLE 2
Antisense oligonucleotides targeting the 3′ UTR

| SEQ | | | Off- | | | |
|---|---|---|---|---|---|---|
| ID | NUCLEOBASE | Oligo | Target | AG | Exemplary Modified | SEQ ID |
| NO | SEQUENCE | Name | Score | Target | Sequence | NO |
| 85 | TCACTTACAG | NM_00524 | 2 | −8.3 | mUsdCsmAsdCsmUsd | 469 |
| | TCTGGTCCCA | 9.5_1970- | | | TsmAsdCsmAsdGsmU | |
| | | 1989_as | | | sdCsmUsdGsmGsdTs | |
| | | | | | mCsdCsmCsdA | |
| 86 | TTCACTTACA | NM_00524 | 2 | −7.6 | mUsdTsmCsdAsmCsd | 470 |
| | GTCTGGTCCC | 9.5_1971- | | | TsmUsdAsmCsdAsmG | |
| | | 1990_as | | | sdTsmCsdTsmGsdGsm | |
| | | | | | UsdCsmCsdC | |
| 87 | ACGTTCACTT | NM_00524 | 3 | −8 | mAsdCsmGsdTsmUsd | 471 |
| | ACAGTCTGG | 9.5_1974- | | | CsmAsdCsmUsdTsmA | |
| | T | 1993_as | | | sdCsmAsdGsmUsdCs | |
| | | | | | mUsdGsmGsdT | |
| 88 | GTGTAAAAC | NM_00524 | 2 | −7.4 | mGsdTsmGsdTsmAsd | 472 |
| | GTTCACTTAC | 9.5_1981- | | | AsmAsdAsmCsdGsmU | |
| | A | 2000_as | | | sdTsmCsdAsmCsdTsm | |
| | | | | | UsdAsmCsdA | |
| 89 | TGTGTAAAA | NM_00524 | 2 | −8.8 | mUsdGsmUsdGsmUsd | 473 |
| | CGTTCACTTA | 9.5_1982- | | | AsmAsdAsmAsdCsmG | |
| | C | 2001_as | | | sdTsmUsdCsmAsdCsm | |
| | | | | | UsdTsmAsdC | |
| 90 | GTGTGTAAA | NM_00524 | 2 | −8 | mGsdTsmGsdTsmGsd | 474 |
| | ACGTTCACTT | 9.5_1983- | | | TsmAsdAsmAsdAsmC | |
| | A | 2002_as | | | sdGsmUsdTsmCsdAs | |
| | | | | | mCsdTsmUsdA | |
| 91 | TGTGTGTAA | NM_00524 | 2 | −7 | mUsdGsmUsdGsmUsd | 475 |
| | AACGTTCACT | 9.5_1984- | | | GsmUsdAsmAsdAsm | |
| | T | 2003_as | | | AsdCsmGsdTsmUsdCs | |
| | | | | | mAsdCsmUsdT | |
| 92 | TGCAAATGT | NM_00524 | 2 | −6.9 | mUsdGsmCsdAsmAsd | 476 |
| | GTGTAAAAC | 9.5_1990- | | | AsmUsdGsmUsdGsm | |
| | GT | 2009_as | | | UsdGsmUsdAsmAsdA | |
| | | | | | smAsdCsmGsdT | |
| 93 | ATGCAAATG | NM_00524 | 2 | −6.6 | mAsdTsmGsdCsmAsd | 477 |
| | TGTGTAAAA | 9.5_1991- | | | AsmAsdTsmGsdTsmG | |
| | CG | 2010_as | | | sdTsmGsdTsmAsdAsm | |
| | | | | | AsdAsmCsdG | |
| 94 | AATGCAAAT | NM_00524 | 2 | −8.1 | mAsdAsmUsdGsmCsd | 478 |
| | GTGTGTAAA | 9.5_1992- | | | AsmAsdAsmUsdGsm | |
| | AC | 2011_as | | | UsdGsmUsdGsmUsdA | |
| | | | | | smAsdAsmAsdC | |
| 95 | CAATGCAAA | NM_00524 | 2 | −11 | mCsdAsmAsdTsmGsd | 479 |
| | TGTGTGTAA | 9.5_1993- | | | CsmAsdAsmAsdTsmG | |
| | AA | 2012_as | | | sdTsmGsdTsmGsdTsm | |
| | | | | | AsdAsmAsdA | |
| 96 | TTTACAATGC | NM_00524 | 2 | −15.1 | mUsdTsmUsdAsmCsd | 480 |
| | AAATGTGTG | 9.5_1997- | | | AsmAsdTsmGsdCsmA | |
| | T | 2016_as | | | sdAsmAsdTsmGsdTsm | |
| | | | | | GsdTsmGsdT | |
| 97 | AAATACCTG | NM_00524 | 2 | −10 | mAsdAsmAsdTsmAsd | 481 |
| | GACTTATTTT | 9.5_2027- | | | CsmCsdTsmGsdGsmA | |
| | T | 2046_as | | | sdCsmUsdTsmAsdTsm | |
| | | | | | UsdTsmUsdT | |
| 98 | AAAATACCT | NM_00524 | 2 | −9.4 | mAsdAsmAsdAsmUsd | 482 |
| | GGACTTATTT | 9.5_2028- | | | AsmCsdCsmUsdGsmG | |
| | T | 2047_as | | | sdAsmCsdTsmUsdAs | |
| | | | | | mUsdTsmUsdT | |
| 99 | AAAAATACC | NM_00524 | 2 | −7.9 | mAsdAsmAsdAsmAsd | 483 |
| | TGGACTTATT | 9.5_2029- | | | TsmAsdCsmCsdTsmG | |
| | T | 2048_as | | | sdGsmAsdCsmUsdTs | |
| | | | | | mAsdTsmUsdT | |
| 100 | AACGTACAG | NM_00524 | 2 | −11.2 | mAsdAsmCsdGsmUsd | 484 |
| | AAATGGGAG | 9.5_2061- | | | AsmCsdAsmGsdAsmA | |
| | GG | 2080_as | | | sdAsmUsdGsmGsdGs | |
| | | | | | mAsdGsmGsdG | |
| 101 | AAACGTACA | NM_00524 | 2 | −11.6 | mAsdAsmAsdCsmGsd | 485 |
| | GAAATGGGA | 9.5_2062- | | | TsmAsdCsmAsdGsmA | |
| | GG | 2081_as | | | sdAsmAsdTsmGsdGs | |
| | | | | | mGsdAsmGsdG | |
| 102 | CAAACGTAC | NM_00524 | 2 | −11.1 | mCsdAsmAsdAsmCsd | 486 |
| | AGAAATGGG | 9.5_2063- | | | GsmUsdAsmCsdAsmG | |
| | AG | 2082_as | | | sdAsmAsdAsmUsdGs | |
| | | | | | mGsdGsmAsdG | |
| 103 | ACAAACGTA | NM_00524 | 2 | −9.7 | mAsdCsmAsdAsmAsd | 487 |
| | CAGAAATGG | 9.5_2064- | | | CsmGsdTsmAsdCsmA | |
| | GA | 2083_as | | | sdGsmAsdAsmAsdTs | |
| | | | | | mGsdGsmGsdA | |
| 104 | AACAAACGT | NM_00524 | 2 | −10 | mAsdAsmCsdAsmAsd | 488 |
| | ACAGAAATG | 9.5_2065- | | | AsmCsdGsmUsdAsmC | |
| | GG | 2084_as | | | sdAsmGsdAsmAsdAs | |
| | | | | | mUsdGsmGsdG | |
| 105 | GAACAAACG | NM_00524 | 2 | −6.8 | mGsdAsmAsdCsmAsd | 489 |
| | TACAGAAAT | 9.5_2066- | | | AsmAsdCsmGsdTsmA | |
| | GG | 2085_as | | | sdCsmAsdGsmAsdAs | |
| | | | | | mAsdTsmGsdG | |
| 106 | CACTCCACA | NM_00524 | 2 | −17.2 | mCsdAsmCsdTsmCsd | 490 |
| | CCTTGTTAGA | 9.5_2107- | | | CsmAsdCsmAsdCsmC | |
| | A | 2126_as | | | sdTsmUsdGsmUsdTsm | |
| | | | | | AsdGsmAsdA | |
| 107 | ACACTCCAC | NM_00524 | 2 | −18.1 | mAsdCsmAsdCsmUsd | 491 |
| | ACCTTGTTAG | 9.5_2108- | | | CsmCsdAsmCsdAsmC | |
| | A | 2127_as | | | sdCsmUsdTsmGsdTsm | |
| | | | | | UsdAsmGsdA | |
| 108 | GACACTCCA | NM_00524 | 2 | −18.1 | mGsdAsmCsdAsmCsd | 492 |
| | CACCTTGTTA | 9.5_2109- | | | TsmCsdCsmAsdCsmA | |
| | G | 2128_as | | | sdCsmCsdTsmUsdGsm | |
| | | | | | UsdTsmAsdG | |
| 109 | TCGCTGACA | NM_00524 | 2 | −10.5 | mUsdCsmGsdCsmUsd | 493 |
| | CTCCACACCT | 9.5_2114- | | | GsmAsdCsmAsdCsmU | |
| | T | 2133_as | | | sdCsmCsdAsmCsdAs | |
| | | | | | mCsdCsmUsdT | |
| 110 | GTATTCTCCC | NM_00524 | 2 | −7.2 | mGsdTsmAsdTsmUsd | 49 |
| | CACATTGCA | 9.5_2135- | | | CsmUsdCsmCsdCsmC | |
| | C | 2154_as | | | sdAsmCsdAsmUsdTs | |
| | | | | | mGsdCsmAsdC | |
| 111 | TGTATTCTCC | NM_00524 | 2 | −10 | mUsdGsmUsdAsmUsd | 495 |
| | CCACATTGC | 9.5_2136- | | | TsmCsdTsmCsdCsmCs | |
| | A | 2155_as | | | dCsmAsdCsmAsdTsm | |
| | | | | | UsdGsmCsdA | |
| 112 | ATGTATTCTC | NM_00524 | 2 | −10.5 | mAsdTsmGsdTsmAsd | 496 |
| | CCCACATTGC | 9.5_2137- | | | TsmUsdCsmUsdCsmC | |
| | | 2156_as | | | sdCsmCsdAsmCsdAs | |
| | | | | | mUsdTsmGsdC | |
| 113 | ACAATGTATT | NM_00524 | 2 | −6.3 | mAsdCsmAsdAsmUsd | 497 |
| | CTCCCCACAT | 9.5_2140- | | | GsmUsdAsmUsdTsmC | |
| | | 2159_as | | | sdTsmCsdCsmCsdCsm | |
| | | | | | AsdCsmAsdT | |
| 114 | TTGACTTCCA | NM_00524 | 2 | −8.1 | mUsdTsmGsdAsmCsd | 498 |
| | AACCTTATAT | 9.5_2163- | | | TsmUsdCsmCsdAsmA | |
| | | 2182_as | | | sdAsmCsdCsmUsdTsm | |
| | | | | | AsdTsmAsdT | |
| 115 | TTTGACTTCC | NM_00524 | 2 | −7.2 | mUsdTsmUsdGsmAsd | 499 |
| | AAACCTTAT | 9.5_2164- | | | CsmUsdTsmCsdCsmA | |
| | A | 2183_as | | | sdAsmAsdCsmCsdTsm | |
| | | | | | UsdAsmUsdA | |
| 116 | CTACTATAAT | NM_00524 | 2 | −7.4 | mCsdTsmAsdCsmUsd | 500 |
| | TTGACTTCCA | 9.5_2173- | | | AsmUsdAsmAsdTsmU | |
| | | 2192_as | | | sdTsmGsdAsmCsdTsm | |
| | | | | | UsdCsmCsdA | |
| 117 | TCTACTATAA | NM_00524 | 2 | −8 | mUsdCsmUsdAsmCsd | 501 |
| | TTTGACTTCC | 9.5_2174- | | | TsmAsdTsmAsdAsmU | |
| | | 2193_as | | | sdTsmUsdGsmAsdCs | |
| | | | | | mUsdTsmCsdC | |
| 118 | TTCTACTATA | NM_00524 | 2 | −9 | mUsdTsmCsdTsmAsd | 502 |
| | ATTTGACTTC | 9.5_2175- | | | CsmUsdAsmUsdAsmA | |
| | | 2194_as | | | sdTsmUsdTsmGsdAsm | |
| | | | | | CsdTsmUsdC | |
| 119 | CATTCTACTA | NM_00524 | 2 | −7.9 | mCsdAsmUsdTsmCsd | 503 |
| | TAATTTGACT | 9.5_2177- | | | TsmAsdCsmUsdAsmU | |
| | | 2196_as | | | sdAsmAsdTsmUsdTsm | |
| | | | | | GsdAsmCsdT | |
| 120 | ACATTCTACT | NM_00524 | 2 | −9.9 | mAsdCsmAsdTsmUsd | 504 |
| | ATAATTTGAC | 9.5_2178- | | | CsmUsdAsmCsdTsmA | |
| | | 2197_as | | | sdTsmAsdAsmUsdTsm | |
| | | | | | UsdGsmAsdC | |
| 121 | GATACACAT | NM_00524 | 2 | −10.1 | mGsdAsmUsdAsmCsd | 505 |
| | TCTACTATAA | 9.5_2183- | | | AsmCsdAsmUsdTsmC | |
| | T | 2202_as | | | sdTsmAsdCsmUsdAs | |
| | | | | | mUsdAsmAsdT | |
| 122 | AGATACACA | NM_00524 | 2 | −10.1 | mAsdGsmAsdTsmAsd | 506 |
| | TTCTACTATA | 9.5_2184- | | | CsmAsdCsmAsdTsmU | |
| | A | 2203_as | | | sdCsmUsdAsmCsdTsm | |
| | | | | | AsdTsmAsdA | |
| 123 | TAGATACAC | NM_00524 | 2 | −10.5 | mUsdAsmGsdAsmUsd | 507 |
| | ATTCTACTAT | 9.5_2185- | | | AsmCsdAsmCsdAsmU | |
| | A | 2204_as | | | sdTsmCsdTsmAsdCsm | |
| | | | | | UsdAsmUsdA | |
| 124 | TTAGATACA | NM_00524 | 2 | −10.9 | mUsdTsmAsdGsmAsd | 508 |
| | CATTCTACTA | 9.5_2186- | | | TsmAsdCsmAsdCsmA | |
| | T | 2205_as | | | sdTsmUsdCsmUsdAs | |
| | | | | | mCsdTsmAsdT | |
| 125 | TTTAGATACA | NM_00524 | 2 | −11 | mUsdTsmUsdAsmGsd | 509 |
| | CATTCTACTA | 9.5_2187- | | | AsmUsdAsmCsdAsmC | |
| | | 2206_as | | | sdAsmUsdTsmCsdTsm | |
| | | | | | AsdCsmUsdA | |
| 126 | ATTTAGATAC | NM_00524 | 2 | −11.3 | mAsdTsmUsdTsmAsd | 510 |
| | ACATTCTACT | 9.5_2188- | | | GsmAsdTsmAsdCsmA | |
| | | 2207_as | | | sdCsmAsdTsmUsdCsm | |
| | | | | | UsdAsmCsdT | |
| 127 | TATTTAGATA | NM_00524 | 2 | −6.7 | mUsdAsmUsdTsmUsd | 511 |
| | CACATTCTAC | 9.5_2189- | | | AsmGsdAsmUsdAsmC | |
| | | 2208_as | | | sdAsmCsdAsmUsdTs | |
| | | | | | mCsdTsmAsdC | |
| 128 | CTATTTAGAT | NM_00524 | 2 | −10.2 | mCsdTsmAsdTsmUsd | 512 |
| | ACACATTCTA | 9.5_2190- | | | TsmAsdGsmAsdTsmA | |
| | | 2209_as | | | sdCsmAsdCsmAsdTsm | |
| | | | | | UsdCsmUsdA | |
| 129 | CACTATTTAG | NM_00524 | 2 | −13.6 | mCsdAsmCsdTsmAsd | 513 |
| | ATACACATTC | 9.5_2192- | | | TsmUsdTsmAsdGsmA | |
| | | 2211_as | | | sdTsmAsdCsmAsdCsm | |
| | | | | | AsdTsmUsdC | |
| 130 | GTCACTATTT | NM_00524 | 2 | −14.7 | mGsdTsmCsdAsmCsd | 514 |
| | AGATACACA | 9.5_2194- | | | TsmAsdTsmUsdTsmA | |
| | T | 2213_as | | | sdGsmAsdTsmAsdCs | |
| | | | | | mAsdCsmAsdT | |
| 131 | AGTCACTATT | NM_00524 | 2 | −13.4 | mAsdGsmUsdCsmAsd | 515 |
| | TAGATACAC | 9.5_2195- | | | CsmUsdAsmUsdTsmU | |
| | A | 2214_as | | | sdAsmGsdAsmUsdAs | |
| | | | | | mCsdAsmCsdA | |
| 132 | CAGTCACTAT | NM_00524 | 2 | −11.6 | mCsdAsmGsdTsmCsd | 516 |
| | TTAGATACA | 9.5_2196- | | | AsmCsdTsmAsdTsmU | |
| | C | 2215_as | | | sdTsmAsdGsmAsdTsm | |
| | | | | | AsdCsmAsdC | |
| 133 | AGCAGTCAC | NM_00524 | 2 | −13.1 | mAsdGsmCsdAsmGsd | 517 |
| | TATTTAGATA | 9.5_2198- | | | TsmCsdAsmCsdTsmA | |
| | C | 2217_as | | | sdTsmUsdTsmAsdGsm | |
| | | | | | AsdTsmAsdC | |
| 134 | AAGCAGTCA | NM_00524 | 2 | −12.2 | mAsdAsmGsdCsmAsd | 518 |
| | CTATTTAGAT | 9.5_2199- | | | GsmUsdCsmAsdCsmU | |
| | A | 2218_as | | | sdAsmUsdTsmUsdAs | |
| | | | | | mGsdAsmUsdA | |
| 135 | AAAGCAGTC | NM_00524 | 2 | −11.8 | mAsdAsmAsdGsmCsd | 519 |
| | ACTATTTAGA | 9.5_2200- | | | AsmGsdTsmCsdAsmC | |
| | T | 2219_as | | | sdTsmAsdTsmUsdTsm | |
| | | | | | AsdGsmAsdT | |
| 136 | CAAAGCAGT | NM_00524 | 2 | −12.5 | mCsdAsmAsdAsmGsd | 520 |
| | CACTATTTAG | 9.5_2201- | | | CsmAsdGsmUsdCsmA | |
| | A | 2220_as | | | sdCsmUsdAsmUsdTs | |
| | | | | | mUsdAsmGsdA | |
| 137 | GCAAAGCAG | NM_00524 | 2 | −13.7 | mGsdCsmAsdAsmAsd | 521 |
| | TCACTATTTA | 9.5_2202- | | | GsmCsdAsmGsdTsmC | |
| | G | 2221_as | | | sdAsmCsdTsmAsdTsm | |
| | | | | | UsdTsmAsdG | |
| 138 | GGCAAAGCA | NM_00524 | 2 | −14.9 | mGsdGsmCsdAsmAsd | 522 |
| | GTCACTATTT | 9.5_2203- | | | AsmGsdCsmAsdGsmU | |
| | A | 2222_as | | | sdCsmAsdCsmUsdAs | |
| | | | | | mUsdTsmUsdA | |
| 139 | TGGCAAAGC | NM_00524 | 2 | −15.2 | mUsdGsmGsdCsmAsd | 523 |
| | AGTCACTATT | 9.5_2204- | | | AsmAsdGsmCsdAsmG | |
| | T | 2223_as | | | sdTsmCsdAsmCsdTsm | |
| | | | | | AsdTsmUsdT | |
| 140 | AATGGCAAA | NM_00524 | 2 | −14.3 | mAsdAsmUsdGsmGsd | 524 |
| | GCAGTCACT | 9.5_2206- | | | CsmAsdAsmAsdGsmC | |
| | AT | 2225_as | | | sdAsmGsdTsmCsdAs | |
| | | | | | mCsdTsmAsdT | |
| 141 | AAATGGCAA | NM_00524 | 2 | −10.9 | mAsdAsmAsdTsmGsd | 525 |
| | AGCAGTCAC | 9.5_2207- | | | GsmCsdAsmAsdAsmG | |
| | TA | 2226_as | | | sdCsmAsdGsmUsdCs | |
| | | | | | mAsdCsmUsdA | |
| 142 | GAAATGGCA | NM_00524 | 2 | −13.2 | mGsdAsmAsdAsmUsd | 526 |
| | AAGCAGTCA | 9.5_2208- | | | GsmGsdCsmAsdAsmA | |
| | CT | 2227_as | | | sdGsmCsdAsmGsdTs | |
| | | | | | mCsdAsmCsdT | |
| 143 | AATGAAATG | NM_00524 | 2 | −10.6 | mAsdAsmUsdGsmAsd | 527 |
| | GCAAAGCAG | 9.5_2211- | | | AsmAsdTsmGsdGsmC | |
| | TC | 2230_as | | | sdAsmAsdAsmGsdCs | |
| | | | | | mAsdGsmUsdC | |
| 144 | AGGTTTGAA | NM_00524 | 2 | −6.6 | mAsdGsmGsdTsmUsd | 528 |
| | TGAAATGGC | 9.5_2218- | | | TsmGsdAsmAsdTsmG | |
| | AA | 2237_as | | | sdAsmAsdAsmUsdGs | |
| | | | | | mGsdCsmAsdA | |
| 145 | CAGGTTTGA | NM_00524 | 2 | −8 | mCsdAsmGsdGsmUsd | 529 |
| | ATGAAATGG | 9.5_2219- | | | TsmUsdGsmAsdAsmU | |
| | CA | 2238_as | | | sdGsmAsdAsmAsdTs | |
| | | | | | mGsdGsmCsdA | |
| 146 | TCAGGTTTGA | NM_00524 | 2 | −7.2 | mUsdCsmAsdGsmGsd | 530 |
| | ATGAAATGG | 9.5_2220- | | | TsmUsdTsmGsdAsmA | |
| | C | 2239_as | | | sdTsmGsdAsmAsdAs | |
| | | | | | mUsdGsmGsdC | |
| 147 | GTCAGGTTTG | NM_00524 | 2 | −6.4 | mGsdTsmCsdAsmGsd | 531 |
| | AATGAAATG | 9.5_2221- | | | GsmUsdTsmUsdGsmA | |
| | G | 2240_as | | | sdAsmUsdGsmAsdAs | |
| | | | | | mAsdTsmGsdG | |
| 148 | CTTGTCAGGT | NM_00524 | 2 | −6.2 | mCsdTsmUsdGsmUsd | 532 |
| | TTGAATGAA | 9.5_2224- | | | CsmAsdGsmGsdTsmU | |
| | A | 2243_as | | | sdTsmGsdAsmAsdTsm | |
| | | | | | GsdAsmAsdA | |
| 149 | CTTAGAGAT | NM_00524 | 2 | −7.7 | mCsdTsmUsdAsmGsd | 533 |
| | AGACTTGTC | 9.5_2236- | | | AsmGsdAsmUsdAsm | |
| | AG | 2255_as | | | GsdAsmCsdTsmUsdGs | |
| | | | | | mUsdCsmAsdG | |
| 150 | TCTTAGAGAT | NM_00524 | 2 | −11.7 | mUsdCsmUsdTsmAsd | 534 |
| | AGACTTGTC | 9.5_2237- | | | GsmAsdGsmAsdTsmA | |
| | A | 2256_as | | | sdGsmAsdCsmUsdTs | |
| | | | | | mGsdTsmCsdA | |
| 151 | CTCTTAGAG | NM_00524 | 2 | −13.4 | mCsdTsmCsdTsmUsd | 535 |
| | ATAGACTTGT | 9.5_2238- | | | AsmGsdAsmGsdAsm | |
| | C | 2257_as | | | UsdAsmGsdAsmCsdTs | |
| | | | | | mUsdGsmUsdC | |
| 152 | GCTCTTAGA | NM_00524 | 2 | −11.7 | mGsdCsmUsdCsmUsd | 536 |
| | GATAGACTT | 9.5_2239- | | | TsmAsdGsmAsdGsmA | |
| | GT | 2258_as | | | sdTsmAsdGsmAsdCs | |
| | | | | | mUsdTsmGsdT | |
| 153 | GGCTCTTAG | NM_00524 | 2 | −9 | mGsdGsmCsdTsmCsd | 537 |
| | AGATAGACT | 9.5_2240- | | | TsmUsdAsmGsdAsmG | |
| | TG | 2259_as | | | sdAsmUsdAsmGsdAs | |
| | | | | | mCsdTsmUsdG | |
| 154 | CGGCTCTTAG | NM_00524 | 3 | −8.1 | mCsdGsmGsdCsmUsd | 538 |
| | AGATAGACT | 9.5_2241- | | | CsmUsdTsmAsdGsmA | |
| | T | 2260_as | | | sdGsmAsdTsmAsdGs | |
| | | | | | mAsdCsmUsdT | |
| 155 | GCGGCTCTTA | NM_00524 | 3 | −6.8 | mGsdCsmGsdGsmCsd | 539 |
| | GAGATAGAC | 9.5_2242- | | | TsmCsdTsmUsdAsmG | |
| | T | 2261_as | | | sdAsmGsdAsmUsdAs | |
| | | | | | mGsdAsmCsdT | |
| 156 | TGGCGGCTCT | NM_00524 | 2 | −7.2 | mUsdGsmGsdCsmGsd | 540 |
| | TAGAGATAG | 9.5_2244- | | | GsmCsdTsmCsdTsmU | |
| | A | 2263_as | | | sdAsmGsdAsmGsdAs | |
| | | | | | mUsdAsmGsdA | |
| 157 | TCTGGCGGCT | NM_00524 | 2 | −8.4 | mUsdCsmUsdGsmGsd | 541 |
| | CTTAGAGAT | 9.5_2246- | | | CsmGsdGsmCsdTsmC | |
| | A | 2265_as | | | sdTsmUsdAsmGsdAs | |
| | | | | | mGsdAsmUsdA | |
| 158 | ATCTGGCGG | NM_00524 | 2 | −10 | mAsdTsmCsdTsmGsd | 542 |
| | CTCTTAGAG | 9.5_2247- | | | GsmCsdGsmGsdCsmU | |
| | AT | 2266_as | | | sdCsmUsdTsmAsdGs | |
| | | | | | mAsdGsmAsdT | |
| 159 | AATCTGGCG | NM_00524 | 2 | −9.8 | mAsdAsmUsdCsmUsd | 543 |
| | GCTCTTAGA | 9.5_2248- | | | GsmGsdCsmGsdGsmC | |
| | GA | 2267_as | | | sdTsmCsdTsmUsdAsm | |
| | | | | | GsdAsmGsdA | |
| 160 | TACTGCACA | NM_00524 | 2 | −8.1 | mUsdAsmCsdTsmGsd | 544 |
| | CATGGAAAT | 9.5_2263- | | | CsmAsdCsmAsdCsmA | |
| | CT | 2282_as | | | sdTsmGsdGsmAsdAs | |
| | | | | | mAsdTsmCsdT | |
| 161 | ATACTGCAC | NM_00524 | 2 | −9.1 | mAsdTsmAsdCsmUsd | 545 |
| | ACATGGAAA | 9.5_2264- | | | GsmCsdAsmCsdAsmC | |
| | TC | 2283_as | | | sdAsmUsdGsmGsdAs | |
| | | | | | mAsdAsmUsdC | |
| 162 | AATACTGCA | NM_00524 | 2 | −8 | mAsdAsmUsdAsmCsd | 546 |
| | CACATGGAA | 9.5_2265- | | | TsmGsdCsmAsdCsmA | |
| | AT | 2284_as | | | sdCsmAsdTsmGsdGs | |
| | | | | | mAsdAsmAsdT | |
| 163 | ATAATACTG | NM_00524 | 2 | −8.4 | mAsdTsmAsdAsmUsd | 547 |
| | CACACATGG | 9.5_2267- | | | AsmCsdTsmGsdCsmA | |
| | AA | 2286_as | | | sdCsmAsdCsmAsdTsm | |
| | | | | | GsdGsmAsdA | |
| 164 | CTTATAATAC | NM_00524 | 2 | −7.6 | mCsdTsmUsdAsmUsd | 548 |
| | TGCACACAT | 9.5_2270- | | | AsmAsdTsmAsdCsmU | |
| | G | 2289_as | | | sdGsmCsdAsmCsdAs | |
| | | | | | mCsdAsmUsdG | |
| 165 | AACTTATAAT | NM_00524 | 2 | −11.8 | mAsdAsmCsdTsmUsd | 549 |
| | ACTGCACAC | 9.5_2272- | | | AsmUsdAsmAsdTsmA | |
| | A | 2291_as | | | sdCsmUsdGsmCsdAs | |
| | | | | | mCsdAsmCsdA | |
| 166 | TAACTTATAA | NM_00524 | 3 | −12.2 | mUsdAsmAsdCsmUsd | 550 |
| | TACTGCACA | 9.5_2273- | | | TsmAsdTsmAsdAsmU | |
| | C | 2292_as | | | sdAsmCsdTsmGsdCsm | |
| | | | | | AsdCsmAsdC | |
| 167 | ATAACTTATA | NM_00524 | 2 | −15.5 | mAsdTsmAsdAsmCsd | 551 |
| | ATACTGCAC | 9.5_2274- | | | TsmUsdAsmUsdAsmA | |
| | A | 2293_as | | | sdTsmAsdCsmUsdGs | |
| | | | | | mCsdAsmCsdA | |
| 168 | GATAACTTAT | NM_00524 | 2 | −11.9 | mGsdAsmUsdAsmAsd | 552 |
| | AATACTGCA | 9.5_2275- | | | CsmUsdTsmAsdTsmA | |
| | C | 2294_as | | | sdAsmUsdAsmCsdTs | |
| | | | | | mGsdCsmAsdC | |
| 169 | TGATAACTTA | NM_00524 | 2 | −10.3 | mUsdGsmAsdTsmAsd | 553 |
| | TAATACTGC | 9.5_2276- | | | AsmCsdTsmUsdAsmU | |
| | A | 2295_as | | | sdAsmAsdTsmAsdCs | |
| | | | | | mUsdGsmCsdA | |
| 170 | ATGATAACTT | NM_00524 | 2 | −8.8 | mAsdTsmGsdAsmUsd | 554 |
| | ATAATACTG | 9.5_2277- | | | AsmAsdCsmUsdTsmA | |
| | C | 2296_as | | | sdTsmAsdAsmUsdAs | |
| | | | | | mCsdTsmGsdC | |
| 171 | GTTCCATGAT | NM_00524 | 2 | −7.1 | mGsdTsmUsdCsmCsd | 555 |
| | AACTTATAAT | 9.5_2282- | | | AsmUsdGsmAsdTsmA | |
| | | 2301_as | | | sdAsmCsdTsmUsdAs | |
| | | | | | mUsdAsmAsdT | |
| 172 | AGTTCCATG | NM_00524 | 2 | −6.6 | mAsdGsmUsdTsmCsd | 556 |
| | ATAACTTATA | 9.5_2283- | | | CsmAsdTsmGsdAsmU | |
| | A | 2302_as | | | sdAsmAsdCsmUsdTs | |
| | | | | | mAsdTsmAsdA | |
| 173 | TAGTTCCATG | NM_00524 | 2 | −6.9 | mUsdAsmGsdTsmUsd | 557 |
| | ATAACTTATA | 9.5_2284- | | | CsmCsdAsmUsdGsmA | |
| | | 2303_as | | | sdTsmAsdAsmCsdTsm | |
| | | | | | UsdAsmUsdA | |
| 174 | ATAGTTCCAT | NM_00524 | 2 | −7.2 | mAsdTsmAsdGsmUsd | 558 |
| | GATAACTTAT | 9.5_2285- | | | TsmCsdCsmAsdTsmG | |
| | | 2304_as | | | sdAsmUsdAsmAsdCs | |
| | | | | | mUsdTsmAsdT | |
| 175 | TATAGTTCCA | NM_00524 | 2 | −6.9 | mUsdAsmUsdAsmGsd | 559 |
| | TGATAACTTA | 9.5_2286- | | | TsmUsdCsmCsdAsmU | |
| | | 2305_as | | | sdGsmAsdTsmAsdAs | |
| | | | | | mCsdTsmUsdA | |
| 176 | TCTGCGTCCA | NM_00524 | 2 | −8.1 | mUsdCsmUsdGsmCsd | 560 |
| | CCATATAGTT | 9.5_2299- | | | GsmUsdCsmCsdAsmC | |
| | | 2318_as | | | sdCsmAsdTsmAsdTsm | |
| | | | | | AsdGsmUsdT | |
| 177 | GTCTGCGTCC | NM_00524 | 2 | −10.6 | mGsdTsmCsdTsmGsd | 561 |
| | ACCATATAG | 9.5_2300- | | | CsmGsdTsmCsdCsmA | |
| | T | 2319_as | | | sdCsmCsdAsmUsdAs | |
| | | | | | mUsdAsmGsdT | |
| 178 | GGTCTGCGTC | NM_00524 | 3 | −10.7 | mGsdGsmUsdCsmUsd | 562 |
| | CACCATATA | 9.5_2301- | | | GsmCsdGsmUsdCsmC | |
| | G | 2320_as | | | sdAsmCsdCsmAsdTsm | |
| | | | | | AsdTsmAsdG | |
| 179 | AGGTCTGCG | NM_00524 | 3 | −9.5 | mAsdGsmGsdTsmCsd | 563 |
| | TCCACCATAT | 9.5_2302- | | | TsmGsdCsmGsdTsmC | |
| | A | 2321_as | | | sdCsmAsdCsmCsdAs | |
| | | | | | mUsdAsmUsdA | |
| 180 | AAGGTCTGC | NM_00524 | 2 | −8.9 | mAsdAsmGsdGsmUsd | 564 |
| | GTCCACCAT | 9.5_2303- | | | CsmUsdGsmCsdGsmU | |
| | AT | 2322_as | | | sdCsmCsdAsmCsdCsm | |
| | | | | | AsdTsmAsdT | |
| 181 | TTCTCAAGGT | NM_00524 | 2 | −12.1 | mUsdTsmCsdTsmCsd | 565 |
| | CTGCGTCCAC | 9.5_2308- | | | AsmAsdGsmGsdTsmC | |
| | | 2327_as | | | sdTsmGsdCsmGsdTsm | |
| | | | | | CsdCsmAsdC | |
| 182 | GTTCTCAAG | NM_00524 | 2 | −16.1 | mGsdTsmUsdCsmUsd | 566 |
| | GTCTGCGTCC | 9.5_2309- | | | CsmAsdAsmGsdGsmU | |
| | A | 2328_as | | | sdCsmUsdGsmCsdGs | |
| | | | | | mUsdCsmCsdA | |
| 183 | TGTTCTCAAG | NM_00524 | 2 | −17.1 | mUsdGsmUsdTsmCsd | 567 |
| | GTCTGCGTCC | 9.5_2310- | | | TsmCsdAsmAsdGsmG | |
| | | 2329_as | | | sdTsmCsdTsmGsdCsm | |
| | | | | | GsdTsmCsdC | |
| 184 | TTGTTCTCAA | NM_00524 | 3 | −18.5 | mUsdTsmGsdTsmUsd | 568 |
| | GGTCTGCGTC | 9.5_2311- | | | CsmUsdCsmAsdAsmG | |
| | | 2330_as | | | sdGsmUsdCsmUsdGs | |
| | | | | | mCsdGsmUsdC | |
| 185 | GTTGTTCTCA | NM_00524 | 3 | −21.9 | mGsdTsmUsdGsmUsd | 569 |
| | AGGTCTGCG | 9.5_2312- | | | TsmCsdTsmCsdAsmA | |
| | T | 2331_as | | | sdGsmGsdTsmCsdTsm | |
| | | | | | GsdCsmGsdT | |
| 186 | GGTTGTTCTC | NM_00524 | 3 | −21.9 | mGsdGsmUsdTsmGsd | 570 |
| | AAGGTCTGC | 9.5_2313- | | | TsmUsdCsmUsdCsmA | |
| | G | 2332_as | | | sdAsmGsdGsmUsdCs | |
| | | | | | mUsdGsmCsdG | |
| 187 | AGGTTGTTCT | NM_00524 | 2 | −20 | mAsdGsmGsdTsmUsd | 571 |
| | CAAGGTCTG | 9.5_2314- | | | GsmUsdTsmCsdTsmC | |
| | C | 2333_as | | | sdAsmAsdGsmGsdTs | |
| | | | | | mCsdTsmGsdC | |
| 188 | TAGGTTGTTC | NM_00524 | 2 | −16.9 | mUsdAsmGsdGsmUsd | 572 |
| | TCAAGGTCT | 9.5_2315- | | | TsmGsdTsmUsdCsmU | |
| | G | 2334_as | | | sdCsmAsdAsmGsdGs | |
| | | | | | mUsdCsmUsdG | |
| 189 | TTAGGTTGTT | NM_00524 | 2 | −9.3 | mUsdTsmAsdGsmGsd | 573 |
| | CTCAAGGTCT | 9.5_2316- | | | TsmUsdGsmUsdTsmC | |
| | | 2335_as | | | sdTsmCsdAsmAsdGs | |
| | | | | | mGsdTsmCsdT | |
| 190 | TTTAGGTTGT | NM_00524 | 2 | −8.2 | mUsdTsmUsdAsmGsd | 574 |
| | TCTCAAGGTC | 9.5_2317- | | | GsmUsdTsmGsdTsmU | |
| | | 2336_as | | | sdCsmUsdCsmAsdAs | |
| | | | | | mGsdGsmUsdC | |
| 191 | AATTTAGGTT | NM_00524 | 2 | −6.8 | mAsdAsmUsdTsmUsd | 575 |
| | GTTCTCAAG | 9.5_2319- | | | AsmGsdGsmUsdTsmG | |
| | G | 2338_as | | | sdTsmUsdCsmUsdCsm | |
| | | | | | AsdAsmGsdG | |
| 192 | CCCATAATTT | NM_00524 | 2 | −9.9 | mCsdCsmCsdAsmUsd | 576 |
| | AGGTTGTTCT | 9.5_2324- | | | AsmAsdTsmUsdTsmA | |
| | | 2343_as | | | sdGsmGsdTsmUsdGs | |
| | | | | | mUsdTsmCsdT | |
| 193 | CCCCATAATT | NM_00524 | 2 | −12.4 | mCsdCsmCsdCsmAsd | 577 |
| | TAGGTTGTTC | 9.5_2325- | | | TsmAsdAsmUsdTsmU | |
| | | 2344_as | | | sdAsmGsdGsmUsdTs | |
| | | | | | mGsdTsmUsdC | |
| 194 | TCCCCATAAT | NM_00524 | 2 | −15.6 | mUsdCsmCsdCsmCsd | 578 |
| | TTAGGTTGTT | 9.5_2326- | | | AsmUsdAsmAsdTsmU | |
| | | 2345_as | | | sdTsmAsdGsmGsdTsm | |
| | | | | | UsdGsmUsdT | |
| 195 | CTCCCCATAA | NM_00524 | 2 | −16.4 | mCsdTsmCsdCsmCsd | 579 |
| | TTTAGGTTGT | 9.5_2327- | | | CsmAsdTsmAsdAsmU | |
| | | 2346_as | | | sdTsmUsdAsmGsdGs | |
| | | | | | mUsdTsmGsdT | |
| 196 | TCTCCCCATA | NM_00524 | 2 | −14.2 | mUsdCsmUsdCsmCsd | 580 |
| | ATTTAGGTTG | 9.5_2328- | | | CsmCsdAsmUsdAsmA | |
| | | 2347_as | | | sdTsmUsdTsmAsdGsm | |
| | | | | | GsdTsmUsdG | |
| 197 | AAATTCTCCC | NM_00524 | 2 | −11.9 | mAsdAsmAsdTsmUsd | 581 |
| | CATAATTTAG | 9.5_2332- | | | CsmUsdCsmCsdCsmC | |
| | | 2351_as | | | sdAsmUsdAsmAsdTs | |
| | | | | | mUsdTsmAsdG | |
| 198 | CAATAAATG | NM_00524 | 2 | −6 | mCsdAsmAsdTsmAsd | 582 |
| | GCCAAAATA | 9.5_2410- | | | AsmAsdTsmGsdGsmC | |
| | AT | 2429_as | | | sdCsmAsdAsmAsdAs | |
| | | | | | mUsdAsmAsdT | |
| 199 | TCTTTGGTCT | NM_00524 | 2 | −7.2 | mUsdCsmUsdTsmUsd | 583 |
| | AAAAGTAAA | 9.5_2469- | | | GsmGsdTsmCsdTsmA | |
| | C | 2488_as | | | sdAsmAsdAsmGsdTs | |
| | | | | | mAsdAsmAsdC | |
| 200 | ATCTTTGGTC | NM_00524 | 2 | −5.9 | mAsdTsmCsdTsmUsd | 584 |
| | TAAAAGTAA | 9.5_2470- | | | TsmGsdGsmUsdCsmU | |
| | A | 2489_as | | | sdAsmAsdAsmAsdGs | |
| | | | | | mUsdAsmAsdA | |
| 201 | AATCTTTGGT | NM_00524 | 2 | −7.5 | mAsdAsmUsdCsmUsd | 585 |
| | CTAAAAGTA | 9.5_2471- | | | TsmUsdGsmGsdTsmC | |
| | A | 2490_as | | | sdTsmAsdAsmAsdAs | |
| | | | | | mGsdTsmAsdA | |
| 202 | CAATCTTTGG | NM_00524 | 2 | −9.8 | mCsdAsmAsdTsmCsd | 586 |
| | TCTAAAAGT | 9.5_2472- | | | TsmUsdTsmGsdGsmU | |
| | A | 2491_as | | | sdCsmUsdAsmAsdAs | |
| | | | | | mAsdGsmUsdA | |
| 203 | TTTCTAGAAC | NM_00524 | 2 | −14.7 | mUsdTsmUsdCsmUsd | 587 |
| | CCAATCTTTG | 9.5_2483- | | | AsmGsdAsmAsdCsmC | |
| | | 2502_as | | | sdCsmAsdAsmUsdCs | |
| | | | | | mUsdTsmUsdG | |
| 204 | CATTTTCTAG | NM_00524 | 2 | −15.3 | mCsdAsmUsdTsmUsd | 588 |
| | AACCCAATC | 9.5_2486- | | | TsmCsdTsmAsdGsmA | |
| | T | 2505_as | | | sdAsmCsdCsmCsdAs | |
| | | | | | mAsdTsmCsdT | |
| 205 | GCATTTTCTA | NM_00524 | 2 | −16.2 | mGsdCsmAsdTsmUsd | 589 |
| | GAACCCAAT | 9.5_2487- | | | TsmUsdCsmUsdAsmG | |
| | C | 2506_as | | | sdAsmAsdCsmCsdCs | |
| | | | | | mAsdAsmUsdC | |
| 206 | TGCATTTTCT | NM_00524 | 2 | −14.2 | mUsdGsmCsdAsmUsd | 590 |
| | AGAACCCAA | 9.5_2488- | | | TsmUsdTsmCsdTsmA | |
| | T | 2507_as | | | sdGsmAsdAsmCsdCs | |
| | | | | | mCsdAsmAsdT | |
| 207 | GTGCATTTTC | NM_00524 | 2 | −12.6 | mGsdTsmGsdCsmAsd | 591 |
| | TAGAACCCA | 9.5_2489- | | | TsmUsdTsmUsdCsmU | |
| | A | 2508_as | | | sdAsmGsdAsmAsdCs | |
| | | | | | mCsdCsmAsdA | |
| 208 | AGTGCATTTT | NM_00524 | 2 | −12.3 | mAsdGsmUsdGsmCsd | 592 |
| | CTAGAACCC | 9.5_2490- | | | AsmUsdTsmUsdTsmC | |
| | A | 2509_as | | | sdTsmAsdGsmAsdAs | |
| | | | | | mCsdCsmCsdA | |
| 209 | CAAGTGCAT | NM_00524 | 2 | −7.2 | mCsdAsmAsdGsmUsd | 593 |
| | TTTCTAGAAC | 9.5_2492- | | | GsmCsdAsmUsdTsmU | |
| | C | 2511_as | | | sdTsmCsdTsmAsdGsm | |
| | | | | | AsdAsmCsdC | |
| 210 | CCAAGTGCA | NM_00524 | 2 | −7.6 | mCsdCsmAsdAsmGsd | 59 |
| | TTTTCTAGAA | 9.5_2493- | | | TsmGsdCsmAsdTsmU | |
| | C | 2512_as | | | sdTsmUsdCsmUsdAs | |
| | | | | | mGsdAsmAsdC | |
| 211 | ACCAAGTGC | NM_00524 | 2 | −11 | mAsdCsmCsdAsmAsd | 595 |
| | ATTTTCTAGA | 9.5_2494- | | | GsmUsdGsmCsdAsmU | |
| | A | 2513_as | | | sdTsmUsdTsmCsdTsm | |
| | | | | | AsdGsmAsdA | |
| 212 | TACCAAGTG | NM_00524 | 2 | −11.4 | mUsdAsmCsdCsmAsd | 596 |
| | CATTTTCTAG | 9.5_2495- | | | AsmGsdTsmGsdCsmA | |
| | A | 2514_as | | | sdTsmUsdTsmUsdCsm | |
| | | | | | UsdAsmGsdA | |
| 213 | ATACCAAGT | NM_00524 | 2 | −9 | mAsdTsmAsdCsmCsd | 597 |
| | GCATTTTCTA | 9.5_2496- | | | AsmAsdGsmUsdGsmC | |
| | G | 2515_as | | | sdAsmUsdTsmUsdTsm | |
| | | | | | CsdTsmAsdG | |
| 214 | TATACCAAG | NM_00524 | 2 | −11.8 | mUsdAsmUsdAsmCsd | 598 |
| | TGCATTTTCT | 9.5_2497- | | | CsmAsdAsmGsdTsmG | |
| | A | 2516_as | | | sdCsmAsdTsmUsdTsm | |
| | | | | | UsdCsmUsdA | |
| 215 | GTATACCAA | NM_00524 | 2 | −14.8 | mGsdTsmAsdTsmAsd | 599 |
| | GTGCATTTTC | 9.5_2498- | | | CsmCsdAsmAsdGsmU | |
| | T | 2517_as | | | sdGsmCsdAsmUsdTs | |
| | | | | | mUsdTsmCsdT | |
| 216 | AGTATACCA | NM_00524 | 2 | −15.2 | mAsdGsmUsdAsmUsd | 600 |
| | AGTGCATTTT | 9.5_2499- | | | AsmCsdCsmAsdAsmG | |
| | C | 2518_as | | | sdTsmGsdCsmAsdTsm | |
| | | | | | UsdTsmUsdC | |
| 217 | TAGTATACC | NM_00524 | 2 | −15.2 | mUsdAsmGsdTsmAsd | 601 |
| | AAGTGCATTT | 9.5_2500- | | | TsmAsdCsmCsdAsmA | |
| | T | 2519_as | | | sdGsmUsdGsmCsdAs | |
| | | | | | mUsdTsmUsdT | |
| 218 | TTAGTATACC | NM_00524 | 2 | −16.5 | mUsdTsmAsdGsmUsd | 602 |
| | AAGTGCATTT | 9.5_2501- | | | AsmUsdAsmCsdCsmA | |
| | | 2520_as | | | sdAsmGsdTsmGsdCs | |
| | | | | | mAsdTsmUsdT | |
| 219 | ACTTAGTATA | NM_00524 | 3 | −17.8 | mAsdCsmUsdTsmAsd | 603 |
| | CCAAGTGCA | 9.5_2503- | | | GsmUsdAsmUsdAsmC | |
| | T | 2522_as | | | sdCsmAsdAsmGsdTs | |
| | | | | | mGsdCsmAsdT | |
| 220 | TACTTAGTAT | NM_00524 | 3 | −17.3 | mUsdAsmCsdTsmUsd | 604 |
| | ACCAAGTGC | 9.5_2504- | | | AsmGsdTsmAsdTsmA | |
| | A | 2523_as | | | sdCsmCsdAsmAsdGs | |
| | | | | | mUsdGsmCsdA | |
| 221 | ATACTTAGTA | NM_00524 | 2 | −16.6 | mAsdTsmAsdCsmUsd | 605 |
| | TACCAAGTG | 9.5_2505- | | | TsmAsdGsmUsdAsmU | |
| | C | 2524_as | | | sdAsmCsdCsmAsdAs | |
| | | | | | mGsdTsmGsdC | |
| 222 | AATACTTAGT | NM_00524 | 2 | −14.1 | mAsdAsmUsdAsmCsd | 606 |
| | ATACCAAGT | 9.5_2506- | | | TsmUsdAsmGsdTsmA | |
| | G | 2525_as | | | sdTsmAsdCsmCsdAsm | |
| | | | | | AsdGsmUsdG | |
| 223 | GTTTTAATAC | NM_00524 | 2 | −14.4 | mGsdTsmUsdTsmUsd | 607 |
| | TTAGTATACC | 9.5_2511- | | | AsmAsdTsmAsdCsmU | |
| | | 2530_as | | | sdTsmAsdGsmUsdAs | |
| | | | | | mUsdAsmCsdC | |
| 224 | AGTGTTGCC | NM_00524 | 2 | −8.2 | mAsdGsmUsdGsmUsd | 608 |
| | AACTGAAAC | 9.5_2546- | | | TsmGsdCsmCsdAsmA | |
| | AA | 2565_as | | | sdCsmUsdGsmAsdAs | |
| | | | | | mAsdCsmAsdA | |
| 225 | CAATTGAAT | NM_00524 | 2 | −13.6 | mCsdAsmAsdTsmUsd | 609 |
| | GGGCAGTGT | 9.5_2559- | | | GsmAsdAsmUsdGsm | |
| | TG | 2578_as | | | GsdGsmCsdAsmGsdTs | |
| | | | | | mGsdTsmUsdG | |
| 226 | TCAATTGAAT | NM_00524 | 2 | −13.5 | mUsdCsmAsdAsmUsd | 610 |
| | GGGCAGTGT | 9.5_2560- | | | TsmGsdAsmAsdTsmG | |
| | T | 2579_as | | | sdGsmGsdCsmAsdGs | |
| | | | | | mUsdGsmUsdT | |
| 227 | TTCAATTGAA | NM_00524 | 2 | −13 | mUsdTsmCsdAsmAsd | 611 |
| | TGGGCAGTG | 9.5_2561- | | | TsmUsdGsmAsdAsmU | |
| | T | 2580_as | | | sdGsmGsdGsmCsdAs | |
| | | | | | mGsdTsmGsdT | |
| 228 | TGAAGGCAA | NM_00524 | 2 | −7.3 | mUsdGsmAsdAsmGsd | 612 |
| | TCGTTAATTT | 9.5_2593- | | | GsmCsdAsmAsdTsmC | |
| | T | 2612_as | | | sdGsmUsdTsmAsdAs | |
| | | | | | mUsdTsmUsdT | |
| 229 | CTGAAGGCA | NM_00524 | 2 | −9 | mCsdTsmGsdAsmAsd | 613 |
| | ATCGTTAATT | 9.5_2594- | | | GsmGsdCsmAsdAsmU | |
| | T | 2613_as | | | sdCsmGsdTsmUsdAs | |
| | | | | | mAsdTsmUsdT | |
| 230 | ACTGAAGGC | NM_00524 | 3 | −10 | mAsdCsmUsdGsmAsd | 614 |
| | AATCGTTAAT | 9.5_2595- | | | AsmGsdGsmCsdAsmA | |
| | T | 2614_as | | | sdTsmCsdGsmUsdTsm | |
| | | | | | AsdAsmUsdT | |
| 231 | AACTGAAGG | NM_00524 | 2 | −10.2 | mAsdAsmCsdTsmGsd | 615 |
| | CAATCGTTA | 9.5_2596- | | | AsmAsdGsmGsdCsmA | |
| | AT | 2615_as | | | sdAsmUsdCsmGsdTs | |
| | | | | | mUsdAsmAsdT | |
| 232 | AAACTGAAG | NM_00524 | 2 | −8.9 | mAsdAsmAsdCsmUsd | 616 |
| | GCAATCGTT | 9.5_2597- | | | GsmAsdAsmGsdGsmC | |
| | AA | 2616_as | | | sdAsmAsdTsmCsdGs | |
| | | | | | mUsdTsmAsdA | |
| 233 | CAAACTGAA | NM_00524 | 2 | −7.8 | mCsdAsmAsdAsmCsd | 617 |
| | GGCAATCGT | 9.5_2598- | | | TsmGsdAsmAsdGsmG | |
| | TA | 2617_as | | | sdCsmAsdAsmUsdCs | |
| | | | | | mGsdTsmUsdA | |
| 234 | ACAAACTGA | NM_00524 | 2 | −8.2 | mAsdCsmAsdAsmAsd | 618 |
| | AGGCAATCG | 9.5_2599- | | | CsmUsdGsmAsdAsmG | |
| | TT | 2618_as | | | sdGsmCsdAsmAsdTs | |
| | | | | | mCsdGsmUsdT | |
| 235 | ACACAAACT | NM_00524 | 2 | −7.2 | mAsdCsmAsdCsmAsd | 619 |
| | GAAGGCAAT | 9.5_2601- | | | AsmAsdCsmUsdGsmA | |
| | CG | 2620_as | | | sdAsmGsdGsmCsdAs | |
| | | | | | mAsdTsmCsdG | |
| 236 | GTGACCACA | NM_00524 | 2 | −6.9 | mGsdTsmGsdAsmCsd | 620 |
| | TACATCAAA | 9.5_2628- | | | CsmAsdCsmAsdTsmA | |
| | AT | 2647_as | | | sdCsmAsdTsmCsdAsm | |
| | | | | | AsdAsmAsdT | |
| 237 | TTAGTGACC | NM_00524 | 2 | −5.9 | mUsdTsmAsdGsmUsd | 621 |
| | ACATACATC | 9.5_2631- | | | GsmAsdCsmCsdAsmC | |
| | AA | 2650_as | | | sdAsmUsdAsmCsdAs | |
| | | | | | mUsdCsmAsdA | |
| 238 | TTTACCTATA | NM_00524 | 2 | −7.2 | mUsdTsmUsdAsmCsd | 622 |
| | AGTACAATA | 9.5_2694- | | | CsmUsdAsmUsdAsmA | |
| | G | 2713_as | | | sdGsmUsdAsmCsdAs | |
| | | | | | mAsdTsmAsdG | |
| 239 | GTTTACCTAT | NM_00524 | 2 | −8.4 | mGsdTsmUsdTsmAsd | 623 |
| | AAGTACAAT | 9.5_2695- | | | CsmCsdTsmAsdTsmA | |
| | A | 2714_as | | | sdAsmGsdTsmAsdCs | |
| | | | | | mAsdAsmUsdA | |
| 240 | GGTTTACCTA | NM_00524 | 2 | −9.9 | mGsdGsmUsdTsmUsd | 624 |
| | TAAGTACAA | 9.5_2696- | | | AsmCsdCsmUsdAsmU | |
| | T | 2715_as | | | sdAsmAsdGsmUsdAs | |
| | | | | | mCsdAsmAsdT | |
| 241 | ACATATTTGC | NM_00524 | 2 | −6.7 | mAsdCsmAsdTsmAsd | 625 |
| | AAGGTTTAC | 9.5_2708- | | | TsmUsdTsmGsdCsmA | |
| | C | 2727_as | | | sdAsmGsdGsmUsdTs | |
| | | | | | mUsdAsmCsdC | |
| 242 | TACATATTTG | NM_00524 | 2 | −7.6 | mUsdAsmCsdAsmUsd | 626 |
| | CAAGGTTTA | 9.5_2709- | | | AsmUsdTsmUsdGsmC | |
| | C | 2728_as | | | sdAsmAsdGsmGsdTs | |
| | | | | | mUsdTsmAsdC | |
| 243 | TTACATATTT | NM_00524 | 2 | −10.4 | mUsdTsmAsdCsmAsd | 627 |
| | GCAAGGTTT | 9.5_2710- | | | TsmAsdTsmUsdTsmG | |
| | A | 2729_as | | | sdCsmAsdAsmGsdGs | |
| | | | | | mUsdTsmUsdA | |
| 244 | GTTACATATT | NM_00524 | 2 | −13.4 | mGsdTsmUsdAsmCsd | 628 |
| | TGCAAGGTTT | 9.5_2711- | | | AsmUsdAsmUsdTsmU | |
| | | 2730_as | | | sdGsmCsdAsmAsdGs | |
| | | | | | mGsdTsmUsdT | |
| 245 | GGTTACATAT | NM_00524 | 2 | −14.1 | mGsdGsmUsdTsmAsd | 629 |
| | TTGCAAGGTT | 9.5_2712- | | | CsmAsdTsmAsdTsmU | |
| | | 2731_as | | | sdTsmGsdCsmAsdAs | |
| | | | | | mGsdGsmUsdT | |
| 246 | AGGTTACAT | NM_00524 | 2 | −13 | mAsdGsmGsdTsmUsd | 630 |
| | ATTTGCAAG | 9.5_2713- | | | AsmCsdAsmUsdAsmU | |
| | GT | 2732_as | | | sdTsmUsdGsmCsdAs | |
| | | | | | mAsdGsmGsdT | |
| 247 | CAGGTTACA | NM_00524 | 2 | −8.7 | mCsdAsmGsdGsmUsd | 631 |
| | TATTTGCAAG | 9.5_2714- | | | TsmAsdCsmAsdTsmA | |
| | G | 2733_as | | | sdTsmUsdTsmGsdCsm | |
| | | | | | AsdAsmGsdG | |
| 248 | ACAGGTTAC | NM_00524 | 2 | −7.1 | mAsdCsmAsdGsmGsd | 632 |
| | ATATTTGCAA | 9.5_2715- | | | TsmUsdAsmCsdAsmU | |
| | G | 2734_as | | | sdAsmUsdTsmUsdGs | |
| | | | | | mCsdAsmAsdG | |
| 249 | ACACAGGTT | NM_00524 | 2 | −14.1 | mAsdCsmAsdCsmAsd | 633 |
| | ACATATTTGC | 9.5_2717- | | | GsmGsdTsmUsdAsmC | |
| | A | 2736_as | | | sdAsmUsdAsmUsdTs | |
| | | | | | mUsdGsmCsdA | |
| 250 | AACACAGGT | NM_00524 | 2 | −10.4 | mAsdAsmCsdAsmCsd | 634 |
| | TACATATTTG | 9.5_2718- | | | AsmGsdGsmUsdTsmA | |
| | C | 2737_as | | | sdCsmAsdTsmAsdTsm | |
| | | | | | UsdTsmGsdC | |
| 251 | GCAACACAG | NM_00524 | 2 | −6.2 | mGsdCsmAsdAsmCsd | 635 |
| | GTTACATATT | 9.5_2720- | | | AsmCsdAsmGsdGsmU | |
| | T | 2739_as | | | sdTsmAsdCsmAsdTsm | |
| | | | | | AsdTsmUsdT | |
| 252 | GCGCAACAC | NM_00524 | 3 | −9.2 | mGsdCsmGsdCsmAsd | 636 |
| | AGGTTACAT | 9.5_2722- | | | AsmCsdAsmCsdAsmG | |
| | AT | 2741_as | | | sdGsmUsdTsmAsdCs | |
| | | | | | mAsdTsmAsdT | |
| 253 | TGCGCAACA | NM_00524 | 2 | −9.1 | mUsdGsmCsdGsmCsd | 637 |
| | CAGGTTACA | 9.5_2723- | | | AsmAsdCsmAsdCsmA | |
| | TA | 2742_as | | | sdGsmGsdTsmUsdAs | |
| | | | | | mCsdAsmUsdA | |
| 254 | TTGCGCAAC | NM_00524 | 2 | −8.8 | mUsdTsmGsdCsmGsd | 638 |
| | ACAGGTTAC | 9.5_2724- | | | CsmAsdAsmCsdAsmC | |
| | AT | 2743_as | | | sdAsmGsdGsmUsdTs | |
| | | | | | mAsdCsmAsdT | |
| 255 | TTTGCGCAAC | NM_00524 | 2 | −8.8 | mUsdTsmUsdGsmCsd | 639 |
| | ACAGGTTAC | 9.5_2725- | | | GsmCsdAsmAsdCsmA | |
| | A | 2744_as | | | sdCsmAsdGsmGsdTs | |
| | | | | | mUsdAsmCsdA | |
| 256 | CATTTGCGCA | NM_00524 | 2 | −7.3 | mCsdAsmUsdTsmUsd | 640 |
| | ACACAGGTT | 9.5_2727- | | | GsmCsdGsmCsdAsmA | |
| | A | 2746_as | | | sdCsmAsdCsmAsdGs | |
| | | | | | mGsdTsmUsdA | |
| 257 | ACTCAAATTT | NM_00524 | 2 | −6.1 | mAsdCsmUsdCsmAsd | 641 |
| | ATGCGGCAT | 9.5_2743- | | | AsmAsdTsmUsdTsmA | |
| | T | 2762_as | | | sdTsmGsdCsmGsdGs | |
| | | | | | mCsdAsmUsdT | |
| 258 | ATCACTCAA | NM_00524 | 3 | −8.3 | mAsdTsmCsdAsmCsd | 642 |
| | ATTTATGCGG | 9.5_2746- | | | TsmCsdAsmAsdAsmU | |
| | C | 2765_as | | | sdTsmUsdAsmUsdGs | |
| | | | | | mCsdGsmGsdC | |
| 259 | ACATTAACA | NM_00524 | 2 | −7.7 | mAsdCsmAsdTsmUsd | 643 |
| | ATCACTCAA | 9.5_2755- | | | AsmAsdCsmAsdAsmU | |
| | AT | 2774_as | | | sdCsmAsdCsmUsdCs | |
| | | | | | mAsdAsmAsdT | |
| 260 | CAACATTAA | NM_00524 | 2 | −10.3 | mCsdAsmAsdCsmAsd | 644 |
| | CAATCACTC | 9.5_2757- | | | TsmUsdAsmAsdCsmA | |
| | AA | 2776_as | | | sdAsmUsdCsmAsdCs | |
| | | | | | mUsdCsmAsdA | |
| 261 | ACAACATTA | NM_00524 | 2 | −12.1 | mAsdCsmAsdAsmCsd | 645 |
| | ACAATCACT | 9.5_2758- | | | AsmUsdTsmAsdAsmC | |
| | CA | 2777_as | | | sdAsmAsdTsmCsdAs | |
| | | | | | mCsdTsmCsdA | |
| 262 | GACAACATT | NM_00524 | 2 | −14.3 | mGsdAsmCsdAsmAsd | 646 |
| | AACAATCAC | 9.5_2759- | | | CsmAsdTsmUsdAsmA | |
| | TC | 2778_as | | | sdCsmAsdAsmUsdCs | |
| | | | | | mAsdCsmUsdC | |
| 263 | AGACAACAT | NM_00524 | 2 | −11.1 | mAsdGsmAsdCsmAsd | 647 |
| | TAACAATCA | 9.5_2760- | | | AsmCsdAsmUsdTsmA | |
| | CT | 2779_as | | | sdAsmCsdAsmAsdTs | |
| | | | | | mCsdAsmCsdT | |
| 264 | ACCACAGTA | NM_00524 | 2 | −8.9 | mAsdCsmCsdAsmCsd | 648 |
| | TCACAATCA | 9.5_2788- | | | AsmGsdTsmAsdTsmC | |
| | AG | 2807_as | | | sdAsmCsdAsmAsdTs | |
| | | | | | mCsdAsmAsdG | |
| 265 | GACCACAGT | NM_00524 | 2 | −9.5 | mGsdAsmCsdCsmAsd | 649 |
| | ATCACAATC | 9.5_2789- | | | CsmAsdGsmUsdAsmU | |
| | AA | 2808_as | | | sdCsmAsdCsmAsdAs | |
| | | | | | mUsdCsmAsdA | |
| 266 | TGACCACAG | NM_00524 | 2 | −6.5 | mUsdGsmAsdCsmCsd | 650 |
| | TATCACAATC | 9.5_2790- | | | AsmCsdAsmGsdTsmA | |
| | A | 2809_as | | | sdTsmCsdAsmCsdAsm | |
| | | | | | AsdTsmCsdA | |
| 267 | ATGACCACA | NM_00524 | 2 | −6.8 | mAsdTsmGsdAsmCsd | 651 |
| | GTATCACAA | 9.5_2791- | | | CsmAsdCsmAsdGsmU | |
| | TC | 2810_as | | | sdAsmUsdCsmAsdCs | |
| | | | | | mAsdAsmUsdC | |
| 268 | CATATGACC | NM_00524 | 2 | −10.5 | mCsdAsmUsdAsmUsd | 652 |
| | ACAGTATCA | 9.5_2794- | | | GsmAsdCsmCsdAsmC | |
| | CA | 2813_as | | | sdAsmGsdTsmAsdTsm | |
| | | | | | CsdAsmCsdA | |
| 269 | GCATATGAC | NM_00524 | 2 | −11.6 | mGsdCsmAsdTsmAsd | 653 |
| | CACAGTATC | 9.5_2795- | | | TsmGsdAsmCsdCsmA | |
| | AC | 2814_as | | | sdCsmAsdGsmUsdAs | |
| | | | | | mUsdCsmAsdC | |
| 270 | GACAAACAC | NM_00524 | 2 | −10.5 | mGsdAsmCsdAsmAsd | 654 |
| | GGGCATATG | 9.5_2806- | | | AsmCsdAsmCsdGsmG | |
| | AC | 2825_as | | | sdGsmCsdAsmUsdAs | |
| | | | | | mUsdGsmAsdC | |
| 271 | TGACAAACA | NM_00524 | 2 | −8.8 | mUsdGsmAsdCsmAsd | 655 |
| | CGGGCATAT | 9.5_2807- | | | AsmAsdCsmAsdCsmG | |
| | GA | 2826_as | | | sdGsmGsdCsmAsdTs | |
| | | | | | mAsdTsmGsdA | |
| 272 | GTTCATAGTA | NM_00524 | 2 | −7.4 | mGsdTsmUsdCsmAsd | 656 |
| | AACATTTTTG | 9.5_2831- | | | TsmAsdGsmUsdAsmA | |
| | | 2850_as | | | sdAsmCsdAsmUsdTs | |
| | | | | | mUsdTsmUsdG | |
| 273 | GTGTTCATAG | NM_00524 | 2 | −8.2 | mGsdTsmGsdTsmUsd | 657 |
| | TAAACATTTT | 9.5_2833- | | | CsmAsdTsmAsdGsmU | |
| | | 2852_as | | | sdAsmAsdAsmCsdAs | |
| | | | | | mUsdTsmUsdT | |
| 274 | TGTGTTCATA | NM_00524 | 2 | −7.6 | mUsdGsmUsdGsmUsd | 658 |
| | GTAAACATTT | 9.5_2834- | | | TsmCsdAsmUsdAsmG | |
| | | 2853_as | | | sdTsmAsdAsmAsdCs | |
| | | | | | mAsdTsmUsdT | |
| 275 | TCTGTGTGTT | NM_00524 | 2 | −11.1 | mUsdCsmUsdGsmUsd | 659 |
| | CATAGTAAA | 9.5_2838- | | | GsmUsdGsmUsdTsmC | |
| | C | 2857_as | | | sdAsmUsdAsmGsdTs | |
| | | | | | mAsdAsmAsdC | |
| 276 | TTCTGTGTGT | NM_00524 | 2 | −8.5 | mUsdTsmCsdTsmGsd | 660 |
| | TCATAGTAA | 9.5_2839- | | | TsmGsdTsmGsdTsmU | |
| | A | 2858_as | | | sdCsmAsdTsmAsdGs | |
| | | | | | mUsdAsmAsdA | |
| 277 | TATTTCTGTG | NM_00524 | 2 | −6.6 | mUsdAsmUsdTsmUsd | 661 |
| | TGTTCATAGT | 9.5_2842- | | | CsmUsdGsmUsdGsmU | |
| | | 2861_as | | | sdGsmUsdTsmCsdAs | |
| | | | | | mUsdAsmGsdT | |
| 278 | GATATATAT | NM_00524 | 2 | −12.2 | mGsdAsmUsdAsmUsd | 662 |
| | GAATTTAGC | 9.5_2868- | | | AsmUsdAsmUsdGsm | |
| | CT | 2887_as | | | AsdAsmUsdTsmUsdA | |
| | | | | | smGsdCsmCsdT | |
| 279 | AGATATATA | NM_00524 | 2 | −7.7 | mAsdGsmAsdTsmAsd | 663 |
| | TGAATTTAGC | 9.5_2869- | | | TsmAsdTsmAsdTsmG | |
| | C | 2888_as | | | sdAsmAsdTsmUsdTsm | |
| | | | | | AsdGsmCsdC | |
| 280 | AGACAAAAG | NM_00524 | 2 | −9 | mAsdGsmAsdCsmAsd | 664 |
| | TATCAAGAT | 9.5_2883- | | | AsmAsdAsmGsdTsmA | |
| | AT | 2902_as | | | sdTsmCsdAsmAsdGs | |
| | | | | | mAsdTsmAsdT | |
| 281 | AGTTGATTG | NM_00524 | 2 | −7.2 | mAsdGsmUsdTsmGsd | 665 |
| | GTCTTTAAAA | 9.5_2924- | | | AsmUsdTsmGsdGsmU | |
| | A | 2943_as | | | sdCsmUsdTsmUsdAs | |
| | | | | | mAsdAsmAsdA | |
| 282 | CCCTATAAGT | NM_00524 | 2 | −6.3 | mCsdCsmCsdTsmAsd | 666 |
| | TGATTGGTCT | 9.5_2931- | | | TsmAsdAsmGsdTsmU | |
| | | 2950_as | | | sdGsmAsdTsmUsdGs | |
| | | | | | mGsdTsmCsdT | |
| 283 | AAAAAGCCT | NM_00524 | 2 | −6.5 | mAsdAsmAsdAsmAsd | 667 |
| | TTGAATTCCC | 9.5_2947- | | | GsmCsdCsmUsdTsmU | |
| | T | 2966_as | | | sdGsmAsdAsmUsdTs | |
| | | | | | mCsdCsmCsdT | |
| 284 | TAAATTTTAG | NM_00524 | 2 | −11.6 | mUsdAsmAsdAsmUsd | 668 |
| | TTTGGCTGAA | 9.5_2965- | | | TsmUsdTsmAsdGsmU | |
| | | 2984_as | | | sdTsmUsdGsmGsdCs | |
| | | | | | mUsdGsmAsdA | |
| 285 | TTAAATTTTA | NM_00524 | 2 | −12.4 | mUsdTsmAsdAsmAsd | 669 |
| | GTTTGGCTGA | 9.5_2966- | | | TsmUsdTsmUsdAsmG | |
| | | 2985_as | | | sdTsmUsdTsmGsdGsm | |
| | | | | | CsdTsmGsdA | |
| 286 | TTTAAATTTT | NM_00524 | 2 | −11.9 | mUsdTsmUsdAsmAsd | 670 |
| | AGTTTGGCTG | 9.5_2967- | | | AsmUsdTsmUsdTsmA | |
| | | 2986_as | | | sdGsmUsdTsmUsdGs | |
| | | | | | mGsdCsmUsdG | |
| 287 | GTTTAAATTT | NM_00524 | 2 | −10.4 | mGsdTsmUsdTsmAsd | 671 |
| | TAGTTTGGCT | 9.5_2968- | | | AsmAsdTsmUsdTsmU | |
| | | 2987_as | | | sdAsmGsdTsmUsdTsm | |
| | | | | | GsdGsmCsdT | |
| 288 | TTAGAGTCA | NM_00524 | 2 | −10.9 | mUsdTsmAsdGsmAsd | 672 |
| | GTTCAAATTA | 9.5_2995- | | | GsmUsdCsmAsdGsmU | |
| | A | 3014_as | | | sdTsmCsdAsmAsdAs | |
| | | | | | mUsdTsmAsdA | |
| 289 | TTTAGAGTCA | NM_00524 | 2 | −11.7 | mUsdTsmUsdAsmGsd | 673 |
| | GTTCAAATTA | 9.5_2996- | | | AsmGsdTsmCsdAsmG | |
| | | 3015_as | | | sdTsmUsdCsmAsdAs | |
| | | | | | mAsdTsmUsdA | |
| 290 | TTTTAGAGTC | NM_00524 | 2 | −14.6 | mUsdTsmUsdTsmAsd | 674 |
| | AGTTCAAATT | 9.5_2997- | | | GsmAsdGsmUsdCsmA | |
| | | 3016_as | | | sdGsmUsdTsmCsdAs | |
| | | | | | mAsdAsmUsdT | |
| 291 | TCATTTTTAG | NM_00524 | 2 | −9.8 | mUsdCsmAsdTsmUsd | 675 |
| | AGTCAGTTC | 9.5_3001- | | | TsmUsdTsmAsdGsmA | |
| | A | 3020_as | | | sdGsmUsdCsmAsdGs | |
| | | | | | mUsdTsmCsdA | |
| 292 | TTCATTTTTA | NM_00524 | 2 | −9.2 | mUsdTsmCsdAsmUsd | 676 |
| | GAGTCAGTT | 9.5_3002- | | | TsmUsdTsmUsdAsmG | |
| | C | 3021_as | | | sdAsmGsdTsmCsdAs | |
| | | | | | mGsdTsmUsdC | |
| 293 | GTTCACAAA | NM_00524 | 2 | −9 | mGsdTsmUsdCsmAsd | 677 |
| | GGGAAAAAT | 9.5_3026- | | | CsmAsdAsmAsdGsmG | |
| | AC | 3045_as | | | sdGsmAsdAsmAsdAs | |
| | | | | | mAsdTsmAsdC | |
| 294 | CTGCTCCTTG | NM_00524 | 2 | −6.5 | mCsdTsmGsdCsmUsd | 678 |
| | TAAAATTTGT | 9.5_3044- | | | CsmCsdTsmUsdGsmU | |
| | | 3063_as | | | sdAsmAsdAsmAsdTs | |
| | | | | | mUsdTsmGsdT | |
| 29.5 | GCTGCTCCTT | NM_00524 | 2 | −7.1 | mGsdCsmUsdGsmCsd | 679 |
| | GTAAAATTT | 9.5_3045- | | | TsmCsdCsmUsdTsmG | |
| | G | 3064_as | | | sdTsmAsdAsmAsdAs | |
| | | | | | mUsdTsmUsdG | |
| 296 | TGTTTATTAA | NM_00524 | 2 | −7.1 | mUsdGsmUsdTsmUsd | 680 |
| | ATAGGCTGC | 9.5_3059- | | | AsmUsdTsmAsdAsmA | |
| | T | 3078_as | | | sdTsmAsdGsmGsdCs | |
| | | | | | mUsdGsmCsdT | |
| 297 | GTGTTTATTA | NM_00524 | 2 | −7.1 | mGsdTsmGsdTsmUsd | 681 |
| | AATAGGCTG | 9.5_3060- | | | TsmAsdTsmUsdAsmA | |
| | C | 3079_as | | | sdAsmUsdAsmGsdGs | |
| | | | | | mCsdTsmGsdC | |
| 298 | TAGTGTTTAT | NM_00524 | 2 | −12.4 | mUsdAsmGsdTsmGsd | 682 |
| | TAAATAGGC | 9.5_3062- | | | TsmUsdTsmAsdTsmU | |
| | T | 3081_as | | | sdAsmAsdAsmUsdAs | |
| | | | | | mGsdGsmCsdT | |
| 299 | CTAGTGTTTA | NM_00524 | 2 | −11.4 | mCsdTsmAsdGsmUsd | 683 |
| | TTAAATAGG | 9.5_3063- | | | GsmUsdTsmUsdAsmU | |
| | C | 3082_as | | | sdTsmAsdAsmAsdTsm | |
| | | | | | AsdGsmGsdC | |
| 300 | GCTAGTGTTT | NM_00524 | 2 | −11.4 | mGsdCsmUsdAsmGsd | 684 |
| | ATTAAATAG | 9.5_3064- | | | TsmGsdTsmUsdTsmA | |
| | G | 3083_as | | | sdTsmUsdAsmAsdAs | |
| | | | | | mUsdAsmGsdG | |
| 301 | AAAGCCTAT | NM_00524 | 2 | −11.4 | mAsdAsmAsdGsmCsd | 685 |
| | ACTTTGTTTA | 9.5_3085- | | | CsmUsdAsmUsdAsmC | |
| | A | 3104_as | | | sdTsmUsdTsmGsdTsm | |
| | | | | | UsdTsmAsdA | |
| 302 | TCAGCTGAA | NM_00524 | 2 | −9.1 | mUsdCsmAsdGsmCsd | 686 |
| | AAGCCTATA | 9.5_3093- | | | TsmGsdAsmAsdAsmA | |
| | CT | 3112_as | | | sdGsmCsdCsmUsdAs | |
| | | | | | mUsdAsmCsdT | |
| 303 | ATCAGCTGA | NM_00524 | 2 | −9 | mAsdTsmCsdAsmGsd | 687 |
| | AAAGCCTAT | 9.5_3094- | | | CsmUsdGsmAsdAsmA | |
| | AC | 3113_as | | | sdAsmGsdCsmCsdTsm | |
| | | | | | AsdTsmAsdC | |
| 304 | TATCAGCTG | NM_00524 | 2 | −11.2 | mUsdAsmUsdCsmAsd | 688 |
| | AAAAGCCTA | 9.5_3095- | | | GsmCsdTsmGsdAsmA | |
| | TA | 3114_as | | | sdAsmAsdGsmCsdCs | |
| | | | | | mUsdAsmUsdA | |
| 305 | GTATCAGCT | NM_00524 | 2 | −11.2 | mGsdTsmAsdTsmCsd | 689 |
| | GAAAAGCCT | 9.5_3096- | | | AsmGsdCsmUsdGsmA | |
| | AT | 3115_as | | | sdAsmAsdAsmGsdCs | |
| | | | | | mCsdTsmAsdT | |
| 306 | GGTATCAGC | NM_00524 | 2 | −9.3 | mGsdGsmUsdAsmUsd | 690 |
| | TGAAAAGCC | 9.5_3097- | | | CsmAsdGsmCsdTsmG | |
| | TA | 3116_as | | | sdAsmAsdAsmAsdGs | |
| | | | | | mCsdCsmUsdA | |
| 307 | TGTATATCCA | NM_00524 | 2 | −5.9 | mUsdGsmUsdAsmUsd | 691 |
| | CAGAAACTT | 9.5_3119- | | | AsmUsdCsmCsdAsmC | |
| | A | 3138_as | | | sdAsmGsdAsmAsdAs | |
| | | | | | mCsdTsmUsdA | |
| 308 | CTTTTTGCTG | NM_00524 | 2 | −9.6 | mCsdTsmUsdTsmUsd | 692 |
| | TATATCCACA | 9.5_3127- | | | TsmGsdCsmUsdGsmU | |
| | | 3146_as | | | sdAsmUsdAsmUsdCs | |
| | | | | | mCsdAsmCsdA | |
| 309 | TCTTTTTGCT | NM_00524 | 2 | −8.6 | mUsdCsmUsdTsmUsd | 693 |
| | GTATATCCAC | 9.5_3128- | | | TsmUsdGsmCsdTsmG | |
| | | 3147_as | | | sdTsmAsdTsmAsdTsm | |
| | | | | | CsdCsmAsdC | |
| 310 | CTCTTTTTGC | NM_00524 | 2 | −8 | mCsdTsmCsdTsmUsd | 694 |
| | TGTATATCCA | 9.5_3129- | | | TsmUsdTsmGsdCsmU | |
| | | 3148_as | | | sdGsmUsdAsmUsdAs | |
| | | | | | mUsdCsmCsdA | |
| 311 | TCTCTTTTTG | NM_00524 | 2 | −11.8 | mUsdCsmUsdCsmUsd | 695 |
| | CTGTATATCC | 9.5_3130- | | | TsmUsdTsmUsdGsmC | |
| | | 3149_as | | | sdTsmGsdTsmAsdTsm | |
| | | | | | AsdTsmCsdC | |
| 312 | ATCTCTTTTT | NM_00524 | 2 | −12.6 | mAsdTsmCsdTsmCsd | 696 |
| | GCTGTATATC | 9.5_3131- | | | TsmUsdTsmUsdTsmG | |
| | | 3150_as | | | sdCsmUsdGsmUsdAs | |
| | | | | | mUsdAsmUsdC | |
| 313 | ATATCTCTTT | NM_00524 | 2 | −15.3 | mAsdTsmAsdTsmCsd | 697 |
| | TTGCTGTATA | 9.5_3133- | | | TsmCsdTsmUsdTsmU | |
| | | 3152_as | | | sdTsmGsdCsmUsdGs | |
| | | | | | mUsdAsmUsdA | |
| 314 | TATATCTCTT | NM_00524 | 2 | −15.3 | mUsdAsmUsdAsmUsd | 698 |
| | TTTGCTGTAT | 9.5_3134- | | | CsmUsdCsmUsdTsmU | |
| | | 3153_as | | | sdTsmUsdGsmCsdTsm | |
| | | | | | GsdTsmAsdT | |
| 315 | TTATATCTCT | NM_00524 | 2 | −15.4 | mUsdTsmAsdTsmAsd | 699 |
| | TTTTGCTGTA | 9.5_3135- | | | TsmCsdTsmCsdTsmUs | |
| | | 3154_as | | | dTsmUsdTsmGsdCsm | |
| | | | | | UsdGsmUsdA | |
| 316 | ATTATATCTC | NM_00524 | 2 | −15.7 | mAsdTsmUsdAsmUsd | 700 |
| | TTTTTGCTGT | 9.5_3136- | | | AsmUsdCsmUsdCsmU | |
| | | 3155_as | | | sdTsmUsdTsmUsdGsm | |
| | | | | | CsdTsmGsdT | |
| 317 | AATTATATCT | NM_00524 | 2 | −13.8 | mAsdAsmUsdTsmAsd | 701 |
| | CTTTTTGCTG | 9.5_3137- | | | TsmAsdTsmCsdTsmCs | |
| | | 3156_as | | | dTsmUsdTsmUsdTsm | |
| | | | | | GsdCsmUsdG | |
| 318 | GGTAAAGAG | NM_00524 | 2 | −7.8 | mGsdGsmUsdAsmAsd | 702 |
| | CTATGCACA | 9.5_3163- | | | AsmGsdAsmGsdCsmU | |
| | GA | 3182_as | | | sdAsmUsdGsmCsdAs | |
| | | | | | mCsdAsmGsdA | |
| 319 | GGGTAAAGA | NM_00524 | 3 | −9 | mGsdGsmGsdTsmAsd | 703 |
| | GCTATGCAC | 9.5_3164- | | | AsmAsdGsmAsdGsmC | |
| | AG | 3183_as | | | sdTsmAsdTsmGsdCsm | |
| | | | | | AsdCsmAsdG | |
| 320 | AGGGTAAAG | NM_00524 | 2 | −10.9 | mAsdGsmGsdGsmUsd | 704 |
| | AGCTATGCA | 9.5_3165- | | | AsmAsdAsmGsdAsm | |
| | CA | 3184_as | | | GsdCsmUsdAsmUsdG | |
| | | | | | smCsdAsmCsdA | |
| 321 | CAGGGTAAA | NM_00524 | 2 | −10.8 | mCsdAsmGsdGsmGsd | 705 |
| | GAGCTATGC | 9.5_3166- | | | TsmAsdAsmAsdGsmA | |
| | AC | 3185_as | | | sdGsmCsdTsmAsdTsm | |
| | | | | | GsdCsmAsdC | |
| 322 | ACAGGGTAA | NM_00524 | 2 | −10 | mAsdCsmAsdGsmGsd | 706 |
| | AGAGCTATG | 9.5_3167- | | | GsmUsdAsmAsdAsm | |
| | CA | 3186_as | | | GsdAsmGsdCsmUsdA | |
| | | | | | smUsdGsmCsdA | |
| 323 | AACACAGGG | NM_00524 | 2 | −7.6 | mAsdAsmCsdAsmCsd | 707 |
| | TAAAGAGCT | 9.5_3170- | | | AsmGsdGsmGsdTsmA | |
| | AT | 3189_as | | | sdAsmAsdGsmAsdGs | |
| | | | | | mCsdTsmAsdT | |
| 324 | GCCAAGCTC | NM_00524 | 2 | −5.9 | mGsdCsmCsdAsmAsd | 708 |
| | TATTAACAAT | 9.5_3240- | | | GsmCsdTsmCsdTsmA | |
| | A | 3259_as | | | sdTsmUsdAsmAsdCs | |
| | | | | | mAsdAsmUsdA | |
| 325 | TGCCAAGCT | NM_00524 | 2 | −7.4 | mUsdGsmCsdCsmAsd | 709 |
| | CTATTAACA | 9.5_3241- | | | AsmGsdCsmUsdCsmU | |
| | AT | 3260_as | | | sdAsmUsdTsmAsdAs | |
| | | | | | mCsdAsmAsdT | |
| 326 | TTGCCAAGCT | NM_00524 | 2 | −7.5 | mUsdTsmGsdCsmCsd | 710 |
| | CTATTAACA | 9.5_3242- | | | AsmAsdGsmCsdTsmC | |
| | A | 3261_as | | | sdTsmAsdTsmUsdAsm | |
| | | | | | AsdCsmAsdA | |
| 327 | TTTGCCAAGC | NM_00524 | 2 | −6.5 | mUsdTsmUsdGsmCsd | 711 |
| | TCTATTAACA | 9.5_3243- | | | CsmAsdAsmGsdCsmU | |
| | | 3262_as | | | sdCsmUsdAsmUsdTs | |
| | | | | | mAsdAsmCsdA | |
| 328 | ATAATTTGCC | NM_00524 | 2 | −9.7 | mAsdTsmAsdAsmUsd | 712 |
| | AAGCTCTATT | 9.5_3247- | | | TsmUsdGsmCsdCsmA | |
| | | 3266_as | | | sdAsmGsdCsmUsdCs | |
| | | | | | mUsdAsmUsdT | |
| 329 | TATAATTTGC | NM_00524 | 2 | −9.7 | mUsdAsmUsdAsmAsd | 713 |
| | CAAGCTCTAT | 9.5_3248- | | | TsmUsdTsmGsdCsmC | |
| | | 3267_as | | | sdAsmAsdGsmCsdTs | |
| | | | | | mCsdTsmAsdT | |
| 330 | TTATAATTTG | NM_00524 | 2 | −9.8 | mUsdTsmAsdTsmAsd | 714 |
| | CCAAGCTCT | 9.5_3249- | | | AsmUsdTsmUsdGsmC | |
| | A | 3268_as | | | sdCsmAsdAsmGsdCs | |
| | | | | | mUsdCsmUsdA | |
| 331 | ATTTATAATT | NM_00524 | 2 | −7.9 | mAsdTsmUsdTsmAsd | 715 |
| | TGCCAAGCT | 9.5_3251- | | | TsmAsdAsmUsdTsmU | |
| | C | 3270_as | | | sdGsmCsdCsmAsdAs | |
| | | | | | mGsdCsmUsdC | |
| 332 | TATTTATAAT | NM_00524 | 2 | −5.9 | mUsdAsmUsdTsmUsd | 716 |
| | TTGCCAAGCT | 9.5_3252- | | | AsmUsdAsmAsdTsmU | |
| | | 3271_as | | | sdTsmGsdCsmCsdAsm | |
| | | | | | AsdGsmCsdT | |
| 333 | TTATTTATAA | NM_00524 | 2 | −6.9 | mUsdTsmAsdTsmUsd | 717 |
| | TTTGCCAAGC | 9.5_3253- | | | TsmAsdTsmAsdAsmU | |
| | | 3272_as | | | sdTsmUsdGsmCsdCsm | |
| | | | | | AsdAsmGsdC | |
| 334 | ACTTCTATCT | NM_00524 | 2 | −7.7 | mAsdCsmUsdTsmCsd | 718 |
| | AACCATATA | 9.5_3279- | | | TsmAsdTsmCsdTsmA | |
| | C | 3298_as | | | sdAsmCsdCsmAsdTsm | |
| | | | | | AsdTsmAsdC | |
| 335 | GTCACTTCTA | NM_00524 | 2 | −10.7 | mGsdTsmCsdAsmCsd | 719 |
| | TCTAACCATA | 9.5_3282- | | | TsmUsdCsmUsdAsmU | |
| | | 3301_as | | | sdCsmUsdAsmAsdCs | |
| | | | | | mCsdAsmUsdA | |
| 336 | AGTCACTTCT | NM_00524 | 2 | −12.6 | mAsdGsmUsdCsmAsd | 720 |
| | ATCTAACCAT | 9.5_3283- | | | CsmUsdTsmCsdTsmA | |
| | | 3302_as | | | sdTsmCsdTsmAsdAsm | |
| | | | | | CsdCsmAsdT | |
| 337 | TAGTCACTTC | NM_00524 | 2 | −10 | mUsdAsmGsdTsmCsd | 721 |
| | TATCTAACCA | 9.5_3284- | | | AsmCsdTsmUsdCsmU | |
| | | 3303_as | | | sdAsmUsdCsmUsdAs | |
| | | | | | mAsdCsmCsdA | |
| 338 | ATAGTCACTT | NM_00524 | 2 | −11.6 | mAsdTsmAsdGsmUsd | 722 |
| | CTATCTAACC | 9.5_3285- | | | CsmAsdCsmUsdTsmC | |
| | | 3304_as | | | sdTsmAsdTsmCsdTsm | |
| | | | | | AsdAsmCsdC | |
| 339 | TATAGTCACT | NM_00524 | 2 | −9.3 | mUsdAsmUsdAsmGsd | 723 |
| | TCTATCTAAC | 9.5_3286- | | | TsmCsdAsmCsdTsmU | |
| | | 3305_as | | | sdCsmUsdAsmUsdCs | |
| | | | | | mUsdAsmAsdC | |
| 340 | TTATAGTCAC | NM_00524 | 2 | −7.6 | mUsdTsmAsdTsmAsd | 724 |
| | TTCTATCTAA | 9.5_3287- | | | GsmUsdCsmAsdCsmU | |
| | | 3306_as | | | sdTsmCsdTsmAsdTsm | |
| | | | | | CsdTsmAsdA | |
| 341 | ATTATAGTCA | NM_00524 | 2 | −7.5 | mAsdTsmUsdAsmUsd | 725 |
| | CTTCTATCTA | 9.5_3288- | | | AsmGsdTsmCsdAsmC | |
| | | 3307_as | | | sdTsmUsdCsmUsdAs | |
| | | | | | mUsdCsmUsdA | |
| 342 | CATTATAGTC | NM_00524 | 2 | −6.7 | mCsdAsmUsdTsmAsd | 726 |
| | ACTTCTATCT | 9.5_3289- | | | TsmAsdGsmUsdCsmA | |
| | | 3308_as | | | sdCsmUsdTsmCsdTsm | |
| | | | | | AsdTsmCsdT | |
| 343 | GCATTATAGT | NM_00524 | 2 | −9.6 | mGsdCsmAsdTsmUsd | 727 |
| | CACTTCTATC | 9.5_3290- | | | AsmUsdAsmGsdTsmC | |
| | | 3309_as | | | sdAsmCsdTsmUsdCsm | |
| | | | | | UsdAsmUsdC | |
| 344 | TGCATTATAG | NM_00524 | 2 | −9.2 | mUsdGsmCsdAsmUsd | 728 |
| | TCACTTCTAT | 9.5_3291- | | | TsmAsdTsmAsdGsmU | |
| | | 3310_as | | | sdCsmAsdCsmUsdTsm | |
| | | | | | CsdTsmAsdT | |
| 345 | GTGCATTATA | NM_00524 | 2 | −6.4 | mGsdTsmGsdCsmAsd | 729 |
| | GTCACTTCTA | 9.5_3292- | | | TsmUsdAsmUsdAsmG | |
| | | 3311_as | | | sdTsmCsdAsmCsdTsm | |
| | | | | | UsdCsmUsdA | |
| 346 | GGGCTCTGT | NM_00524 | 2 | −6 | mGsdGsmGsdCsmUsd | 730 |
| | GTGTCTATAT | 9.5_3324- | | | CsmUsdGsmUsdGsmU | |
| | A | 3343_as | | | sdGsmUsdCsmUsdAs | |
| | | | | | mUsdAsmUsdA | |
| 347 | AGGGCTCTG | NM_00524 | 2 | −7.6 | mAsdGsmGsdGsmCsd | 731 |
| | TGTGTCTATA | 9.5_3325- | | | TsmCsdTsmGsdTsmG | |
| | T | 3344_as | | | sdTsmGsdTsmCsdTsm | |
| | | | | | AsdTsmAsdT | |
| 348 | AAGGGCTCT | NM_00524 | 2 | −8 | mAsdAsmGsdGsmGsd | 732 |
| | GTGTGTCTAT | 9.5_3326- | | | CsmUsdCsmUsdGsmU | |
| | A | 3345_as | | | sdGsmUsdGsmUsdCs | |
| | | | | | mUsdAsmUsdA | |
| 349 | GAAGGGCTC | NM_00524 | 2 | −10.6 | mGsdAsmAsdGsmGsd | 733 |
| | TGTGTGTCTA | 9.5_3327- | | | GsmCsdTsmCsdTsmG | |
| | T | 3346_as | | | sdTsmGsdTsmGsdTsm | |
| | | | | | CsdTsmAsdT | |
| 350 | TGAAGGGCT | NM_00524 | 2 | −11.4 | mUsdGsmAsdAsmGsd | 734 |
| | CTGTGTGTCT | 9.5_3328- | | | GsmGsdCsmUsdCsmU | |
| | A | 3347_as | | | sdGsmUsdGsmUsdGs | |
| | | | | | mUsdCsmUsdA | |
| 351 | ACTGAAGGG | NM_00524 | 2 | −14.8 | mAsdCsmUsdGsmAsd | 735 |
| | CTCTGTGTGT | 9.5_3330- | | | AsmGsdGsmGsdCsmU | |
| | C | 3349_as | | | sdCsmUsdGsmUsdGs | |
| | | | | | mUsdGsmUsdC | |
| 352 | GAACTGAAG | NM_00524 | 2 | −9.3 | mGsdAsmAsdCsmUsd | 736 |
| | GGCTCTGTGT | 9.5_3332- | | | GsmAsdAsmGsdGsm | |
| | G | 3351_as | | | GsdCsmUsdCsmUsdG | |
| | | | | | smUsdGsmUsdG | |
| 353 | TGAACTGAA | NM_00524 | 2 | −13.1 | mUsdGsmAsdAsmCsd | 737 |
| | GGGCTCTGT | 9.5_3333- | | | TsmGsdAsmAsdGsmG | |
| | GT | 3352_as | | | sdGsmCsdTsmCsdTsm | |
| | | | | | GsdTsmGsdT | |
| 354 | CTGAACTGA | NM_00524 | 2 | −10 | mCsdTsmGsdAsmAsd | 738 |
| | AGGGCTCTG | 9.5_3334- | | | CsmUsdGsmAsdAsmG | |
| | TG | 3353_as | | | sdGsmGsdCsmUsdCs | |
| | | | | | mUsdGsmUsdG | |
| 355 | CCTGAACTG | NM_00524 | 2 | −12.5 | mCsdCsmUsdGsmAsd | 739 |
| | AAGGGCTCT | 9.5_3335- | | | AsmCsdTsmGsdAsmA | |
| | GT | 3354_as | | | sdGsmGsdGsmCsdTs | |
| | | | | | mCsdTsmGsdT | |
| 356 | AAATTGTAC | NM_00524 | 2 | −6.4 | mAsdAsmAsdTsmUsd | 740 |
| | CTGAACTGA | 9.5_3343- | | | GsmUsdAsmCsdCsmU | |
| | AG | 3362_as | | | sdGsmAsdAsmCsdTs | |
| | | | | | mGsdAsmAsdG | |
| 357 | CAAATTGTA | NM_00524 | 2 | −7.1 | mCsdAsmAsdAsmUsd | 741 |
| | CCTGAACTG | 9.5_3344- | | | TsmGsdTsmAsdCsmC | |
| | AA | 3363_as | | | sdTsmGsdAsmAsdCs | |
| | | | | | mUsdGsmAsdA | |
| 358 | GCAAATTGT | NM_00524 | 2 | −9.6 | mGsdCsmAsdAsmAsd | 742 |
| | ACCTGAACT | 9.5_3345- | | | TsmUsdGsmUsdAsmC | |
| | GA | 3364_as | | | sdCsmUsdGsmAsdAs | |
| | | | | | mCsdTsmGsdA | |
| 359 | CGCAAATTG | NM_00524 | 3 | −8.1 | mCsdGsmCsdAsmAsd | 743 |
| | TACCTGAACT | 9.5_3346- | | | AsmUsdTsmGsdTsmA | |
| | G | 3365_as | | | sdCsmCsdTsmGsdAsm | |
| | | | | | AsdCsmUsdG | |
| 360 | GCGCAAATT | NM_00524 | 3 | −6.6 | mGsdCsmGsdCsmAsd | 744 |
| | GTACCTGAA | 9.5_3347- | | | AsmAsdTsmUsdGsmU | |
| | CT | 3366_as | | | sdAsmCsdCsmUsdGs | |
| | | | | | mAsdAsmCsdT | |
| 361 | ATAAATGCT | NM_00524 | 2 | −6.4 | mAsdTsmAsdAsmAsd | 745 |
| | GACTTAGAA | 9.5_3410- | | | TsmGsdCsmUsdGsmA | |
| | AG | 3429_as | | | sdCsmUsdTsmAsdGs | |
| | | | | | mAsdAsmAsdG | |
| 362 | AAATAAATG | NM_00524 | 2 | −6.3 | mAsdAsmAsdTsmAsd | 746 |
| | CTGACTTAG | 9.5_3412- | | | AsmAsdTsmGsdCsmU | |
| | AA | 3431_as | | | sdGsmAsdCsmUsdTs | |
| | | | | | mAsdGsmAsdA | |
| 363 | AAAATAAAT | NM_00524 | 2 | −6.3 | mAsdAsmAsdAsmUsd | 747 |
| | GCTGACTTA | 9.5_3413- | | | AsmAsdAsmUsdGsmC | |
| | GA | 3432_as | | | sdTsmGsdAsmCsdTsm | |
| | | | | | UsdAsmGsdA | |
| 364 | GTGGGTAAA | NM_00524 | 2 | −6.5 | mGsdTsmGsdGsmGsd | 748 |
| | CAGCCACAA | 9.5_3430- | | | TsmAsdAsmAsdCsmA | |
| | AA | 3449_as | | | sdGsmCsdCsmAsdCs | |
| | | | | | mAsdAsmAsdA | |
| 365 | TGTGGGTAA | NM_00524 | 2 | −7.5 | mUsdGsmUsdGsmGsd | 749 |
| | ACAGCCACA | 9.5_3431- | | | GsmUsdAsmAsdAsmC | |
| | AA | 3450_as | | | sdAsmGsdCsmCsdAs | |
| | | | | | mCsdAsmAsdA | |
| 366 | ATTGTGGGT | NM_00524 | 2 | −9.7 | mAsdTsmUsdGsmUsd | 750 |
| | AAACAGCCA | 9.5_3433- | | | GsmGsdGsmUsdAsm | |
| | CA | 3452_as | | | AsdAsmCsdAsmGsdC | |
| | | | | | smCsdAsmCsdA | |
| 367 | CATTGTGGGT | NM_00524 | 2 | −7.3 | mCsdAsmUsdTsmGsd | 751 |
| | AAACAGCCA | 9.5_3434- | | | TsmGsdGsmGsdTsmA | |
| | C | 3453_as | | | sdAsmAsdCsmAsdGs | |
| | | | | | mCsdCsmAsdC | |
| 368 | TCATTGTGGG | NM_00524 | 2 | −8 | mUsdCsmAsdTsmUsd | 752 |
| | TAAACAGCC | 9.5_3435- | | | GsmUsdGsmGsdGsm | |
| | A | 3454_as | | | UsdAsmAsdAsmCsdA | |
| | | | | | smGsdCsmCsdA | |
| 369 | TTCATTGTGG | NM_00524 | 2 | −13.5 | mUsdTsmCsdAsmUsd | 753 |
| | GTAAACAGC | 9.5_3436- | | | TsmGsdTsmGsdGsmG | |
| | C | 3455_as | | | sdTsmAsdAsmAsdCs | |
| | | | | | mAsdGsmCsdC | |
| 370 | TTTCATTGTG | NM_00524 | 2 | −12.1 | mUsdTsmUsdCsmAsd | 754 |
| | GGTAAACAG | 9.5_3437- | | | TsmUsdGsmUsdGsmG | |
| | C | 3456_as | | | sdGsmUsdAsmAsdAs | |
| | | | | | mCsdAsmGsdC | |
| 371 | CTTTCATTGT | NM_00524 | 2 | −11.2 | mCsdTsmUsdTsmCsd | 755 |
| | GGGTAAACA | 9.5_3438- | | | AsmUsdTsmGsdTsmG | |
| | G | 3457_as | | | sdGsmGsdTsmAsdAs | |
| | | | | | mAsdCsmAsdG | |
| 372 | TCTTTCATTG | NM_00524 | 2 | −11.6 | mUsdCsmUsdTsmUsd | 756 |
| | TGGGTAAAC | 9.5_3439- | | | CsmAsdTsmUsdGsmU | |
| | A | 3458_as | | | sdGsmGsdGsmUsdAs | |
| | | | | | mAsdAsmCsdA | |
| 373 | CTCTTTCATT | NM_00524 | 2 | −11.8 | mCsdTsmCsdTsmUsd | 757 |
| | GTGGGTAAA | 9.5_3440- | | | TsmCsdAsmUsdTsmG | |
| | C | 3459_as | | | sdTsmGsdGsmGsdTsm | |
| | | | | | AsdAsmAsdC | |
| 374 | ACTCTTTCAT | NM_00524 | 2 | −11.8 | mAsdCsmUsdCsmUsd | 758 |
| | TGTGGGTAA | 9.5_3441- | | | TsmUsdCsmAsdTsmU | |
| | A | 3460_as | | | sdGsmUsdGsmGsdGs | |
| | | | | | mUsdAsmAsdA | |
| 375 | AACTCTTTCA | NM_00524 | 2 | −11.8 | mAsdAsmCsdTsmCsd | 759 |
| | TTGTGGGTA | 9.5_3442- | | | TsmUsdTsmCsdAsmU | |
| | A | 3461_as | | | sdTsmGsdTsmGsdGsm | |
| | | | | | GsdTsmAsdA | |
| 376 | GAACTCTTTC | NM_00524 | 2 | −12.5 | mGsdAsmAsdCsmUsd | 760 |
| | ATTGTGGGT | 9.5_3443- | | | CsmUsdTsmUsdCsmA | |
| | A | 3462_as | | | sdTsmUsdGsmUsdGs | |
| | | | | | mGsdGsmUsdA | |
| 377 | AGAACTCTTT | NM_00524 | 2 | −12.8 | mAsdGsmAsdAsmCsd | 761 |
| | CATTGTGGGT | 9.5_3444- | | | TsmCsdTsmUsdTsmCs | |
| | | 3463_as | | | dAsmUsdTsmGsdTsm | |
| | | | | | GsdGsmGsdT | |
| 378 | TAGAACTCTT | NM_00524 | 2 | −11 | mUsdAsmGsdAsmAsd | 762 |
| | TCATTGTGGG | 9.5_3445- | | | CsmUsdCsmUsdTsmU | |
| | | 3464_as | | | sdCsmAsdTsmUsdGs | |
| | | | | | mUsdGsmGsdG | |
| 379 | TTAGAACTCT | NM_00524 | 2 | −8.4 | mUsdTsmAsdGsmAsd | 763 |
| | TTCATTGTGG | 9.5_3446- | | | AsmCsdTsmCsdTsmU | |
| | | 3465_as | | | sdTsmCsdAsmUsdTsm | |
| | | | | | GsdTsmGsdG | |
| 380 | CTTTATTAGA | NM_00524 | 2 | −6.6 | mCsdTsmUsdTsmAsd | 764 |
| | ACTCTTTCAT | 9.5_3451- | | | TsmUsdAsmGsdAsmA | |
| | | 3470_as | | | sdCsmUsdCsmUsdTsm | |
| | | | | | UsdCsmAsdT | |
| 381 | ACATCTTTAT | NM_00524 | 2 | −10.7 | mAsdCsmAsdTsmCsd | 765 |
| | TAGAACTCTT | 9.5_3455- | | | TsmUsdTsmAsdTsmU | |
| | | 3474_as | | | sdAsmGsdAsmAsdCs | |
| | | | | | mUsdCsmUsdT | |
| 382 | GCACATCTTT | NM_00524 | 2 | −6.3 | mGsdCsmAsdCsmAsd | 766 |
| | ATTAGAACT | 9.5_3457- | | | TsmCsdTsmUsdTsmA | |
| | C | 3476_as | | | sdTsmUsdAsmGsdAs | |
| | | | | | mAsdCsmUsdC | |
| 383 | CAGCACATC | NM_00524 | 2 | −6.5 | mCsdAsmGsdCsmAsd | 767 |
| | TTTATTAGAA | 9.5_3459- | | | CsmAsdTsmCsdTsmU | |
| | C | 3478_as | | | sdTsmAsdTsmUsdAsm | |
| | | | | | GsdAsmAsdC | |
| 384 | TCAGCACAT | NM_00524 | 2 | −6.7 | mUsdCsmAsdGsmCsd | 768 |
| | CTTTATTAGA | 9.5_3460- | | | AsmCsdAsmUsdCsmU | |
| | A | 3479_as | | | sdTsmUsdAsmUsdTsm | |
| | | | | | AsdGsmAsdA | |

### Example 2: Cellular Modulation of FOXG1 Expression by ASOs

The designed antisense oligonucleotides (ASOs) targeting the 5′ and 3′ UTR region of a FOXG1 mRNA were tested for the ability to modulate (e.g. increase) FOXG1 expression in cells. In brief, cells were transfected with The ASOs of Table 1 ad Table 2, and the changes in FOXG1 mRNA were measured.

Cells:

HEK293 cells were obtained from ATCC (ATCC in partnership with LGC Standards, Wesel, Germany, cat. #ATCC-CRL-1573) and cultured in EMEM (#30-2003, ATCC in partnership with LGC Standards, Wesel, Germany), supplemented to contain 10% fetal calf serum (1248D, Biochrom GmbH, Berlin, Germany), and 100 U/ml Penicillin/100n/m1 Streptomycin (A2213, Biochrom GmbH, Berlin, Germany) at 37° C. in an atmosphere with 5% CO₂ in a humidified incubator. For transfection of HEK293 cells with ASOs, cells were seeded at a density of 15,000 cells/well into 96-well tissue culture plates (#655180, GBO, Germany).

Transfection of ASOs:

In HEK293 cells, transfection of ASOs was carried out with Lipofectamine2000 (Invitrogen/Life Technologies, Karlsruhe, Germany) according to manufacturer's instructions for reverse transfection with 0.5 μL Lipofectamine2000 per well.

The single dose screen was performed with ASOs in quadruplicates at 50 nM, with two ASOs targeting AHSA1 (one 2′-O-methoxyethyl (MOE) and one 2′-O-methyl (oMe) ASO) and a siRNA targeting RLuc as unspecific controls and a mock transfection. After 24h of incubation with ASOs, medium was removed and cells were lysed in 150111 Medium-Lysis Mixture (1 volume lysis mixture, 2 volumes cell culture medium) and then incubated at 53° C. for 30 minutes.

The two Ahsal-ASOs (one 2′-oMe-modified and one 2′-O-methoxyethyl (MOE MOE)-modified) served at the same time as unspecific controls for respective target mRNA expression and as a positive control to analyze transfection efficiency with regards to Ahsal mRNA level. By hybridization with an Ahsal probe set, the mock transfected wells served as controls for Ahsal mRNA level. Transfection efficiency for each 96-well plate and both doses in the dual dose screen were calculated by relating Ahsal-level with Ahsal-ASO (normalized to GapDH) to Ahsal-level obtained with mock controls.

Detection of FOXG1 mRNA:

QuantiGene detection was used to determine FOXG1 mRNA expression in cells lysates. In short, the QuantiGene assay directly measures target RNAs captured through probe hybridization and quantified through branched DNA technology that amplifies the signal. The signal is read using a Luminex or a luminometer for single targets. The assay measures RNA at the sample source, the assay avoids biases and variability inherent to extraction techniques and enzymatic manipulations. In addition, this direct measurement helps overcome issues with transcript degradation typically found in samples such as FFPE.

For the detection of FOXG1 mRNA, a Quantigene-Singleplex assay (1.0 for GapDH and 2.0 for FoxG1) was performed according to manufacturer's instructions (ThermoFisher, Germany). Luminescence was read using 1420 Luminescence Counter (WALLAC VICTOR Light, Perkin Elmer, Rodgau-Jtigesheim, Germany) following 30 minutes incubation at RT in the dark. The probe sets used for FOXG1 mRNA detection are set forth in Table 3 (Human FoxG1 QG2.0 probe set (Accession #NM_005249): Oligosequences “CEs” and “LEs” are depicted without the proprietary parts of their sequences. Cross reactivity with the cyno sequence was obtained by adding additional probes). Control GapDH probe sets are set forth in Table 5 (Human GapDH QG1.0 probe set (Accession #NM_002046): Oligosequences “CEs” and “LEs” are depicted without the proprietary parts of their sequences.).

TABLE 3
Human FoxG1 QG2.0 probe set (Accession #NM_005249)

| Oligo name | sequence 5′-3′ | accession#, position & function |
|---|---|---|
| QG2_hsFoxG1_1 | ggccagcttggcccg | NM 005249.1334.1348.LE |
| QG2_hsFoxG1 2 | gcgcaccgcgcttgaa | NM_005249.1349.1364.LE |
| QG2_hsFoxG1 3 | gccggtggaggtgaggc | NM_005249.1365.1381.CE |
| QG2_hsFoxG1_4 | cgcggtccatgaaggtgag | NM_005249.1382.1400.LE |
| QG2 hsFoxG1 5 | gccagtagagggagccgg | NM_005249.1401.1418.LE |
| QG2_hsFoxG1_6 | gacaggaagggcgacatgg | NM_005249.1419.1437.BL |
| QG2 hsFoxG1 7 | gcgggggtggtgcagg | NM_005249.1438.1453.BL |
| QG2_hsFoxG1_8 | tgtaactcaaagtgctgctggc | NM_005249.1454.1475.CE |
| QG2_hsFoxG1_9 | gccgacgtggtgccgt | NM_005249.1476.1491.LE |
| QG2_hsFoxG1_10 | atggggtggctggggtag | NM_005249.1492.1509.LE |
| QG2_hsFoxG1_11 | tcaacacggagctgtagggc | NM_005249.1510.1529.CE |
| QG2 hsFoxG1 12 | gttgcccagcgagttctgag | NM_005249.1530.1549.LE |
| QG2_hsFoxG1_13 | gcggtggagaaggagtggtt | NM_005249.1550.1569.LE |
| QG2 hsFoxG1 14 | ccacgctcaggccgttg | NM_005249.1570.1586.BL |
| QG2_hsFoxG1_15 | cccgttgaccagccggt | NM_005249.1587.1603.CE |
| QG2_hsFoxG1_16 | cgtggcgtacgggatctc | NM_005249.1604.1621.LE |
| QG2_hsFoxG1_17 | gcggccgtgaggtggtg | NM_005249.1622.1638.LE |
| QG2_hsFoxG1 18 | gaggcggctagcgcg | NM_005249.1639.1653.CE |
| QG2_hsFoxG1_19 | caggccgcagggcacc | NM_005249.1654.1669.LE |
| QG2_hsFoxG1 20 | ccagagcagggcaccga | NM_005249.1670.1686.LE |
| QG2_hsFoxG1 21 | caggggttgagggagtaggtc | NM_005249.1687.1707.CE |
| QG2_hsFoxG1 22 | gcgagcaggttgacggag | NM_005249.1708.1725.LE |
| QG2_hsFoxG1 23 | gaaaaagtaactggtctggccc | NM_005249.1726.1747.LE |
| QG2_hsFoxG1_24 | ggtgcgggacgtgggg | NM_005249.1748.1763.CE |
| QG2_hsFoxG1 25 | tgctctgcgaagtcattgacg | NM_005249.1764.1784.LE |
| QG2_hsFoxG1 26 | ggcgctcatggacgtgc | NM_005249.1785.1801.LE |
| QG2_hsFoxG1 27 | aggaggacgcggccct | NM_005249.1802.1817.CE |

TABLE 4
Human GapDH QG1.0 probe set (Accession #NM_002046)

| Oligo name | sequence 5′-3′ | accession#, position & function |
|---|---|---|
| QG1_hsGAP_1 | gaatttgccatgggtggaat | NM_002046.252.271.CE |
| QG1_hsGAP_2 | ggagggatctcgctcctgga | NM_002046.333.352.CE |
| QG1_hsGAP 3 | ccccagccttctccatggt | NM_002046.413.431.CE |
| QG1_hsGAP 4 | gctcccccctgcaaatgag | NM_002046.432.450.CE |
| QG1_hsGAP 5 | agccttgacggtgccatg | NM_002046.272.289.LE |
| QG1 hsGAP 6 | gatgacaagcttcccgttctc | NM_002046.290.310.LE |
| QG1_hsGAP 7 | agatggtgatgggatttccatt | NM_002046.311.332.LE |
| QG1_hsGAP_8 | gcatcgccccacttgatttt | NM_002046.353.372.LE |
| QG1_hsGAP_9 | cacgacgtactcagcgcca | NM_002046.373.391.LE |
| QG1_hsGAP_10 | ggcagagatgatgacccttttg | NM_002046.451.472.LE |
| QG1_hsGAP_11 | ggtgaagacgccagtggactc | NM_002046.392.412.BL |

Modulation of FOXG1 Expression by ASOs:

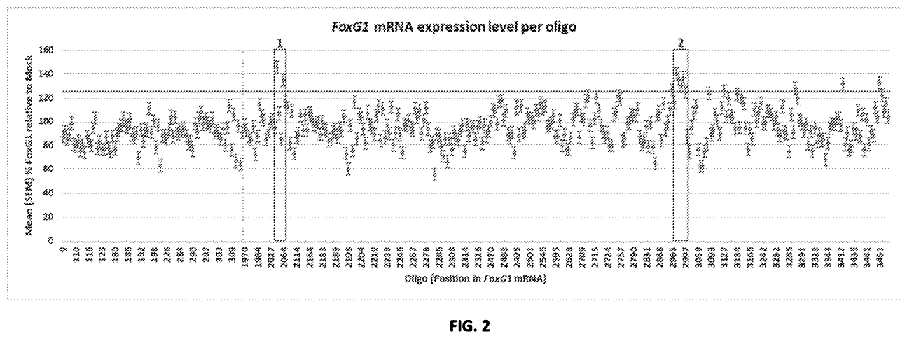

FIG. 2 shows FOXG1 mRNA expression data relative to mock transfection control. Each symbol (dot) indicates mean and standard error (bars). FoxG1 level as determined by linear model analysis. Oligos arranged in order of start position in FoxG1 mRNA (RefSeq NM_005249.5). Vertical dashed line indicates demarcation between 5′-UTR and 3′-UTR targeting oligos (left and right, respectively). The green line indicates 125% expression. Clusters 1 and 2, are indicated by purple boxes. The clusters are defined by 2 or more oligos sharing coordinate space and upregulating FoxG1>125%. For each well, the target mRNA level was normalized to the respective GAPDH mRNA level. Table 5 shows select sequences associated with the identified clusters. The activity of a given ASO was expressed as percent mRNA concentration of the respective target (normalized to GAPDH mRNA) in treated cells, relative to the target mRNA concentration (normalized to GAPDH mRNA) averaged across control wells (set as 100% target expression).

TABLE 5
ASO-mediated modulation of FOXG1 expression in cells

| | | | Mean % FoxG1 | |
|---|---|---|---|---|
| Oligo | Start | End | relative to Mock | Cluster |
| NM_005249.5_2061-2080_as | 2061 | 2080 | 145.58364 | 1 |
| (SEQ ID NO: 100) | | | | |
| NM_005249.5_2064-2083_as | 2064 | 2083 | 134.88537 | 1 |
| (SEQ ID NO: 103) | | | | |
| NM_005249.5_2965-2984_as | 2965 | 2984 | 126.46911 | 2 |
| (SEQ ID NO: 284) | | | | |
| NM_005249.5_2967-2986_as | 2967 | 2986 | 139.66475 | 2 |
| (SEQ ID NO: 286) | | | | |
| NM_005249.5_2968-2987_as | 2968 | 2987 | 135.56079 | 2 |
| (SEQ ID NO: 287) | | | | |
| NM_005249.5_2995-3014_as | 2995 | 3014 | 129.12053 | 2 |
| (SEQ ID NO: 288) | | | | |
| NM_005249.5_2996-3015_as | 2996 | 3015 | 136.41197 | 2 |
| (SEQ ID NO: 289) | | | | |

### Example 3: Cellular Modulation of FOXG1 Expression by Select ASOs in HEK293 Cells

The designed antisense oligonucleotides (ASOs) targeting a FOXG1 mRNA were further tested for the ability to modulate (e.g. increase) FOXG1 expression in cells. In brief, cells were transfected with The ASOs of Table 6, and the changes in FOXG1 mRNA were measured.

Transfection of ASOs and FOXG1 Quantification:

In HEK293 cells, transfection was performed with ASOs at concentrations of 50 nM and 10 nM in replicate. After 24h of incubation with ASOs, medium was removed, the cells were lysed, and QuantiGene detection was used to determine FOXG1 mRNA expression in cells lysates.

Modulation of FOXG1 Expression by ASOs:

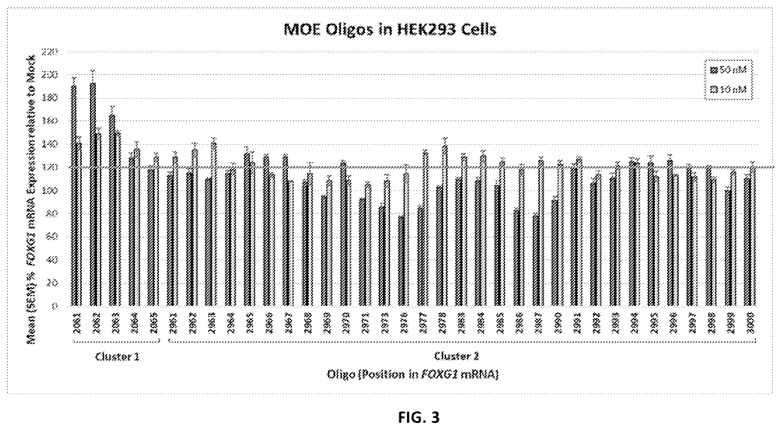

FIG. 3 shows FOXG1 mRNA expression modulation of selected 2′-O-methoxyethyl (MOE) chemistry oligos in HEK293, relative to mean of mock transfection control. Each bar indicates the mean and standard error FOXG1 level. ASOs are arranged by and listed in order of start position in FOXG1 mRNA (RefSeq NM_005249.5). The green horizontal line indicates 125% expression. Clusters 1 and 2 also noted. Table 6 shows the ASO coverage of the FOXG1 mRNA and data associated with the modulation of FOXG1 expression.

TABLE 6
ASO-mediated up-regulation of FOXG1 mRNA in cells

| Oligo (Position | | Mean | |
|---|---|---|---|
| in FOXG1 mRNA) | Dose | Expression | SEM |
| NM_005249.5_2061-2080 | 50 nM | 189.7648 | 7.739995 |
| NM_005249.5_2062-2081 | 50 nM | 192.3423 | 10.95742 |
| NM_005249.5_2063-2082 | 50 nM | 164.8299 | 7.865033 |
| NM_005249.5_2064-2083 | 50 nM | 127.9935 | 4.398258 |
| NM_005249.5_2065-2084 | 50 nM | 117.7618 | 3.856764 |
| NM_005249.5_2961-2980 | 50 nM | 112.9502 | 2.841189 |
| NM_005249.5_2962-2981 | 50 nM | 114.7827 | 4.184544 |
| NM_005249.5_2963-2982 | 50 nM | 109.707 | 0.913357 |
| NM_005249.5_2964-2983 | 50 nM | 114.5229 | 2.913248 |
| NM_005249.5_2965-2984 | 50 nM | 131.6638 | 5.676781 |
| NM_005249.5_2966-2985 | 50 nM | 129.4804 | 1.851186 |
| NM_005249.5_2967-2986 | 50 nM | 128.9098 | 2.447689 |
| NM_005249.5_2968-2987 | 50 nM | 107.1351 | 1.832585 |
| NM_005249.5_2969-2988 | 50 nM | 94.31892 | 1.188665 |
| NM_005249.5_2970-2989 | 50 nM | 123.675 | 1.774876 |
| NM_005249.5_2971-2990 | 50 nM | 92.11175 | 1.043745 |
| NM_005249.5_2973-2992 | 50 nM | 85.85752 | 3.003942 |
| NM_005249.5_2976-2995 | 50 nM | 76.77638 | 1.550449 |
| NM_005249.5_2977-2996 | 50 nM | 84.87921 | 1.6896 |
| NM_005249.5_2978-2997 | 50 nM | 102.624 | 1.407233 |
| NM_005249.5_2983-3002 | 50 nM | 109.6413 | 1.645209 |
| NM_005249.5_2984-3003 | 50 nM | 108.0409 | 2.905723 |
| NM_005249.5_2985-3004 | 50 nM | 104.6014 | 3.465679 |
| NM_005249.5_2986-3005 | 50 nM | 83.09921 | 1.444432 |
| NM_005249.5_2987-3006 | 50 nM | 77.87864 | 2.458964 |
| NM_005249.5_2990-3009 | 50 nM | 91.60617 | 3.409702 |
| NM_005249.5_2991-3010 | 50 nM | 119.3121 | 3.504208 |
| NM_005249.5_2992-3011 | 50 nM | 106.3858 | 4.279597 |
| NM_005249.5_2993-3012 | 50 nM | 110.7718 | 4.264335 |
| NM_005249.5_2994-3013 | 50 nM | 125.111 | 3.311955 |
| NM_005249.5_2995-3014 | 50 nM | 123.881 | 5.910818 |
| NM_005249.5_2996-3015 | 50 nM | 125.3415 | 5.550329 |
| NM_005249.5_2997-3016 | 50 nM | 119.9982 | 2.415439 |
| NM_005249.5_2998-3017 | 50 nM | 119.8153 | 2.011818 |
| NM_005249.5_2999-3018 | 50 nM | 100.3009 | 2.463369 |
| NM_005249.5_3000-3019 | 50 nM | 110.0815 | 3.525977 |
| NM_005249.5_2061-2080 | 10 nM | 140.8695 | 5.409641 |
| NM_005249.5_2062-2081 | 10 nM | 148.9523 | 4.47351 |
| NM_005249.5_2063-2082 | 10 nM | 149.4905 | 2.028402 |
| NM_005249.5_2064-2083 | 10 nM | 135.3995 | 6.766115 |
| NM_005249.5_2065-2084 | 10 nM | 128.6393 | 3.486294 |
| NM_005249.5_2961-2980 | 10 nM | 128.9611 | 4.7843 |
| NM_005249.5_2962-2981 | 10 nM | 134.9864 | 5.806415 |
| NM_005249.5_2963-2982 | 10 nM | 140.5912 | 4.537928 |
| NM_005249.5_2964-2983 | 10 nM | 118.3183 | 5.061172 |
| NM_005249.5_2965-2984 | 10 nM | 124.083 | 9.098639 |
| NM_005249.5_2966-2985 | 10 nM | 113.5794 | 1.977667 |
| NM_005249.5_2967-2986 | 10 nM | 108.0511 | 0.430458 |
| NM_005249.5_2968-2987 | 10 nM | 114.3724 | 9.577348 |
| NM_005249.5_2969-2988 | 10 nM | 108.5649 | 3.977983 |
| NM_005249.5_2970-2989 | 10 nM | 108.5442 | 3.768629 |
| NM_005249.5_2971-2990 | 10 nM | 104.7672 | 2.365784 |
| NM_005249.5_2973-2992 | 10 nM | 108.0177 | 5.491231 |
| NM_005249.5_2976-2995 | 10 nM | 114.5418 | 7.586278 |
| NM_005249.5_2977-2996 | 10 nM | 132.8276 | 2.279475 |
| NM_005249.5_2978-2997 | 10 nM | 138.4885 | 6.397771 |
| NM_005249.5_2983-3002 | 10 nM | 128.7813 | 2.926409 |
| NM_005249.5_2984-3003 | 10 nM | 129.6681 | 4.946237 |
| NM_005249.5_2985-3004 | 10 nM | 124.5868 | 3.105648 |
| NM_005249.5_2986-3005 | 10 nM | 118.2728 | 4.379385 |
| NM_005249.5_2987-3006 | 10 nM | 125.4329 | 3.341276 |
| NM_005249.5_2990-3009 | 10 nM | 122.72 | 3.189793 |
| NM_005249.5_2991-3010 | 10 nM | 126.7657 | 2.150985 |
| NM_005249.5_2992-3011 | 10 nM | 113.4971 | 3.562776 |
| NM_005249.5_2993-3012 | 10 nM | 121.0352 | 3.209476 |
| NM_005249.5_2994-3013 | 10 nM | 123.4705 | 3.868376 |
| NM_005249.5_2995-3014 | 10 nM | 112.2469 | 4.423879 |
| NM_005249.5_2996-3015 | 10 nM | 113.204 | 0.847541 |
| NM_005249.5_2997-3016 | 10 nM | 111.7264 | 3.5779 |
| NM_005249.5_2998-3017 | 10 nM | 108.964 | 2.369043 |
| NM_005249.5_2999-3018 | 10 nM | 115.8594 | 2.530501 |
| NM_005249.5_3000-3019 | 10 nM | 119.797 | 4.63932 |

### Example 4: Cellular Modulation of FOXG1 Expression by Select ASOs in CFF-STTG1 and SW1783 Cells

The designed antisense oligonucleotides (ASOs) targeting a FOXG1 mRNA were tested for the ability to modulate (e.g. increase) FOXG1 expression in brain tissue-derived cells. In brief, cells were transfected with to ASOs of Table 7, and the changes in FOXG1 mRNA were measured.

Transfection of ASOs and FOXG1 Quantification:

In CFF-STTG1 and SW1783 cells, transfection was performed with ASOs at concentrations of 50 nM and 10 nM, in replicate. After 24h of incubation with ASOs, medium was removed, the cells were lysed, and QuantiGene detection was used to determine FOXG1 mRNA expression in cells lysates.

Modulation of FOXG1 Expression by ASOs:

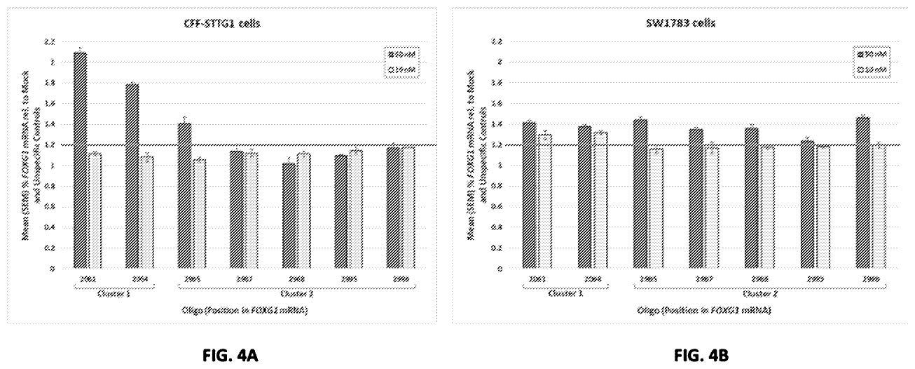

FIG. 4A shows FOXG1 mRNA expression modulation of selected 2′-O-methoxyethyl (MOE) chemistry oligos in CFF-STTG1 cells, relative to mean of mock transfection and nonspecific oligo controls. FIG. 4B shows FOXG1 mRNA expression modulation of selected oligos in SW1783 cells, relative to mean of mock transfection and nonspecific oligo controls. For both FIG. 4A and FIG. 4B, each bar indicates mean and standard error FOXG1 level and ASOs are arranged by and listed in order of start position in FOXG1 mRNA (RefSeq NM_005249.5). The green horizontal line indicates 125% expression and clusters 1-2 are noted. Table 7 shows ASO coverage of the FOXG1 mRNA and data associated with the modulation of FOXG1 expression in CFF-STTG1 and SW1783 cell lines.

TABLE 7
ASO-mediated upregulation of FOXG1 mRNA in CFF-STTG1 and SW1783 cells

| Oligo | | | Mean | |
|---|---|---|---|---|
| (Position in FoxG1 mRNA) | Cell Line | Dose | Expression | SEM |
| NM_005249.5_2061-2080_as | CFF-STTG1 | 50 nM | 2.09060354 | 0.0524632 |
| NM_005249.5_2064-2083_as | CFF-STTG1 | 50 nM | 1.78106746 | 0.02497863 |
| NM_005249.5_2965-2984_as | CFF-STTG1 | 50 nM | 1.40656881 | 0.06326815 |
| NM_005249.5_2967-2986_as | CFF-STTG1 | 50 nM | 1.14106306 | 0.06401273 |
| NM_005249.5_2968-2987_as | CFF-STTG1 | 50 nM | 1.01822144 | 0.05812383 |
| NM_005249.5_2995-3014_as | CFF-STTG1 | 50 nM | 1.0966339 | 0.00706128 |
| NM_005249.5_2996-3015_as | CFF-STTG1 | 50 nM | 1.17138666 | 0.04592333 |
| NM_005249.5_2061-2080_as | CFF-STTG1 | 10 nM | 1.11463161 | 0.01828397 |
| NM_005249.5_2064-2083_as | CFF-STTG1 | 10 nM | 1.08309632 | 0.04509828 |
| NM_005249.5_2965-2984_as | CFF-STTG1 | 10 nM | 1.05531127 | 0.02590015 |
| NM_005249.5_2967-2986_as | CFF-STTG1 | 10 nM | 1.11894287 | 0.03515521 |
| NM_005249.5_2968-2987_as | CFF-STTG1 | 10 nM | 1.11193636 | 0.02863519 |
| NM_005249.5_2995-3014_as | CFF-STTG1 | 10 nM | 1.14476513 | 0.0331245 |
| NM_005249.5_2996-3015_as | CFF-STTG1 | 10 nM | 1.17782235 | 0.00312998 |
| NM_005249.5_2061-2080_as | SW1783 | 50 nM | 1.41432605 | 0.02330619 |
| NM_005249.5_2064-2083_as | SW1783 | 50 nM | 1.37415916 | 0.01947226 |
| NM_005249.5_2965-2984_as | SW1783 | 50 nM | 1.43663656 | 0.03060538 |
| NM_005249.5_2967-2986_as | SW1783 | 50 nM | 1.34452967 | 0.02806401 |
| NM_005249.5_2968-2987_as | SW1783 | 50 nM | 1.35678534 | 0.0400883 |
| NM_005249.5_2995-3014_as | SW1783 | 50 nM | 1.23298541 | 0.04153227 |
| NM_005249.5_2996-3015_as | SW1783 | 50 nM | 1.46154338 | 0.02879713 |
| NM_005249.5_2061-2080_as | SW1783 | 10 nM | 1.29423388 | 0.04532559 |
| NM_005249.5_2064-2083_as | SW1783 | 10 nM | 1.31686659 | 0.01826147 |
| NM_005249.5_2965-2984_as | SW1783 | 10 nM | 1.15913468 | 0.04184637 |
| NM_005249.5_2967-2986_as | SW1783 | 10 nM | 1.17039018 | 0.05614856 |
| NM_005249.5_2968-2987_as | SW1783 | 10 nM | 1.17738434 | 0.01821765 |
| NM_005249.5_2995-3014_as | SW1783 | 10 nM | 1.18240062 | 0.01173471 |
| NM_005249.5_2996-3015_as | SW1783 | 10 nM | 1.195674 | 0.02501848 |

While preferred embodiments of the present disclosure have been shown and described herein, it will be obvious to those skilled in the art that such embodiments are provided by way of example only. Numerous variations, changes, and substitutions will now occur to those skilled in the art without departing from the present disclosure. It should be understood that various alternatives to the embodiments of the present disclosure described herein may be employed in practicing the present disclosure. It is intended that the following claims define the scope of the present disclosure and that methods and structures within the scope of these claims and their equivalents be covered thereby.

SEQUENCES

| SEQ ID NO | SEQUENCE |
|---|---|
| 1 | AGCGATCGAGGCGGCTATAG |
| 2 | CAGCGATCGAGGCGGCTATA |
| 3 | ACAGCGATCGAGGCGGCTAT |
| 4 | GACAGCGATCGAGGCGGCTA |
| 5 | AGACAGCGATCGAGGCGGCT |
| 6 | GCAGCAGTCACAGCAGCAGC |
| 7 | CGCAGCAGCAGTCACAGCAG |
| 8 | TCGCAGCAGCAGTCACAGCA |
| 9 | CTCGCAGCAGCAGTCACAGC |
| 10 | TCTCGCAGCAGCAGTCACAG |
| 11 | CTCTCGCAGCAGCAGTCACA |
| 12 | CCTCTCGCAGCAGCAGTCAC |
| 13 | TCCTCTCGCAGCAGCAGTCA |
| 14 | CTCCTCTCGCAGCAGCAGTC |
| 15 | CCTCCTCTCGCAGCAGCAGT |
| 16 | TCCTCCTCTCGCAGCAGCAG |
| 17 | CTCCTCCTCTCGCAGCAGCA |
| 18 | TCCTCCTCCTCTCGCAGCAG |
| 19 | CTCCTCCTCCTCTCGCAGCA |
| 20 | TCCTCCTCCTCCTCTCGCAG |
| 21 | CTCCTCCTCCTCCTCTCGCA |
| 22 | GCTGCTTCCTCCTCCTCCTC |
| 23 | CGCTGCTTCCTCCTCCTCCT |
| 24 | TGTACTTCTTGGTCTCCCCC |
| 25 | CTGTACTTCTTGGTCTCCCC |
| 26 | ACTGTACTTCTTGGTCTCCC |
| 27 | AACTGTACTTCTTGGTCTCC |
| 28 | CAACTGTACTTCTTGGTCTC |
| 29 | CCAACTGTACTTCTTGGTCT |
| 30 | CCCAACTGTACTTCTTGGTC |
| 31 | TCCCAACTGTACTTCTTGGT |
| 32 | CTCCCAACTGTACTTCTTGG |
| 33 | GCTCCCAACTGTACTTCTTG |
| 34 | CGCTCCCAACTGTACTTCTT |
| 35 | TCGCTCCCAACTGTACTTCT |
| 36 | CTCGCTCCCAACTGTACTTC |
| 37 | CCTCGCTCCCAACTGTACTT |
| 38 | CCCTCGCTCCCAACTGTACT |
| 39 | TCCCTCGCTCCCAACTGTAC |
| 40 | CTCCCTCGCTCCCAACTGTA |
| 41 | GCTCCCTCGCTCCCAACTGT |
| 42 | AGCTCCCTCGCTCCCAACTG |
| 43 | AAGCTCCCTCGCTCCCAACT |
| 44 | GAAGCTCCCTCGCTCCCAAC |
| 45 | TGAAGCTCCCTCGCTCCCAA |
| 46 | GTGAAGCTCCCTCGCTCCCA |
| 47 | AAGAAACAACCACCGCCCCG |
| 48 | AAAGAAACAACCACCGCCCC |
| 49 | AAAAGAAACAACCACCGCCC |
| 50 | AAAAAGAAACAACCACCGCC |
| 51 | CCCCTCAGGAATTAGAAAAA |
| 52 | ACCCCTCAGGAATTAGAAAA |
| 53 | CACCCCTCAGGAATTAGAAA |
| 54 | CCACCCCTCAGGAATTAGAA |
| 55 | ACCACCCCTCAGGAATTAGA |
| 56 | AACCACCCCTCAGGAATTAG |
| 57 | CAACCACCCCTCAGGAATTA |
| 58 | GCAACCACCCCTCAGGAATT |
| 59 | AGCAACCACCCCTCAGGAAT |
| 60 | CAGCAACCACCCCTCAGGAA |
| 61 | GCAGCAACCACCCCTCAGGA |
| 62 | AAGCAGCAACCACCCCTCAG |
| 63 | AAAGCAGCAACCACCCCTCA |
| 64 | AAAAGCAGCAACCACCCCTC |
| 65 | CAAAAGCAGCAACCACCCCT |
| 66 | GCAAAAGCAGCAACCACCCC |
| 67 | AGCAAAAGCAGCAACCACCC |
| 68 | TAGCAAAAGCAGCAACCACC |
| 69 | GTAGCAAAAGCAGCAACCAC |
| 70 | TGTAGCAAAAGCAGCAACCA |
| 71 | ATGTAGCAAAAGCAGCAACC |
| 72 | CATGTAGCAAAAGCAGCAAC |
| 73 | TCATGTAGCAAAAGCAGCAA |
| 74 | GTCATGTAGCAAAAGCAGCA |
| 75 | AGTCATGTAGCAAAAGCAGC |
| 76 | AAGTCATGTAGCAAAAGCAG |
| 77 | CAAGTCATGTAGCAAAAGCA |
| 78 | GCAAGTCATGTAGCAAAAGC |
| 79 | GGCAAGTCATGTAGCAAAAG |
| 80 | TGGCAAGTCATGTAGCAAAA |
| 81 | CTGGCAAGTCATGTAGCAAA |
| 82 | GCTGGCAAGTCATGTAGCAA |
| 83 | CGCTGGCAAGTCATGTAGCA |
| 84 | GCGCTGGCAAGTCATGTAGC |
| 85 | TCACTTACAGTCTGGTCCCA |
| 86 | TTCACTTACAGTCTGGTCCC |
| 87 | ACGTTCACTTACAGTCTGGT |
| 88 | GTGTAAAACGTTCACTTACA |
| 89 | TGTGTAAAACGTTCACTTAC |
| 90 | GTGTGTAAAACGTTCACTTA |
| 91 | TGTGTGTAAAACGTTCACTT |
| 92 | TGCAAATGTGTGTAAAACGT |
| 93 | ATGCAAATGTGTGTAAAACG |
| 94 | AATGCAAATGTGTGTAAAAC |
| 95 | CAATGCAAATGTGTGTAAAA |
| 96 | TTTACAATGCAAATGTGTGT |
| 97 | AAATACCTGGACTTATTTTT |
| 98 | AAAATACCTGGACTTATTTT |
| 99 | AAAAATACCTGGACTTATTT |
| 100 | AACGTACAGAAATGGGAGGG |
| 101 | AAACGTACAGAAATGGGAGG |
| 102 | CAAACGTACAGAAATGGGAG |
| 103 | ACAAACGTACAGAAATGGGA |
| 104 | AACAAACGTACAGAAATGGG |
| 105 | GAACAAACGTACAGAAATGG |
| 106 | CACTCCACACCTTGTTAGAA |
| 107 | ACACTCCACACCTTGTTAGA |
| 108 | GACACTCCACACCTTGTTAG |
| 109 | TCGCTGACACTCCACACCTT |
| 110 | GTATTCTCCCCACATTGCAC |
| 111 | TGTATTCTCCCCACATTGCA |
| 112 | ATGTATTCTCCCCACATTGC |
| 113 | ACAATGTATTCTCCCCACAT |
| 114 | TTGACTTCCAAACCTTATAT |
| 115 | TTTGACTTCCAAACCTTATA |
| 116 | CTACTATAATTTGACTTCCA |
| 117 | TCTACTATAATTTGACTTCC |
| 118 | TTCTACTATAATTTGACTTC |
| 119 | CATTCTACTATAATTTGACT |
| 120 | ACATTCTACTATAATTTGAC |
| 121 | GATACACATTCTACTATAAT |
| 122 | AGATACACATTCTACTATAA |
| 123 | TAGATACACATTCTACTATA |
| 124 | TTAGATACACATTCTACTAT |
| 125 | TTTAGATACACATTCTACTA |
| 126 | ATTTAGATACACATTCTACT |
| 127 | TATTTAGATACACATTCTAC |
| 128 | CTATTTAGATACACATTCTA |
| 129 | CACTATTTAGATACACATTC |
| 130 | GTCACTATTTAGATACACAT |
| 131 | AGTCACTATTTAGATACACA |
| 132 | CAGTCACTATTTAGATACAC |
| 133 | AGCAGTCACTATTTAGATAC |
| 134 | AAGCAGTCACTATTTAGATA |
| 135 | AAAGCAGTCACTATTTAGAT |
| 136 | CAAAGCAGTCACTATTTAGA |
| 137 | GCAAAGCAGTCACTATTTAG |
| 138 | GGCAAAGCAGTCACTATTTA |
| 139 | TGGCAAAGCAGTCACTATTT |
| 140 | AATGGCAAAGCAGTCACTAT |
| 141 | AAATGGCAAAGCAGTCACTA |
| 142 | GAAATGGCAAAGCAGTCACT |
| 143 | AATGAAATGGCAAAGCAGTC |
| 144 | AGGTTTGAATGAAATGGCAA |
| 145 | CAGGTTTGAATGAAATGGCA |
| 146 | TCAGGTTTGAATGAAATGGC |
| 147 | GTCAGGTTTGAATGAAATGG |
| 148 | CTTGTCAGGTTTGAATGAAA |
| 149 | CTTAGAGATAGACTTGTCAG |
| 150 | TCTTAGAGATAGACTTGTCA |
| 151 | CTCTTAGAGATAGACTTGTC |
| 152 | GCTCTTAGAGATAGACTTGT |
| 153 | GGCTCTTAGAGATAGACTTG |
| 154 | CGGCTCTTAGAGATAGACTT |
| 155 | GCGGCTCTTAGAGATAGACT |
| 156 | TGGCGGCTCTTAGAGATAGA |
| 157 | TCTGGCGGCTCTTAGAGATA |
| 158 | ATCTGGCGGCTCTTAGAGAT |
| 159 | AATCTGGCGGCTCTTAGAGA |
| 160 | TACTGCACACATGGAAATCT |
| 161 | ATACTGCACACATGGAAATC |
| 162 | AATACTGCACACATGGAAAT |
| 163 | ATAATACTGCACACATGGAA |
| 164 | CTTATAATACTGCACACATG |
| 165 | AACTTATAATACTGCACACA |
| 166 | TAACTTATAATACTGCACAC |
| 167 | ATAACTTATAATACTGCACA |
| 168 | GATAACTTATAATACTGCAC |
| 169 | TGATAACTTATAATACTGCA |
| 170 | ATGATAACTTATAATACTGC |
| 171 | GTTCCATGATAACTTATAAT |
| 172 | AGTTCCATGATAACTTATAA |
| 173 | TAGTTCCATGATAACTTATA |
| 174 | ATAGTTCCATGATAACTTAT |
| 175 | TATAGTTCCATGATAACTTA |
| 176 | TCTGCGTCCACCATATAGTT |
| 177 | GTCTGCGTCCACCATATAGT |
| 178 | GGTCTGCGTCCACCATATAG |
| 179 | AGGTCTGCGTCCACCATATA |
| 180 | AAGGTCTGCGTCCACCATAT |
| 181 | TTCTCAAGGTCTGCGTCCAC |
| 182 | GTTCTCAAGGTCTGCGTCCA |
| 183 | TGTTCTCAAGGTCTGCGTCC |
| 184 | TTGTTCTCAAGGTCTGCGTC |
| 185 | GTTGTTCTCAAGGTCTGCGT |
| 186 | GGTTGTTCTCAAGGTCTGCG |
| 187 | AGGTTGTTCTCAAGGTCTGC |
| 188 | TAGGTTGTTCTCAAGGTCTG |
| 189 | TTAGGTTGTTCTCAAGGTCT |
| 190 | TTTAGGTTGTTCTCAAGGTC |
| 191 | AATTTAGGTTGTTCTCAAGG |
| 192 | CCCATAATTTAGGTTGTTCT |
| 193 | CCCCATAATTTAGGTTGTTC |
| 194 | TCCCCATAATTTAGGTTGTT |
| 195 | CTCCCCATAATTTAGGTTGT |
| 196 | TCTCCCCATAATTTAGGTTG |
| 197 | AAATTCTCCCCATAATTTAG |
| 198 | CAATAAATGGCCAAAATAAT |
| 199 | TCTTTGGTCTAAAAGTAAAC |
| 200 | ATCTTTGGTCTAAAAGTAAA |
| 201 | AATCTTTGGTCTAAAAGTAA |
| 202 | CAATCTTTGGTCTAAAAGTA |
| 203 | TTTCTAGAACCCAATCTTTG |
| 204 | CATTTTCTAGAACCCAATCT |
| 205 | GCATTTTCTAGAACCCAATC |
| 206 | TGCATTTTCTAGAACCCAAT |
| 207 | GTGCATTTTCTAGAACCCAA |
| 208 | AGTGCATTTTCTAGAACCCA |
| 209 | CAAGTGCATTTTCTAGAACC |
| 210 | CCAAGTGCATTTTCTAGAAC |
| 211 | ACCAAGTGCATTTTCTAGAA |
| 212 | TACCAAGTGCATTTTCTAGA |
| 213 | ATACCAAGTGCATTTTCTAG |
| 214 | TATACCAAGTGCATTTTCTA |
| 215 | GTATACCAAGTGCATTTTCT |
| 216 | AGTATACCAAGTGCATTTTC |
| 217 | TAGTATACCAAGTGCATTTT |
| 218 | TTAGTATACCAAGTGCATTT |
| 219 | ACTTAGTATACCAAGTGCAT |
| 220 | TACTTAGTATACCAAGTGCA |
| 221 | ATACTTAGTATACCAAGTGC |
| 222 | AATACTTAGTATACCAAGTG |
| 223 | GTTTTAATACTTAGTATACC |
| 224 | AGTGTTGCCAACTGAAACAA |
| 225 | CAATTGAATGGGCAGTGTTG |
| 226 | TCAATTGAATGGGCAGTGTT |
| 227 | TTCAATTGAATGGGCAGTGT |
| 228 | TGAAGGCAATCGTTAATTTT |
| 229 | CTGAAGGCAATCGTTAATTT |
| 230 | ACTGAAGGCAATCGTTAATT |
| 231 | AACTGAAGGCAATCGTTAAT |
| 232 | AAACTGAAGGCAATCGTTAA |
| 233 | CAAACTGAAGGCAATCGTTA |
| 234 | ACAAACTGAAGGCAATCGTT |
| 235 | ACACAAACTGAAGGCAATCG |
| 236 | GTGACCACATACATCAAAAT |
| 237 | TTAGTGACCACATACATCAA |
| 238 | TTTACCTATAAGTACAATAG |
| 239 | GTTTACCTATAAGTACAATA |
| 240 | GGTTTACCTATAAGTACAAT |
| 241 | ACATATTTGCAAGGTTTACC |
| 242 | TACATATTTGCAAGGTTTAC |
| 243 | TTACATATTTGCAAGGTTTA |
| 244 | GTTACATATTTGCAAGGTTT |
| 245 | GGTTACATATTTGCAAGGTT |
| 246 | AGGTTACATATTTGCAAGGT |
| 247 | CAGGTTACATATTTGCAAGG |
| 248 | ACAGGTTACATATTTGCAAG |
| 249 | ACACAGGTTACATATTTGCA |
| 250 | AACACAGGTTACATATTTGC |
| 251 | GCAACACAGGTTACATATTT |
| 252 | GCGCAACACAGGTTACATAT |
| 253 | TGCGCAACACAGGTTACATA |
| 254 | TTGCGCAACACAGGTTACAT |
| 255 | TTTGCGCAACACAGGTTACA |
| 256 | CATTTGCGCAACACAGGTTA |
| 257 | ACTCAAATTTATGCGGCATT |
| 258 | ATCACTCAAATTTATGCGGC |
| 259 | ACATTAACAATCACTCAAAT |
| 260 | CAACATTAACAATCACTCAA |
| 261 | ACAACATTAACAATCACTCA |
| 262 | GACAACATTAACAATCACTC |
| 263 | AGACAACATTAACAATCACT |
| 264 | ACCACAGTATCACAATCAAG |
| 265 | GACCACAGTATCACAATCAA |
| 266 | TGACCACAGTATCACAATCA |
| 267 | ATGACCACAGTATCACAATC |
| 268 | CATATGACCACAGTATCACA |
| 269 | GCATATGACCACAGTATCAC |
| 270 | GACAAACACGGGCATATGAC |
| 271 | TGACAAACACGGGCATATGA |
| 272 | GTTCATAGTAAACATTTTTG |
| 273 | GTGTTCATAGTAAACATTTT |
| 274 | TGTGTTCATAGTAAACATTT |
| 275 | TCTGTGTGTTCATAGTAAAC |
| 276 | TTCTGTGTGTTCATAGTAAA |
| 277 | TATTTCTGTGTGTTCATAGT |
| 278 | GATATATATGAATTTAGCCT |
| 279 | AGATATATATGAATTTAGCC |
| 280 | AGACAAAAGTATCAAGATAT |
| 281 | AGTTGATTGGTCTTTAAAAA |
| 282 | CCCTATAAGTTGATTGGTCT |
| 283 | AAAAAGCCTTTGAATTCCCT |
| 284 | TAAATTTTAGTTTGGCTGAA |
| 285 | TTAAATTTTAGTTTGGCTGA |
| 286 | TTTAAATTTTAGTTTGGCTG |
| 287 | GTTTAAATTTTAGTTTGGCT |
| 288 | TTAGAGTCAGTTCAAATTAA |
| 289 | TTTAGAGTCAGTTCAAATTA |
| 290 | TTTTAGAGTCAGTTCAAATT |
| 291 | TCATTTTTAGAGTCAGTTCA |
| 292 | TTCATTTTTAGAGTCAGTTC |
| 293 | GTTCACAAAGGGAAAAATAC |
| 294 | CTGCTCCTTGTAAAATTTGT |
| 295 | GCTGCTCCTTGTAAAATTTG |
| 296 | TGTTTATTAAATAGGCTGCT |
| 297 | GTGTTTATTAAATAGGCTGC |
| 298 | TAGTGTTTATTAAATAGGCT |
| 299 | CTAGTGTTTATTAAATAGGC |
| 300 | GCTAGTGTTTATTAAATAGG |
| 301 | AAAGCCTATACTTTGTTTAA |
| 302 | TCAGCTGAAAAGCCTATACT |
| 303 | ATCAGCTGAAAAGCCTATAC |
| 304 | TATCAGCTGAAAAGCCTATA |
| 305 | GTATCAGCTGAAAAGCCTAT |
| 306 | GGTATCAGCTGAAAAGCCTA |
| 307 | TGTATATCCACAGAAACTTA |
| 308 | CTTTTTGCTGTATATCCACA |
| 309 | TCTTTTTGCTGTATATCCAC |
| 310 | CTCTTTTTGCTGTATATCCA |
| 311 | TCTCTTTTTGCTGTATATCC |
| 312 | ATCTCTTTTTGCTGTATATC |
| 313 | ATATCTCTTTTTGCTGTATA |
| 314 | TATATCTCTTTTTGCTGTAT |
| 315 | TTATATCTCTTTTTGCTGTA |
| 316 | ATTATATCTCTTTTTGCTGT |
| 317 | AATTATATCTCTTTTTGCTG |
| 318 | GGTAAAGAGCTATGCACAGA |
| 319 | GGGTAAAGAGCTATGCACAG |
| 320 | AGGGTAAAGAGCTATGCACA |
| 321 | CAGGGTAAAGAGCTATGCAC |
| 322 | ACAGGGTAAAGAGCTATGCA |
| 323 | AACACAGGGTAAAGAGCTAT |
| 324 | GCCAAGCTCTATTAACAATA |
| 325 | TGCCAAGCTCTATTAACAAT |
| 326 | TTGCCAAGCTCTATTAACAA |
| 327 | TTTGCCAAGCTCTATTAACA |
| 328 | ATAATTTGCCAAGCTCTATT |
| 329 | TATAATTTGCCAAGCTCTAT |
| 330 | TTATAATTTGCCAAGCTCTA |
| 331 | ATTTATAATTTGCCAAGCTC |
| 332 | TATTTATAATTTGCCAAGCT |
| 333 | TTATTTATAATTTGCCAAGC |
| 334 | ACTTCTATCTAACCATATAC |
| 335 | GTCACTTCTATCTAACCATA |
| 336 | AGTCACTTCTATCTAACCAT |
| 337 | TAGTCACTTCTATCTAACCA |
| 338 | ATAGTCACTTCTATCTAACC |
| 339 | TATAGTCACTTCTATCTAAC |
| 340 | TTATAGTCACTTCTATCTAA |
| 341 | ATTATAGTCACTTCTATCTA |
| 342 | CATTATAGTCACTTCTATCT |
| 343 | GCATTATAGTCACTTCTATC |
| 344 | TGCATTATAGTCACTTCTAT |
| 345 | GTGCATTATAGTCACTTCTA |
| 346 | GGGCTCTGTGTGTCTATATA |
| 347 | AGGGCTCTGTGTGTCTATAT |
| 348 | AAGGGCTCTGTGTGTCTATA |
| 349 | GAAGGGCTCTGTGTGTCTAT |
| 350 | TGAAGGGCTCTGTGTGTCTA |
| 351 | ACTGAAGGGCTCTGTGTGTC |
| 352 | GAACTGAAGGGCTCTGTGTG |
| 353 | TGAACTGAAGGGCTCTGTGT |
| 354 | CTGAACTGAAGGGCTCTGTG |
| 355 | CCTGAACTGAAGGGCTCTGT |
| 356 | AAATTGTACCTGAACTGAAG |
| 357 | CAAATTGTACCTGAACTGAA |
| 358 | GCAAATTGTACCTGAACTGA |
| 359 | CGCAAATTGTACCTGAACTG |
| 360 | GCGCAAATTGTACCTGAACT |
| 361 | ATAAATGCTGACTTAGAAAG |
| 362 | AAATAAATGCTGACTTAGAA |
| 363 | AAAATAAATGCTGACTTAGA |
| 364 | GTGGGTAAACAGCCACAAAA |
| 365 | TGTGGGTAAACAGCCACAAA |
| 366 | ATTGTGGGTAAACAGCCACA |
| 367 | CATTGTGGGTAAACAGCCAC |
| 368 | TCATTGTGGGTAAACAGCCA |
| 369 | TTCATTGTGGGTAAACAGCC |
| 370 | TTTCATTGTGGGTAAACAGC |
| 371 | CTTTCATTGTGGGTAAACAG |
| 372 | TCTTTCATTGTGGGTAAACA |
| 373 | CTCTTTCATTGTGGGTAAAC |
| 374 | ACTCTTTCATTGTGGGTAAA |
| 375 | AACTCTTTCATTGTGGGTAA |
| 376 | GAACTCTTTCATTGTGGGTA |
| 377 | AGAACTCTTTCATTGTGGGT |
| 378 | TAGAACTCTTTCATTGTGGG |
| 379 | TTAGAACTCTTTCATTGTGG |
| 380 | CTTTATTAGAACTCTTTCAT |
| 381 | ACATCTTTATTAGAACTCTT |
| 382 | GCACATCTTTATTAGAACTC |
| 383 | CAGCACATCTTTATTAGAAC |
| 384 | TCAGCACATCTTTATTAGAA |

1-82. (canceled)

83. An antisense oligonucleotide comprising an antisense oligonucleotide sequence that hybridizes to a target nucleic acid sequence located within positions 2000-2100 or 2900-3000 of a FOXG1 nucleic acid.

84. The antisense oligonucleotide of claim 83, wherein the antisense oligonucleotide sequence comprises SEQ ID NO: 100, SEQ ID NO:103, SEQ ID NO: 284, SEQ ID NO: 286, SEQ ID NO: 287, SEQ ID NO: 288, or SEQ ID NO: 289.

85. The antisense oligonucleotide of claim 83, wherein the antisense oligonucleotide hybridizes to one or more nucleotides within or adjacent to a position on the FOXG1 nucleic acid targeted by SEQ ID NO: 100, SEQ ID NO:103, SEQ ID NO: 284, SEQ ID NO: 286, SEQ ID NO: 287, SEQ ID NO: 288, or SEQ ID NO: 289.

86. The antisense oligonucleotide of claim 83, wherein the antisense oligonucleotide sequence comprises 90% sequence identity or greater to SEQ ID NO: 100, SEQ ID NO: 103, SEQ ID NO: 284, SEQ ID NO: 286, SEQ ID NO: 287, SEQ ID NO: 288, or SEQ ID NO: 289.

87. The antisense oligonucleotide of claim 83, wherein the antisense oligonucleotide sequence comprises 10 or more contiguous nucleotides selected from a sequence within SEQ ID NO: 100, SEQ ID NO: 103, SEQ ID NO: 284, SEQ ID NO: 286, SEQ ID NO: 287, SEQ ID NO: 288, or SEQ ID NO: 289.

88. The antisense oligonucleotide of claim 83, wherein antisense oligonucleotide comprises a modification.

89. The antisense oligonucleotide of claim 88, wherein the modification comprises a modified inter-nucleoside linkage, a modified nucleoside, or a combination thereof.

90. The antisense oligonucleotide of claim 89, wherein the modified inter-nucleoside linkage is a phosphorothioate inter-nucleoside linkage and/or a phosphodiester inter-nucleoside linkage.

91. The antisense oligonucleotide of claim 89, wherein the modified nucleoside comprises a modified sugar, optionally wherein the modified sugar is a bicyclic sugar.

92. The antisense oligonucleotide of claim 91, wherein the modified sugar comprises a 2′-O-methoxyethyl group.

93. The antisense oligonucleotide of claim 83, wherein the FOXG1 nucleic acid comprises a 5′ untranslated region (5′ UTR) and a 3′ untranslated region (3′ UTR), and wherein the target sequence is located at the 5′ UTR or the 3′ UTR of the FOXG1 nucleic acid.

94. The antisense oligonucleotide of claim 83, wherein the FOXG1 nucleic acid molecule is a ribonucleic acid (RNA).

95. A pharmaceutical composition comprising the antisense oligonucleotide of claim 83 and a pharmaceutically acceptable carrier or diluent.

96. A method of modulating expression of a FOXG1 in a cell, comprising contacting the cell with a composition comprising an antisense oligonucleotide sequence that hybridizes to a target nucleic acid sequence located within positions 2000-2100 or 2900-3000 of a FOXG1 nucleic acid.

97. The method of claim 96, wherein the cell is a located in a brain of an individual.

98. The method of claim 96, wherein the individual is a human.

99. The method of claim 97, wherein the individual comprises a mutated FOXG1 gene.

100. The method of claim 97, wherein the individual has a FOXG1 disease or disorder.

101. The method of claim 100, wherein the FOXG1 disease or disorder is FOXG1 syndrome.

102. A method of treating or ameliorating a FOXG1 disease or disorder in an individual having, or at risk of having, the FOXG1 disease or disorder, comprising administering to the individual an antisense oligonucleotide, wherein the antisense oligonucleotide comprises a sequence that hybridizes to a target nucleic acid sequence located within positions 2000-2100 or 2900-3000 of a FOXG1 nucleic acid, thereby treating or ameliorating a FOXG1 disease in the individual.
